(19) United States
(12) Patent Application Publication
(10) Pub. No.: US 2023/0055130 A1
(43) Pub. Date: Feb. 23, 2023

(54) MYCELIUM MATERIALS, AND METHODS FOR PRODUCTION THEREOF

(71) Applicant: BOLT THREADS, INC., Emeryville, CA (US)

(72) Inventors: Matthew Jordan Smith, Emeryville, CA (US); Joshua Tyler Kittleson, Emeryville, CA (US); Diana J. Eng, Emeryville, CA (US); Maxime Boulet-Audet, Emeryville, CA (US)

(21) Appl. No.: 17/792,176

(22) PCT Filed: Dec. 16, 2020
(86) PCT No.: PCT/IB20/62051

§ 371 Date: Jul. 12, 2022

Related U.S. Application Data
(60) Provisional application No. 62/951,022, filed on Dec. 20, 2019.

(51) Int. Cl. C12N 1/14; C12N 1/02; C12N 11/00

(52) U.S. Cl. CPC C12N 1/145; C12N 1/02; C12N 11/00; C12R 2001/645

(57) ABSTRACT

Provided herein are mycelium materials and methods for production thereof. In some embodiments, a mycelium material includes: a cultivated mycelium material including one or more masses of branching hyphae, wherein the one or more masses of branching hyphae may be disrupted and/or a bonding agent may be combined with the cultivated mycelium material. Methods of producing a mycelium material are also provided.

### FIELD

The present disclosure generally relates to various mycelium materials having a grown mycelium component and methods for production thereof to provide favorable mechanical and aesthetic qualities.

### BACKGROUND

Due to its bioefficiency, strength, and low environmental footprint, mycelium is of increasing interest in the next generation of sustainable materials. To this end, various applications have discussed methods of growing networks of enmeshed mycelium both on its own and as a composite material (e.g. enmeshed with particles, fibers, networks of fibers, solid matrix bonding agent, or nonwoven lamina). However, the mycelium materials currently undergoing development have poor mechanical qualities, including susceptibility to delamination and tearing under stress, and poor aesthetic qualities. What is needed, therefore, are improved mycelium materials with favorable mechanical properties, aesthetic properties, and other advantages, as well as materials and methods for making improved mycelium materials.

### SUMMARY

Provided herein, according to some embodiments, are various mycelium materials and methods for production thereof to provide mycelium materials and composite mycelium materials with favorable mechanical and aesthetic qualities, and related advantages.

In some embodiments, provided herein is a composite mycelium material. In some embodiments, the composite mycelium material, including: a cultivated mycelium material including one or more masses of branching hyphae, wherein the one or more masses of branching hyphae is disrupted; and a bonding agent. In some embodiments, the cultivated mycelium material has been generated on a solid substrate. In some embodiments, the cultivated mycelium material includes one or more masses of disrupted branching hyphae. In some embodiments, the one or more masses of disrupted branching hyphae has a length of 0.1 mm to 5 cm. In some embodiments, the one or more masses of disrupted branching hyphae has a length of 2 mm.

In some embodiments, the mycelium material further includes a supporting material. In some embodiments, the supporting material has a pore size of ⅛th of an inch (about 3.2 mm) to 1/32nd of an inch (about 0.8 mm). In some embodiments, the supporting material has a pore size of 1/16th of an inch (about 1.6 mm). In some embodiments, supporting material includes a reinforcing material. In some embodiments, the reinforcing material is entangled within the mycelium material. In some embodiments, the supporting material includes a base material. In some embodiments, the base material is positioned on one or more surfaces of the mycelium material. In some embodiments, the supporting material is selected from the group consisting of a mesh, a cheesecloth, a fabric, a knit, a woven, and a non-woven textile.

In some embodiments, the one or more masses of branching hyphae is disrupted by a mechanical action. In some embodiments, the mechanical action includes blending the one or more masses of branching hyphae. In other embodiments, the mechanical action includes applying a physical force to the one or more masses of branching hyphae such that at least some of the masses of branching hyphae are aligned in a parallel formation. In some embodiments, the physical force is a pulling force. In some embodiments, the mechanical action includes applying the physical force in one or more directions such that the at least some of the masses of branching hyphae are aligned in parallel in one or more directions, wherein the physical force is applied repeatedly. In some embodiments, the one or more masses of branching hyphae is disrupted by chemical treatment. In some embodiments, the chemical treatment includes contacting the one or more masses of branching hyphae with a base or other chemical agent in an amount sufficient to cause a disruption. In some embodiments, the base includes alkaline peroxide.

In some embodiments, also provided herein is a bonding agent for a composite mycelium material. In some embodiments, the bonding agent includes one or more reactive groups. In some embodiments, the one or more reactive groups react with active hydrogen containing groups. In some embodiments, the active hydrogen containing groups comprise amine, hydroxyl, and carboxyl groups. In some embodiments, the bonding agent includes an adhesive, a binder, a resin, a crosslinking agent, and/or a matrix. In some embodiments, the bonding agent is selected from the group consisting of transglutaminase, polyamide-epichlorohydrin resin (PAE), citric acid, genipin, alginate, a natural adhesive, and a synthetic adhesive. In some embodiments, the bonding agent is PAE. In some embodiments, the PAE includes cationic azetidinium groups that react with active hydrogen containing groups including amine, hydroxyl, and carboxyl groups, in the one or more branches of hyphae. In some embodiments, the natural adhesive includes a natural latex-based adhesive. In some embodiments, the natural latex-based adhesive is leather glue or weld.

In some embodiments, also provided herein is a mycelium material including one or more proteins that are from a species other than a fungal species from which the cultivated mycelium material is generated. In some embodiments, the one or more proteins is from a plant source. In some embodiments, the plant source is a pea plant. In some embodiments, the plant source is a soybean plant.

In some embodiments, also provided herein is a mycelium material including a dye. In some embodiments, the dye is selected from the group including an acid dye, a direct dye, a synthetic dye, a natural dye, a direct dye, and a reactive dye. In some embodiments, the mycelium material is colored with the dye and the color of the mycelium material is substantially uniform on one or more surfaces of the mycelium material. In some embodiments, the dye is present throughout the interior of the mycelium material.

In some embodiments, also provided herein is a mycelium material including a plasticizer. In some embodiments, the plasticizer is selected from the group including oil, glycerin, fat liquor, water, glycol, triethyl citrate, water, acetylated monoglycerides, epoxidized soybean oil, polyol, and the like. In some embodiments, the mycelium material is flexible. In some embodiments, an external force is applied to the cultivated mycelium material. In some embodiments, the external force is applied via heating and/or pressing.

In some embodiments, also provided herein is a mycelium material including a tannin. In some embodiments, also provided herein is a mycelium material including a finishing agent. In some embodiments, the finishing agent is selected from the group consisting of urethane, wax, nitrocellulose, and a plasticizer

In some embodiments, also provided herein is a mycelium material including a mechanical property. In some embodiments, the mechanical property includes a wet tensile strength, an initial modulus, an elongation percentage at the break, a thickness, and/or a slit tear strength. In some embodiments, the mycelium material has a wet tensile strength of 0.05 MPa to 10 MPa. In some embodiments, the mycelium material has a wet tensile strength of 5 MPa to 20 MPa. In some embodiments, the mycelium material has a wet tensile strength of 7 MPa. In some embodiments, the mycelium material has an initial modulus of 1 MPa to 100 MPa. In some embodiments, the mycelium material has an elongation percentage at the break of 1% to 25%. In some embodiments, the mycelium material has a thickness of 0.5 mm to 3.5 mm. In some embodiments, the mycelium material has a thickness of 2 mm. In some embodiments, the mycelium material has a slit tear strength of 5 N to 100 N. In some embodiments, the mycelium material has a slit tear strength of 50 N. In some embodiments, the mycelium material is produced using traditional paper milling equipment.

According to some embodiments, also provided herein is a method of producing the composite mycelium materials described herein, the method including generating a cultivated mycelium material including one or more masses of branching hyphae; disrupting the cultivated mycelium material including the one or more masses of branching hyphae; and adding a bonding agent to the cultivated mycelium material; thus producing the composite mycelium material. In some embodiments, the generating includes generating cultivated mycelium material on a solid substrate. In some embodiments, the method further includes incorporating a supporting material into the composite mycelium material. In some embodiments, the disrupting includes disrupting the one or more masses of branching hyphae by a mechanical action. In some embodiments, the method further includes adding one or more proteins that are from a species other than a fungal species from which the cultivated mycelium material is generated. In some embodiments, the method further includes adding a dye to the cultivated mycelium material or the composite mycelium material. In some embodiments, the method further includes adding a plasticizer to the cultivated mycelium material or the composite mycelium material. In some embodiments, the method further includes adding a tannin to the cultivated mycelium material or the composite mycelium material. In some embodiments, the method further includes adding a finishing agent to the composite mycelium material. In some embodiments, the method further includes determining a mechanical property of the composite mycelium material.

According to some embodiments of the present disclosure, a method of producing a material including mycelium is provided. The method includes introducing a fungal inoculum and a nutrient source to a vessel of a bioreactor, wherein the nutrient source is compatible with the fungal inoculum for consumption by the same. A liquid is introduced to the vessel to provide a mixture and the mixture is incubated in the bioreactor under aerobic conditions to grow a biomass of mycelium having a plurality of branches of hyphae having a length of at least about 0.1 mm. After the step of incubating the mixture, at least a portion of the biomass of mycelium is collected and a concentration of the biomass of mycelium is adjusted to a predetermined concentration. The method also includes web-forming the collected biomass of mycelium to form a hyphal network.

According to some embodiments of the present disclosure, a method of producing a material including mycelium is provided. The method includes introducing a fungal inoculum and a nutrient source to a vessel of a bioreactor, wherein the nutrient source is compatible with the fungal inoculum for consumption by the same. A liquid is introduced to the vessel to provide a mixture, wherein the liquid comprises a surfactant that is a polymeric macromolecule including monomer units selected from at least one of propylene oxide and ethylene oxide. The mixture is incubated in the bioreactor under aerobic conditions to grow a biomass of mycelium having a plurality of branches of hyphae having a length of at least about 0.1 mm. After the step of incubating the mixture, at least a portion of the biomass of mycelium is collected and a concentration of the biomass of mycelium is adjusted to a predetermined concentration. The method also includes drying the biomass of mycelium.

### BRIEF DESCRIPTION OF THE FIGURES

FIG. 1 depicts a schematic diagram of methods of producing a mycelium material, according to aspects of the present disclosure.

FIG. 2 depicts stress-strain curves of a pressed sample with polyamide-epichlorohydrin resin (PAE) and scaffold3 (dashed lines), and a pressed sample with PAE and scaffold4 (solid lines), according to aspects of the present disclosure. Standard force (MPa) is plotted against elongation at the break (%).

FIG. 3 depicts different scaffold materials, according to aspects of the present disclosure. From left to right: scaffold1, a cheesecloth scaffold with pores slightly smaller than 1/16th of an inch; scaffold2, a cotton textile scaffold with pores smaller than 1/32nd of an inch; scaffold3, a non-textile scaffold with pores 1/16th of an inch in size; and scaffold4 a cotton textile scaffold with large pores ⅛th of an inch in size.

FIG. 4 depicts a sample containing 5 g cultivated mycelium material (indicated by an arrow), 125 mL of 1.5% PAE in 25 mM phosphate buffer (pH=7.4), 1 g pea protein, scaffold4, and Weldwood® adhesive after a wet tensile test, according to aspects of the present disclosure.

FIG. 5 depicts a plot of slit tear (N) versus thickness (mm) of pressed samples (HM1-4-3 and HM1-1-11_120 p) and unpressed samples (HM1-1-1, HM1-1-7, and HM1-1-11), according to aspects of the present disclosure.

FIG. 6 depicts a stress-strain curve plotting engineering stress (MPa) against nominal strain (%), according to aspects of the present disclosure. The strain cycles from 10% to 80% in increments of 10% before drafting to the maximum elongation.

FIGS. 7A and 7B depict SEM micrographs of mycelium hyphae before drafting (FIG. 7A) and after drafting (FIG. 7B), according to aspects of the present disclosure. Scale bar of FIG. 7A=50 μm; scale bar of FIG. 7B=200 μm.

FIG. 8 shows a Fourier transform graph of a mycelium SEM image before drafting (black squares) and after drafting (grey circles), according to aspects of the present disclosure.

FIG. 9 shows a polarized Fourier transform infrared spectroscopy (FTIR) spectra graph of normalized absorbance versus wavenumber 1/cm of aligned mycelium hypha along with the polarization (0 degrees) and perpendicular to the polarization (90 degrees), according to aspects of the present disclosure. A spectrum of pure chitin is shown for comparison.

FIG. 10 depicts a polarized FTIR spectra graph of normalized absorbance versus wavenumber 1/cm of a second Legendre order parameter (<P2>) as a function of the wavenumber for non-aligned and aligned mycelium hypha, according to aspects of the present disclosure.

FIGS. 11A and 11B depicts scanning electron microscope (SEM) micrographs of two aligned mycelium lamina bonded with polyurethane hot melt adhesive at 150× (FIG. 11A) and 500× magnification (FIG. 11B), according to aspects of the present disclosure.

FIGS. 12A and 12B depicts stress-strain curves of aligned mycelium bonded with polyurethane hot melt adhesive tested after conditioning at 65% relative humidity (RH) in a dry state (FIG. 12A) and a wet state (FIG. 12B), according to aspects of the present disclosure.

FIG. 13 depicts a flowchart of a method of producing a material comprising mycelium, according to aspects of the present disclosure.

FIG. 14 depicts a flowchart of a method for converting raw mycelium material into a crust material for a finishing process, according to aspects of the present disclosure.

FIG. 15 depicts photographs of Phycomyces blakesleeanus biomasses grown in a liquid process, according to aspects of the present disclosure.

FIG. 16 depicts photographs of Phycomyces blakesleeanus hyphae grown in a liquid process, according to aspects of the present disclosure.

FIG. 17 depicts photographs of Neurospora crassa hyphae grown in a liquid process, according to aspects of the present disclosure.

FIG. 18 depicts a wet tensile stress-strain curve of Phycomyces blakesleeanus grown in liquid culture, blended, then wet laid and hydroentangled according to aspects of the present disclosure.

FIG. 19 depicts a plot of an agitation profile showing an agitation rate as a function of time during an incubation period of a liquid growth process for producing mycelium material in a reaction vessel from a fungal inoculum, according to aspects of the present disclosure.

FIG. 20 depicts a plot of dissolved oxygen as a function of time during an incubation period of a liquid growth process for producing mycelium material in a reaction vessel from a fungal inoculum, according to aspects of the present disclosure.

FIG. 21 depicts a plot of total glucose consumed per initial volume as a function of time during an incubation period of a liquid growth process for producing mycelium material in a reaction vessel from a fungal inoculum, according to aspects of the present disclosure.

FIG. 22 depicts a plot of the residual glucose concentration as a function of time during an incubation period of a liquid growth process for producing mycelium material in a reaction vessel from a fungal inoculum, according to aspects of the present disclosure.

FIG. 23 depicts a plot of oxygen uptake rate (OUR) as a function of time during an incubation period of a liquid growth process for producing mycelium material in a reaction vessel from a fungal inoculum, according to aspects of the present disclosure.

FIG. 24 depicts a plot of respiratory quotient as a function of time during an incubation period of a liquid growth process for producing mycelium material in a reaction vessel from a fungal inoculum, according to aspects of the present disclosure.

FIG. 25 depicts a plot of ethanol concentration as a function of time during an incubation period of a liquid growth process for producing mycelium material in a reaction vessel from a fungal inoculum, according to aspects of the present disclosure.

FIG. 26 depicts a plot showing an amount of mycelium material produced after incubation periods of about 23 hours, about 27 hours, and about 46 in a liquid growth process, according to aspects of the present disclosure.

FIG. 27 depicts images of RMs2374 N. crassa after germinating for 6 hours in a seed flask (A) and after incubating in a reaction vessel for 24 hours (B), 30 hours (C), and 45 hours (D) in a liquid growth process, according to aspects of the present disclosure.

### DETAILED DESCRIPTION

### Definitions

The details of various embodiments of the disclosure are set forth in the description below. Other features, objects, and advantages of the disclosure will be apparent from the description. Unless otherwise defined herein, scientific and technical terms used in connection with the present disclosure shall have the meanings that are commonly understood by those of ordinary skill in the art. Further, unless otherwise required by context, singular terms shall include the plural and plural terms shall include the singular. The terms “a” and “an” includes plural references unless the context dictates otherwise. Generally, nomenclatures used in connection with, and techniques of, biochemistry, enzymology, molecular and cellular biology, microbiology, genetics and protein and nucleic acid chemistry and hybridization described herein are those well-known and commonly used in the art.

The following terms, unless otherwise indicated, shall be understood to have the following meanings:

The term “hyphae” refers to a morphological structure of a fungus that is characterized by a branching filamentous shape.

The term “hyphal” refers to an object having a component thereof comprised of hyphae.

The term “mycelium” refers to a structure formed by one or more masses of branching hyphae. A “mass” refers to a quantity of matter. Mycelium is a distinct and separate structure from a fruiting body of a fungus or sporocarp.

The terms “cultivate” and “cultivated” refer to the use of defined techniques to deliberately grow a fungus or other organism.

The term “cultivated mycelium material” refers to material that includes one or more masses of cultivated mycelium, or includes solely of cultivated mycelium. In some embodiments, the one or more masses of cultivated mycelium is disrupted as described herein. In most cases, the cultivated mycelium material has been generated on a solid substrate, as described below.

The term “composite mycelium material” refers to any material including cultivated mycelium material combined with another material, such as a bonding agent or a supporting material as described herein, such as a crosslinking agent, natural adhesive, or a synthetic adhesive. In some embodiments, a supporting material is entangled within a mycelium or composite mycelium material, e.g., a reinforcing material. In some embodiments, a supporting material is positioned on one or more surfaces of the mycelium or composite mycelium material, e.g., a base material. Suitable supporting materials include, but are not limited to, a mass of contiguous, disordered fibers (e.g. non-woven fibers), a perforated material (e.g. metal mesh, perforated plastic), a mass of discontiguous particles (e.g. pieces of woodchip) or any combination thereof. In specific embodiments, the supporting material is selected from the group consisting of a mesh, a cheesecloth, a fabric, a knit, a woven, and a non-woven textile.

The term “incorporate” refers to any substance, e.g., cultivated mycelium material, composite mycelium material, or a bonding agent, that can be combined with or contacted with another substance. In a specific embodiment, a mycelium or composite mycelium material can be combined with, contacted with, or incorporated into a supporting material, e.g., woven, twisted, wound, folded, entwined, entangled, or braided together, to produce a mycelium material that has become incorporated with the supporting material. In another embodiment, one or more bonding agents may be incorporated within the cultivated mycelium material to be bonded, either in its disrupted or undisrupted state, e.g., embedded throughout the material, or added as a thin coating layer, such as by spraying, saturation, dipping, nip rolling, coating, and the like, to produce a mycelium material.

As used herein, the term “disrupted” with respect to one or more masses of branching hyphae refer to one or more masses of branching hyphae of which one or more disruptions have been applied. A “disruption,” as described herein, may be mechanical or chemical, or a combination thereof. In some embodiments, the one or more masses of branching hyphae is disrupted by a mechanical action. A “mechanical action” as used herein refers to a manipulation of or relating to machinery or tools. Exemplary mechanical actions include, but are not limited to, blending, chopping, impacting, compacting, bounding, shredding, grinding, compressing, high-pressure, shearing, laser cutting, hammer milling, and waterjet forces. In some embodiments, a mechanical action may include applying a physical force, e.g., in one or more directions such that the at least some of the masses of branching hyphae are aligned in parallel in one or more directions, wherein the physical force is applied repeatedly. In some other embodiments, the one or more masses of branching hyphae is disrupted by chemical treatment. “Chemical treatment” as used herein refers to contacting the cultivated mycelium material or composite mycelium material with a chemical agent, e.g., a base or other chemical agent, in an amount sufficient to cause a disruption. In various embodiments, a combination of mechanical actions and chemical treatments may be used herein. The amount of mechanical action (for example, the amount of pressure) and/or chemical agent applied, the period of time for which the mechanical action and/or chemical treatment is applied, and the temperature at which the mechanical action and/or chemical agent is applied, depends, in part, on the components of the cultivated mycelium material or composite mycelium material, and are selected to provide an optimal disruption on the cultivated mycelium material or composite mycelium material.

The term “plasticizer” as used herein refers to any molecule that interacts with a structure to increase mobility of the structure.

The term “processed mycelium material” as used herein refers to a mycelium that has been post-processed by any combination of treatments with preserving agents, plasticizers, finishing agents, dyes, and/or protein treatments.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range, is encompassed within the aspects of the present disclosure. The upper and lower limits of these smaller ranges may independently be included in the smaller ranges, and are also encompassed within the aspects of the present disclosure, subject to any specifically excluded limit in the stated range. Where the stated range includes one or both of the limits, ranges excluding either or both of those included limits are also included in the aspects of the present disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Exemplary methods and materials are described below, although methods and materials similar or equivalent to those described herein can also be used in the practice of the present disclosure and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Mycelium Compositions and Methods of Production

Provided herein are cultivated mycelium materials and composite mycelium materials and scalable methods of producing the cultivated mycelium materials and composite mycelium materials. In some or most embodiments, the composite mycelium materials include a cultivated mycelium material having one or more masses of branching hyphae, wherein the one or more masses of branching hyphae is disrupted, and a bonding agent. Methods of producing the cultivated mycelium material and composite mycelium material are also provided.

Exemplary patents and applications discussing methods of growing mycelium include, but are not limited to: WIPO Patent Publication No. 1999/024555; G.B. Patent No. 2,148,959; G.B. Patent No. 2,165,865; U.S. Pat. Nos. 5,854,056; 2,850,841; 3,616,246; 9,485,917; 9,879,219; 9,469,838; 9,914,906; 9,555,395; U.S. Patent Publication No. 2015/0101509; U.S. Patent Publication No. 2015/0033620, all of which are incorporated herein by reference in their entirety. U.S. Patent Publication No. 2018/0282529, published on Oct. 4, 2018 discusses various mechanisms of solution-based post-processing mycelium material to produce a material that has favorable mechanical characteristics for processing into a textile or leather alternative.

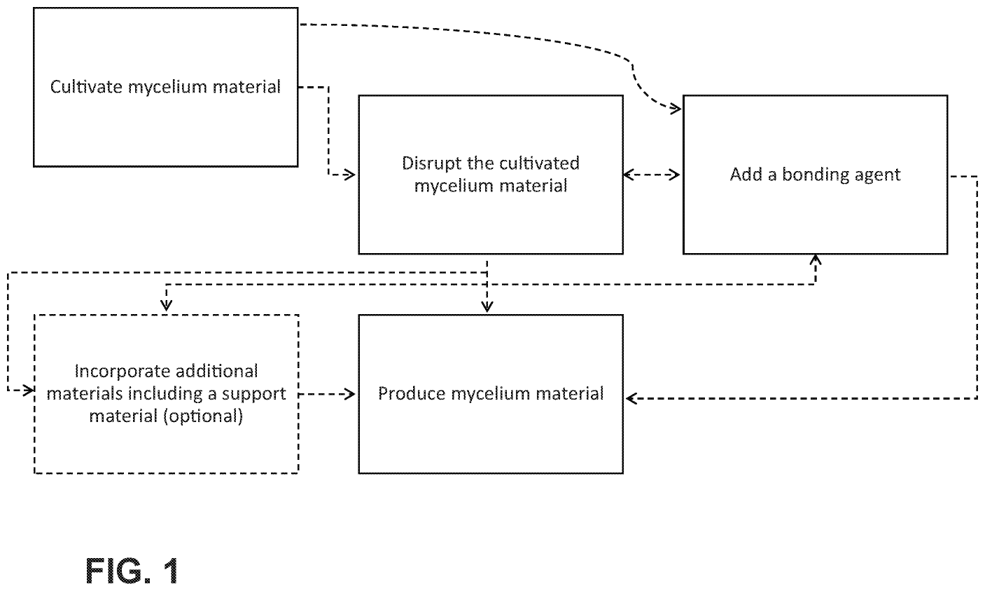

As shown in FIG. 1, exemplary methods of producing mycelium materials according to some embodiments described herein include cultivating mycelium material, optionally disrupting cultivated mycelium material, optionally adding a bonding agent, optionally incorporating additional materials such as a support material, and combinations thereof. In various embodiments, traditional paper milling equipment may be adapted or used to perform some, or all, of the steps presented herein. In such embodiments, the mycelium material is produced using traditional paper milling equipment.

A description of an embodiment with several components in communication with each other does not imply that all such components are required. To the contrary, a variety of optional components may be described to illustrate a wide variety of possible embodiments of one or more aspects of the present disclosure and in order to more fully illustrate one or more aspects of the present disclosure. Similarly, although process steps, method steps, algorithms or the like may be described in sequential order, such processes, methods, and algorithms may generally be configured to work in alternate orders, unless specifically stated to the contrary. In other words, any sequence or order of steps that may be described herein does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more embodiments, and does not imply that the illustrated process is preferred. Also, steps are generally described once per embodiment, but this does not mean they must occur once, or that they may only occur once each time a process, method, or algorithm is carried out or executed. Some steps may be omitted in some embodiments or some occurrences, or some steps may be executed more than once in a given embodiment or occurrence.

### Cultivated Mycelium Material

Embodiments of the present disclosure include various types of cultivated mycelium materials. Depending on the particular embodiment and requirements of the material sought, various known methods of cultivating mycelium may be used. Any fungus that can be cultivated as mycelium may be used. Suitable fungus species for use include, but are not limited to: Agaricus arvensis; Agrocybe brasiliensis; Amylomyces rouxii; Amylomyces sp.; Armillaria mellea; Aspergillus nidulans; Aspergillus niger; Aspergillus oryzae; Ceriporia lacerata; Coprinus comatus; Fibroporia vaillantii; Fistulina hepatica; Flammulina velutipes; Fomitopsis officinalis; Ganoderma sessile; Ganoderma tsugae; Hericium erinaceus; Hypholoma capnoides; Hypholoma sublateritium; Inonotus obliquus; Lactarius chrysorrheus; Macrolepiota procera; Morchella angusticeps; Myceliophthora thermophila; Neurospora crassa; Penicillium camemberti; Penicillium chrysogenum; Penicillium rubens; Phycomyces blakesleeanus; Pleurotus djamor; Pleurotus ostreatus; Polyporus squamosus; Psathyrella aquatica; Rhizopus microsporus; Rhizopus oryzae; Schizophyllum commune; Streptomyces venezuelae; Stropharia rugosoannulata; Thielavia terrestris; Ustilago maydis; Fusarium sp.; and Gibberella sp. In some embodiments, the fungus used includes Ganoderma sessile, Neurospora crassa, and/or Phycomyces blakesleeanus. In some embodiments, the fungus is a mutant strain of any of the above-identified fungal species. In an exemplary embodiment, the fungus is RMs2374, a Neurospora crassa mutant that can be obtained from the Fungal Genetics Stock Center (FGSC 5140).

In some embodiments, the strain or species of fungus may be bred to produce cultivated mycelium material with specific characteristics, such as a dense network of hyphae, a highly-branched network of hyphae, hyphal fusion within the network of hyphae, and other characteristics that may alter the properties of the cultivated mycelium material. In some embodiments, the strain or species of fungus may be genetically modified to produce cultivated mycelium material with specific characteristics.

In most embodiments, the cultivated mycelium may be grown by first inoculating a solid or liquid substrate with an inoculum of the mycelium from the selected species of fungus. In some embodiments, the substrate is pasteurized or sterilized prior to inoculation to prevent contamination or competition from other organisms. For example, a standard method of cultivating mycelium includes inoculating a sterilized solid substrate (e.g. grain) with an inoculum of mycelium. Other standard methods of cultivating mycelium include inoculating a sterilized liquid medium (e.g. liquid potato dextrose) with an inoculum of mycelium or a pure cultured spawn. In some embodiments, the solid and/or liquid substrate will include lignocellulose as a carbon source for mycelium. In some embodiments, the solid and/or liquid substrate will contain simple or complex sugars as a carbon source for the mycelium.

According to an embodiment of the present disclosure, cultivated mycelium may be grown in a liquid process in a bioreactor. The bioreactor may be bench scale or industrial scale, with the bench scale parameters optionally providing the basis for scaling up the liquid growth process. The parameters of the liquid growth process are selected to provide a mycelial biomass having morphological characteristics suitable for producing mycelium materials having the desired mechanical and/or aesthetic properties described herein. According to one aspect of the present disclosure, the liquid growth process described herein can be used to form hyphal networks having suitable morphology for entanglement of the hyphae, and more specifically, suitable for entanglement through a mechanical entanglement process, such as a hydroentanglement process. The mycelial biomass produced according to the liquid growth processes described herein can be further processed according to any of the downstream post-processing methods described herein.

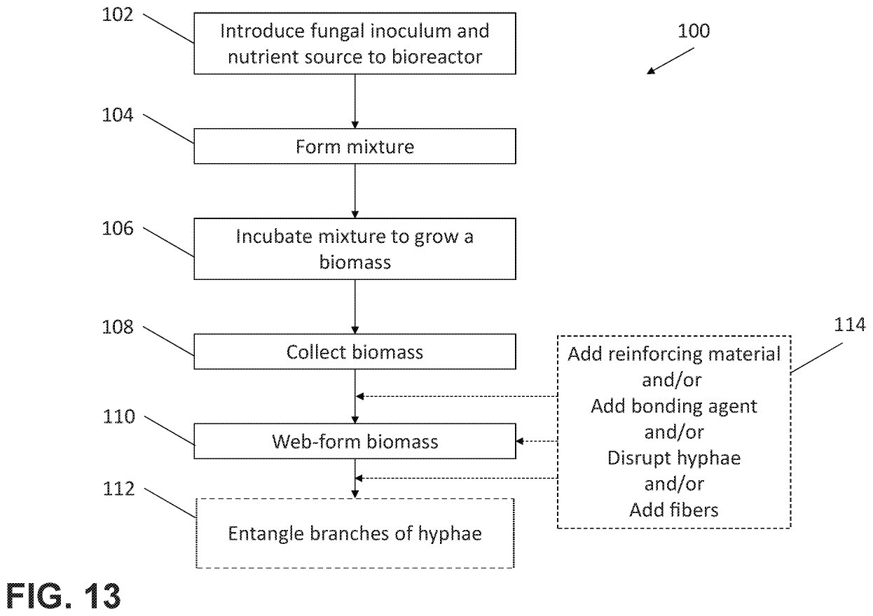

Referring now to FIG. 13, a method 100 for producing a mycelium material is illustrated. The method 100 can be implemented in a bioreactor having a reaction vessel of a desired size (e.g., bench top scale or large scale). The method 100 includes introducing a fungal inoculum and nutrient source to a bioreactor at 102, forming a mixture within a vessel of the bioreactor at 104, incubating the mixture to grow a biomass of mycelium at 106, collecting the cultivated biomass of mycelium at 108, web-forming the biomass of mycelium at 110 to form a hyphal network. The method 100 can optionally include entangling branches of hyphae in the hyphal network at 112 and/or optional step(s) 114 including (a) adding a reinforcing material, (b) adding a bonding agent, (c) disruption of the hyphae, and/or (d) addition of fibers, as discussed herein.

Still referring to FIG. 13, the vessel may be made from any suitable coated or uncoated non-porous material, such as glass or stainless steel, for example. The vessel may optionally be insulated, water-jacketed for heating and/or cooling, and/or include a heating element for heating the contents of the vessel. The method 100 includes introducing a fungal inoculum and a nutrient source (also referred to as a nutritional source) to a vessel of a bioreactor at step 102. The fungal inoculum can be any inoculum of the desired fungal species, examples of which are provided above. In one exemplary embodiment, the fungal species is a mutant of Neurospora crassa, such as Neurospora crassa RMs2374, for example. RMs2374 is a classical mutant of N. crassa that exhibits an albino phenotype and can be obtained from the Fungal Genetics Stock Center (FGSC 5140).

The nutrient source is selected to be compatible with the selected fungal inoculum such that the nutrient source can be consumed by the fungal inoculum to support growth of a biomass of mycelium having a plurality of branches of hyphae. Examples of suitable nutrient sources are disclosed below. At step 104, a liquid can be introduced into the reaction vessel to form a mixture having the desired volume and/or concentration of inoculum and nutrients. Steps 102 and 104 may occur sequentially or simultaneously.

According to an aspect of the present disclosure, the fungal inoculum can be a spore solution or live mycelium that is added to the vessel of the bioreactor. In some aspects, the spore solution that is added to the bioreactor vessel is contains fresh spores. As used herein, the term “fresh,” when used to describe spores in a spore solution or fungal inoculum refers to a solution containing spores that have been cultured, harvested, and used to inoculate a vessel in a liquid growth process without being stored at a temperature of 4° C. or less between the process of harvesting the spores and the process of inoculating the vessel with the spores. In other words, the cultured spores are used to inoculate a vessel without decreasing a temperature of the spores to 4° C. or less (e.g., by freezing or refrigerating the spores) between culturing the spores and inoculating a vessel with the spores to grow a mycelium biomass according to aspects of the present disclosure in a liquid growth process. Without wishing to be limited by any theory, it has been found that introducing a fresh fungal inoculum that has not been frozen or refrigerated into the vessel (i.e., that has not been placed in low temperature storage for a time period sufficient to decrease a temperature of the spores and/or the material in which the spores are suspended in to 4° C. or less) can facilitate the growth of a mycelium biomass having the desired morphological features (e.g., hyphal length). In some aspects, the use of a fresh fungal inoculum that has not been frozen or refrigerated facilitates producing a desired yield of mycelium biomass within a desired period of time. It has been found that for some fungal species, for example N. crassa RMs2374, storing the spore solution or live mycelium at a low temperature prior to inoculation can result in a mycelium biomass having undesired features (e.g., clumping, low dispersion) and/or a slower growth rate compared to inoculation with a fresh spore solution or live mycelium that has not been stored at low temperatures. The slower growth rate and/or lower yield of a suitable mycelium biomass can become time and/or cost prohibitive as the reaction scale increases to larger vessels, such as those that are suitable for large-scale manufacturing (e.g., 50 L to 300 L vessels).

An exemplary process for preparing a fresh spore solution for inoculating the bioreactor vessel can include seeding flasks containing a suitable media and growth material (e.g., agar) based on the fungal species. The incubation time period and temperature for culturing the fungal spores to produce a desired number of spores for the inoculation step 102 can vary depending on the fungal species. For example, N. crassa can be seeded and incubated at about 30° C. for 3 days, after which the seed flasks are transferred to room temperature (about 25° C.) and left stationary for about a week to allow conidia to form. Once a suitable amount of visible clumps or chains of conidia appear, the conidia can be harvested from the seed flasks and optionally strained through a sterile mesh to remove large chunks of mycelia (e.g., 40 μm sterile mesh). The collected spore solution can be concentrated, diluted, and/or combined as needed to provide a spore solution that forms a fungal inoculum having a desired concentration for the vessel inoculation at step 102. As discussed above, in some aspects, the spore solution can be stored at room temperature (or warmer) prior to inoculating the vessel at step 102. In other words, the spore solution is a fresh spore solution that is not frozen or refrigerated (i.e., stored at 4° C. or less) prior to inoculating the vessel. For example, the spores can be harvested the same day as inoculation occurs and stored at room temperature (or warmer) in the time period between harvesting the spores to generate the spore solution and inoculating the reaction vessel. The desired spore concentration can vary based on a variety of factors, such as the fungal species, growth rate, and/or vessel size. In some aspects, the target concentration of spores in the inoculating solution is at least about 10⁵ spores/mL, at least about 10⁶ spores/mL, at least about 10⁷ spores/mL, or at least about 10⁸ spores/mL. In one aspect, the target concentration of spores in the inoculating solution is from about 10⁵ spores/mL to about 10⁸ spores/mL or about 10⁶ spores/mL to about 10⁸ spores/mL.

The mixture formed in the reaction vessel at step 104 may include components in addition to the inoculum and the nutrient source. For example, the mixture can optionally include a carbon source, a nitrogen source, a pH modifier, trace minerals, surfactants and/or a nutrient supplement. Examples of a carbon source include glucose, xylose, and lactose. Examples of a nitrogen source include ammonium nitrate, ammonium sulfate, ammonium chloride, and amino acids. Examples of surfactants include polyethylene glycols and polysorbate 80. The pH modifier can be any suitable buffer that facilitates maintaining the mixture at the desired pH or within a desired pH range. The pH or pH range of the mixture can be selected to promote growth based on the specific fungal species. In some examples, the pH of the mixture is from about 3 to about 8, about 4 to about 8, about 5 to about 8, about 6 to about 8, about 3 to about 4, about 3 to about 5, about 3 to about 6, about 3 to about 7, about 4 to about 5, about 4 to about 6, about 4 to about 7, about 6 to about 7, about 5 to about 6, about 5 to about 7, or about 7 to about 8. The pH modifier can be selected to facilitate maintaining the desired pH or pH range during the subsequent incubation step 106. Trace minerals can include iron (e.g., ammonium iron (II) sulfate hexahydrate), zinc (e.g., zinc sulfate heptahydrate), copper (e.g., copper sulfate pentahydrate), manganese (e.g., manganese sulfate monohydrate), molybdenum (e.g., sodium molybdate dihydrate), and combinations thereof. The nutrient supplement can be selected based on the specific fungal species and can include one or more vitamins, examples of which include biotin and thiamine.

According to one aspect, a surfactant is added to the reaction vessel with the spores. The surfactant can be a component of the media within which the spores are suspended or a separate component added to the reaction vessel. In some aspects, the surfactant can be a polymeric macromolecule including at least ethylene oxide and/or propylene oxide monomer units, non-limiting examples of which include propylene oxide polymers, propylene oxide block co-polymers, propylene oxide/ethylene oxide block co-polymers, polyether polyols, polypropylene glycol, and combinations thereof. In some aspects, the surfactant may be a nonionic surfactant, such as a nonionic polyol or nonionic homopolymer diol. In one example, the surfactant can be Dow's TERGITOL™ L-81, which is described as a polyether polyol having ethylene oxide/propylene oxide block co-polymer chemistry. In another example, the surfactant can be Dow's Polyglycol P 2000, which is described as a propylene glycol initiated homopolymer diol having a molecular weight of 2000 g/mol. Without wishing to be limited by any theory, it has been found that for some fungal species, such as N. crassa and mutants thereof, certain surfactants facilitate the growth of a biomass having the desired characteristics, such as a desired morphology and/or dispersion. The polymeric macromolecule surfactants of the present disclosure including at least ethylene oxide and/or propylene oxide monomer units have been found to inhibit the formation of clumps associated with components of the reaction vessel (e.g., agitator, shafts, vessel walls, etc.), which are believed to inhibit growth of a biomass having the desired morphology. For example, it was observed that some types of surfactants, for example Tween 80 (also referred to as Polysorbate 80), did not inhibit the formation of foam and/or clumps in the reaction vessel (based on visual observation) and in some cases was observed to result in an increase in foaming compared to a media that was free of surfactants. Clumping and/or foaming may be amplified as the size of the vessel reactor increases and thus could restrict the ability to scale up the vessel reactor size, as would likely be required in a large-scale manufacturing setting. Fungal species and vessel conditions that do not produce a minimum amount of a mycelium biomass having the desired morphological characteristics (e.g., hyphal length, dispersion, etc.) within a suitable time period may not be capable of meeting the production time and costs requirements typical for large-scale production. The addition of a suitable surfactant according to the present disclosure may inhibit or minimize the formation of clumps of mycelia, which may facilitate large-scale production of sufficient amounts of mycelium biomass material having the desired morphological characteristics.

In some aspects, the polymeric macromolecule surfactant including at least ethylene oxide and/or propylene oxide monomer units of the present disclosure may be present in the mixture in an amount of from about 0.2 mL/L to about 5 mL/L. For example, the polymeric macromolecule surfactant may be present in the mixture in an amount of from about 0.2 mL/L to about 5 mL/L, about 0.2 mL/L to about 4 mL/L, about 0.2 mL/L to about 3 mL/L, about 0.2 mL/L to about 2 mL/L, about 0.2 mL/L to about 1 mL/L, about 0.2 mL/L to about 0.5 mL/L, about 0.5 mL/L to about 5 mL/L, about 0.5 mL/L to about 4 mL/L, about 0.5 mL/L to about 3 mL/L, about 0.5 mL/L to about 2 mL/L, about 0.5 mL/L to about 1 mL/L, about 1 mL/L to about 5 mL/L, about 1 mL/L to about 4 mL/L, about 1 mL/L to about 3 mL/L, about 1 mL/L to about 2 mL/L, about 2 mL/L to about 5 mL/L, about 2 mL/L to about 4 mL/L, or about 2 mL/L to about 3 mL/L. In some aspects, the polymeric macromolecule surfactant of the present disclosure may be present in the mixture in the vessel in an amount of from about 0.01% to about 1% by weight (wt %). For example, the polymeric macromolecule surfactant may be present in the mixture in an amount of from about 0.01 wt % to about 1 wt %, about 0.01 wt % to about 0.75 wt %, about 0.01 wt % to about 0.5 wt %, about 0.01 wt % to about 0.4 wt %, about 0.01 wt % to about 0.25 wt %, about 0.01 wt % to about 0.1 wt %, about 0.025 wt % to about 1 wt %, about 0.025 wt % to about 0.75 wt %, about 0.025 wt % to about 0.5 wt %, about 0.025 wt % to about 0.4 wt %, about 0.025 wt % to about 0.25, about 0.025 wt % to about 0.1 wt %, about 0.05 wt % to about 1 wt %, about 0.05 wt % to about 0.75 wt %, about 0.05 wt % to about 0.5 wt %, about 0.05 wt % to about 0.4 wt %, about 0.05 wt % to about 0.25, about 0.05 wt % to about 0.1 wt %, about 0.1 wt % to about 1 wt %, about 0.1 wt % to about 0.75 wt %, about 0.1 wt % to about 0.5 wt %, about 0.1 wt % to about 0.4 wt %, about 0.1 wt % to about 0.25, about 0.25 wt % to about 1 wt %, about 0.25 wt % to about 0.75 wt %, about 0.25 wt % to about 0.5 wt %, about 0.25 wt % to about 0.4 wt %, about 0.4 wt % to about 1 wt %, about 0.4 wt % to about 0.75 wt %, or about 0.4 wt % to about 0.5 wt %. In some examples, the polymeric macromolecule surfactant is Dow's Polyglycol P 2000 and is present in the mixture in an amount of from about 0.05 wt % to about 0.4 wt %.

According to one aspect of the present disclosure, a fed-batch fermentation method is used. The mixture may be constantly supplied with one or more nutrients while maintaining conditions such as temperature, oxygen levels, and pH levels. According to another aspect of the present disclosure, a set of nutrient limitations may be used. In one embodiment, the amount of nitrogen is limited. In one other embodiment, the amount of oxygen is limited and the oxygen transfer rate (OTR) is influenced. For example, one or more conditions of the contents of the vessel can be monitored and used to adjust one or more conditions of the fermentation process and/or used to determine an amount of one or more materials to add to the vessel. In some embodiments, the mixture may be provided with a supply of glucose or other nutrient constantly or intermittently according to a predetermined schedule or based on a measured condition of the reaction vessel.

According to one aspect, the mixture in the reaction vessel can include a nutrient source containing an initial amount of at least one nutrient, such as glucose, for example, that is consumed as the biomass grows. Once a predetermined portion of the initial amount of the nutrient(s) has been consumed, additional nutrient(s) can be fed into the reaction vessel intermittently and/or at a constant rate to facilitate continued growth of the biomass. The predetermined portion of the initial amount of nutrient(s) used to determine the supply of additional nutrients may correspond to an entirety of the initial amount of the nutrient (i.e., 100% of the initial amount of nutrient) or some amount less than an entirety of the initial amount of the nutrient. For example, the supply of additional nutrient(s) may be based on consumption of at least about 50%, at least about 60%, at least about 70%, at least about 80%, at least about 90%, at least about 95%, at least about 99%, or at least about 100% of the initial amount of the nutrient(s). The initial amount of the nutrient(s), amount of additional nutrient(s) added, schedule of addition, and/or rate of supply of additional nutrient(s) may be based at least in part on the species of fungus. In some aspects, the additional nutrient(s) may be added based at least in part on one or more measured conditions within the reaction vessel, examples of which include nutrient(s) concentration, dissolved oxygen levels, and carbon evolution rate (CER). For example, a spike in dissolved oxygen levels may be correlated with consumption of the initial amount of a nutrient. In another example, a drop in carbon evolution rate CER may be correlated with consumption of the initial amount of a nutrient. In one example, consumption of the initial amount of nutrient(s) may be determined based on an observed spike in dissolved oxygen and a drop in carbon evolution rate (CER), and the supply of additional nutrient(s) may be initiated. It is understood that the one or more measured conditions used to determine the consumption of the initial amount of nutrient(s) may be based on those condition(s) that are indicative of the amount of nutrient(s) consumed and/or the amount of nutrient(s) remaining and that those condition(s) may vary depending on the particular nutrient(s) of interest.

For example, the fungal spores can be added to the reaction vessel in a solution that includes an initial amount of glucose. After this initial amount of glucose has been consumed, additional glucose can be fed into the reaction vessel intermittently or at a constant rate (e.g., 1.8 grams of glucose/liter/hour). The initial amount of glucose, amount of additional glucose added, schedule of addition of glucose, and/or rate of supply of additional glucose may be based at least in part on the species of fungus. In some aspects, the additional glucose may be added based at least in part on one or more measured conditions within the reaction vessel, examples of which include glucose concentration, dissolved oxygen levels, and carbon evolution rate. In one example, consumption of the initial glucose may be determined based on an observed increase in dissolved oxygen and/or a decrease in carbon evolution rate (CER), and the supply of additional glucose may be initiated.

At step 106, the thus formed mixture within the bioreactor vessel is incubated to promote growth of the mycelium biomass. The conditions of the bioreactor can be selected to promote growth of a mycelium biomass having a plurality of branches of hyphae having sufficient morphological characteristics for entanglement in a downstream process. Example morphological characteristics include a minimum length of hyphae branches, a desired density of the hyphae network, a desired degree of branching of the hyphae, a desired aspect ratio, and/or a desired degree of hyphal fusion of the hyphae network. According to one aspect of the present disclosure, the conditions of the bioreactor in the incubating step at 106 are selected to promote growth of a biomass of mycelium having a plurality of branches of hyphae having a length of at least about 0.1 mm. For example, the hyphae can have a length of from about 0.1 mm to about 5 mm, about 0.1 mm to about 4 mm, about 0.1 mm to about 3 mm, about 0.1 mm to about 2 mm, about 0.1 mm to about 1 mm, about 0.2 mm to about 5 mm, about 0.2 mm to about 4 mm, about 0.2 mm to about 3 mm, about 0.2 mm to about 2 mm, about 0.2 mm to about 1 mm, about 1 mm to about 5 mm, about 1 mm to about 4 mm, about 1 mm to about 3 mm, about 1 mm to about 2 mm, about 2 mm to about 5 mm, about 2 mm to about 4 mm, or about 2 mm to about 3 mm. In some aspects, the hyphae can have a length of at least about 0.1 mm, at least about 0.125 mm, at least about 0.15 mm, at least about 0.175, at least about 0.2 mm, at least about 0.2, at least about 0.225 mm, at least about 0.25 mm, at least about 0.275 mm, or at least about 0.3 mm.

The incubation step 106 can occur under aerobic conditions in the presence of oxygen. Optionally, the reaction vessel can be sealed during all or a portion of the incubation step. In some examples, oxygen may be introduced into the reaction vessel. The incubation temperature can be selected based on the specific fungal species. In some examples, the temperature of the mixture during incubation is from about 20° C. to about 40° C., about 25° C. to about 40° C., about 30° C. to about 40° C., about 35° C. to about 40° C., about 20° C. to about 35° C., about 25° C. to about 35° C., about 30° C. to about 35° C., about 20° C. to about 30° C., or about 25° C. to about 30° C.

The incubation step 106 may include agitation of the mixture during at least a portion of the incubation period. In some examples, agitation occurs during the entire incubation period. In other examples, the incubation period may not include any agitation. Examples of agitation include rotation of an impeller, movement of an agitator, shaking of the vessel, bubbling via gas introduction, and/or manual agitation of mycelium clumps. In some examples, a non-reactive additive, such as glass beads, can be added to facilitate interruption of mycelium clumps. Agitation parameters, such as type, speed, sequencing, etc. can be selected based on the specific fungal species. Exemplary agitator speeds include 100 rpm or greater, 200 rpm or greater, 300 rpm or greater, 400 rpm or greater, 500 rpm or greater, or 600 rpm or greater. In some examples, the mixture is aerated during at least a portion of the incubation period. An exemplary aeration rate is 1 liter of gas per 1 liter of medium per minute (VVM). The aeration gas may be air, nitrogen, and/or oxygen.

In an exemplary embodiment, the incubation step 106 can include agitating the mixture according to a predetermined agitation profile. Without wishing to be limited by any theory, it is believed that if the initial agitation within the reaction vessel at the beginning of the incubation step 106 is too high, the growth rate of the mycelia and/or the morphology of the mycelia may be affected. An initial agitation rate that is too high may affect the ability of the liquid grow process to produce a desired amount of mycelia having the desired morphological characteristics within a predetermined period of time (e.g., a minimum length of hyphae branches, a desired density of the hyphae network, a desired degree of branching of the hyphae, a desired aspect ratio, and/or a desired degree of hyphal fusion of the hyphae network). For example, a high agitation rate in the initial stages of step 106 may result in ejection of the spores and/or early mycelia onto the walls or other components within the reaction vessel, which may impact the overall growth rate and/or morphology of the mycelia. Conversely, it has been found that if the agitation rate is too low, dissolved oxygen levels may not remain high enough, which can also affect the growth rate and/or morphology of the mycelia.

In some aspects, the incubation step 106 can include an agitation profile that includes an initial stage having a low agitation rate (e.g., 200 rpm) for a predetermined period of time, followed by a ramp stage during which the agitation rate is increased. Optionally, the agitation profile can include maintaining the agitation rate at a predetermined high rate following the ramp stage, greater than the initial agitation rate (e.g., 1000 rpm), until the end of the incubation step 106. Parameters of the ramp stage, such as the initiation time of the ramp stage, the rate of increase of agitation, and/or the end of the ramp stage may be determined at least in part based on real-time data from the reaction vessel and/or experimental data. In one aspect, the ramp stage may include a continuous ramp profile in which the agitation rate increases continuously from the initial stage to an end of the ramp stage. In another aspect, the ramp stage may include a discontinuous ramp profile in which the agitation rate increases from the initial stage to the end of the ramp stage, but may include intermittent periods in which the agitation rate at least partially decreases. For example, the dissolved oxygen level within the reaction vessel can be monitored and one or more parameters of the ramp stage can be adjusted to maintain the dissolved oxygen level above a predetermined minimum value and/or within a predetermined range of oxygen levels. In some aspects, the ramp stage can be configured to increase the agitation rate to maintain the level of dissolved oxygen above a predetermined minimum value, such as about 20% or greater. For example, the agitation profile can include agitating the mixture at a low agitation rate in the initial stage for a predetermined period of time, followed by a ramp stage that is configured to increase the agitation rate to maintain the level of dissolved oxygen at or above a predetermined level, such as, for example, at about 20% or greater, about 22% or greater, about 24% or greater, about 26% or greater, about 28% or greater, or about 30% or greater. The desired minimum dissolved oxygen level may vary depending on fungal species.

Without wishing to be limited by any theory, it is believed that as the incubation step 106 progresses and the mycelia grow, the viscosity of the mixture in the vessel may increase. An increase in agitation (e.g., an increase in agitator speed) as the viscosity of the mixture increases may facilitate sufficient oxygen delivery to the mycelia to support growth of mycelium having the desired morphological characteristics. However, when the degree of agitation is too high (e.g., high agitation rates) the proportion of mycelium having the desired morphological characteristics, such as a desired length, dispersion, and/or degree of branching of the hyphae, may decrease. The effect of high agitation rates may become more pronounced as the volume of the mixture increases. For example, in a 2 L bioreactor vessel, agitator speeds of 1000 rpm and greater may result in a decrease in the production of long filaments and an increase in the proportion of mycelia having shorter filament fragments (e.g., fragment lengths <200 μm). However, as discussed above, if the agitator speeds are too low, particularly at later stages in the growth process, the growth rate and/or morphological characteristics of the mycelia biomass may be affected. Implementation of the agitation profile according to the present disclosure can facilitate promoting the growth of a mycelia biomass having the desired morphological characteristics within a predetermined period of time. It is understood that suitable agitator speeds and parameters of the agitation profile may be scaled accordingly based on characteristics of the mixture (e.g., viscosity), the type of agitator used and/or the dimensions of the reactor vessel.

Agitation during the incubation step 106 may be achieved using any suitable agitator or combination of agitators. For example, the agitator may be an impeller type, such as for example a Rushton turbine impeller. The size, shape, type, and number of agitators may be based at least in part on the dimensions of the reactor vessel and/or the shear forces encountered during the incubation step 106.

The incubation step 106 is configured to promote the growth of a biomass of mycelium that includes a plurality of branches of hyphae. The cultivated biomass of mycelium forms a slurry including mycelium suspended within the mixture. Some mycelium may also grow on components of the bioreactor, such as the walls of the vessel, the agitator or impeller (if present), and/or other components that the mixture is in contact with inside the bioreactor. The incubation step 106 can be ended when the cultivated biomass of mycelium is collected at step 108. The incubation step 106 may be ended at a predetermined time or when a predetermined concentration of mycelium biomass is reached. There may be some continued growth of the mycelium after the cultivated biomass is collected at step 108. Optionally, the mycelium biomass may be treated to stop growth of the mycelium. In some aspects, the incubation step 106 may last for about 24-72 hours. For example, the incubation step 106 may last for at least about 24 hours, at least about 48 hours, at least about 72 hours, or any time period between these values.

At step 108 a concentration of the collected biomass of mycelium may be adjusted based on the subsequent web-forming process at step 110. In some examples, the cultivated biomass of mycelium is in the form of a slurry. The concentration of the biomass of mycelium may be adjusted by increasing a volume of the slurry or concentrating the mycelium biomass by removing at least a portion of the liquid from the slurry. In some examples, the concentration of the mycelium biomass may be adjusted to a concentration of from about 10 g/L to about 30 g/L, about 10 g/L to about 25 g/L, about 10 g/L to about 20 g/L, about 10 g/L to about 15 g/L, about 12 g/L to about 30 g/L, about 12 g/L to about 25 g/L, about 12 g/L to about 20 g/L, about 12 g/L to about 15 g/L, about 15 g/L to about 30 g/L, about 15 g/L to about 25 g/L, or about 15 g/L to about 20 g/L. In other examples, the cultivated biomass of mycelium may be collected and dried.

In some aspects, at step 114, a bonding agent can optionally be added to the cultivated biomass of mycelium before, during, or after the web-forming process at step 110. The bonding agent can be added before, during, or after collecting the cultivated biomass of mycelium and/or adjusting the concentration of the cultivated biomass of mycelium. The bonding agent can include any adhesive, resin, cross-linking agent, or polymeric matrix material described herein and combinations thereof.

In some aspects, the plurality of branches of hyphae can optionally be disrupted at step 114, before, during, or after the web-forming process at step 110. The plurality of branches of hyphae can be disrupted according to any of the mechanical and/or chemical methods described herein for disrupting hyphae. For example, prior to the web-forming process at step 110, the hyphae can mechanically disrupted by a mechanical action such as blending, chopping, impacting, compacting, bounding, shredding, grinding, compressing, high-pressure waterjet, or shearing forces. The hyphae can be disrupted before, during, or after adjusting the concentration of the cultivated biomass of mycelium.

In some aspects, the collected biomass of mycelium can optionally be combined with natural and/or synthetic fibers at step 114, before, during, or after the web-forming process at step 110. In one aspect, the fibers can be combined with the mycelium before, during, or after disrupting the plurality of branches of hyphae. The fibers can have any suitable dimension. Non-limiting examples of suitable fibers include cellulosic fibers, cotton fibers, rayon fibers, Lyocell fibers, TENCEL™ fibers, polypropylene fibers, and combinations thereof. In one aspect, the fibers can have a length of less than about 25 mm, less than about 20 mm, less than about 15 mm, or less than about 10 mm. For example, the fibers can have a length of from about 1 mm to about 25 mm, about 1 mm to about 20 mm, about 1 mm to about 15 mm, about 1 mm to about 10 mm, about 1 mm to about 5 mm, about 5 mm to about 25 mm, about 5 mm to about 20 mm, about 5 mm to about 15 mm, about 5 mm to about 10 mm, about 10 mm to about 25 mm, about 10 mm to about 20 mm, or about 10 mm to about 15 mm. The fibers may be combined with the mycelium in a desired concentration. In one example, the fibers may be combined with the mycelium in an amount of from about 1 wt % to about 25 wt %, about 1 wt % to about 20 wt %, about 1 wt % to about 15 wt %, about 1 wt % to about 10 wt %, about 1 wt % to about 5 wt %, about 5 wt % to about 25 wt %, about 5 wt % to about 20 wt %, about 5 wt % to about 15 wt %, about 5 wt % to about 10 wt %, about 10 wt % to about 25 wt %, about 10 wt % to about 20 wt %, or about 10 wt % to about 15 wt %.

In some aspects, the collected biomass of mycelium can optionally by combined with a reinforcing material at step 114, before, during, or after the web-forming process at step 110. As described herein, in some aspects the supporting material is a reinforcing material. Non-limiting examples of a suitable supporting material include a woven fiber, a mass of contiguous, disordered fibers (e.g., non-woven fibers), perforated material (e.g., a metal mesh or perforated plastic), a mass of discontiguous particles (e.g., pieces of woodchip), a cheesecloth, a fabric, a knot fiber, a scrim, and a textile. The hyphae can be combined with, contacted with, and/or incorporated into the supporting material. For example, in some aspects, the hyphae can be woven, twisted, would, folded, entwined, entangled, and/or braided together with the supporting material to form a mycelium material, as described herein. In some aspects, the fibers can be laid on the supporting material before, during, and/or after adding a chemical bonding agent.

At step 110, the biomass of mycelium collected in step 108 can be treated according to a web-forming process to form a hyphal network. The web-forming process can include any of the wet-lay, dry array, or air-lay techniques described herein. The hyphae of the web formed in step 110 can optionally be chemically and/or thermally bonded using any of the bonding agents described herein. Optionally, the web-forming at step 110 can include laying the branches of hyphae on a supporting material.

At optional step 112, the hyphal network formed at step 110 can undergo an entanglement process to entangle the plurality of branches of hyphae in the hyphal network. The entanglement process can include needle punching (also referred to as felting) and/or hydroentangling. When a supporting material is present, the entanglement process optionally includes entangling at least a portion of the plurality of hyphae branches with the supporting material. The entanglement process can form mechanical interactions between hyphae and optionally between hyphae and a supporting material (when present). In some embodiments, the hyphae are not entangled with a supporting material.

In some aspects, the entanglement at step 112 is achieved through a needle punching or needle felting process in which one or more needles are passed into and out of the hyphal network. Movement of the needles in and out of the hyphal network facilitate entangling the hyphae and optionally orienting the hyphae. A needle punch having an array of needles can be used to punch the hyphal network at a plurality of locations with each pass of the needle array. The number of needles, spacing of needles, shape of the needles, and size of the needles (i.e., needle gauge) can be selected to provide the desired degree of entanglement of the hyphal network. For example, the needles may be barbed and have any suitable shape, non-limiting examples of which include a pinch blade, a star blade, and a conical blade. The number of needle punches per area and the punching rate can also be selected to provide the desired degree of entanglement of the hyphal network. The parameters of the needle punching or needle felting process can be selected at least based in part on the fungal species, the morphology and dimensions of the hyphae forming the hyphal network, the desired degree of entanglement, and/or end-use applications of the mycelium material.

In some aspects, the entanglement at step 112 is achieved through a hydroentanglement process. The hydroentanglement process directs high pressure liquid jets into the hyphal network to facilitate entangling the hyphae. The liquid may be any suitable liquid, an example of which includes water. The entanglement process can include a spinneret having an array of holes configured to direct a stream of liquid at a specific location in the hyphal network. The diameter of the holes can be selected to provide a jet of liquid having the desired diameter to direct at the hyphal network. Additional aspects of the spinneret, such as the number of holes in the array and the spacing of the holes in the array can be selected to provide the desired degree of entanglement of the hyphal network. The hyphal network and the spinneret may move relative to one another such that the liquid jets are directed at the hyphal network in a pattern. For example, the spinneret may move relative to the hyphal network in a generally “Z” or “N” shaped pattern to provide multiple passes of the spinneret over the hyphal network. The number of passes and the application pattern can be selected to provide the desired degree of entanglement of the hyphal network. The parameters of the hydroentanglement process can be selected based at least in part on the fungal species, the morphology and dimensions of the hyphae forming the hyphal network, the desired degree of entanglement, and/or end-use applications of the mycelium material. In some examples, the hydroentanglement process occurs in phases in which a portion of the mycelium material is web-formed (e.g., wet-laying), the hydroentanglement process proceeds, and then a second portion of the mycelium material is web-formed on top of the first portion and the hydroentanglement process is repeated. This process of web-forming a portion of the mycelium material and hydroentangling the web-formed portion can be repeated any number of times until a final thickness of material is web-formed.

The liquid pressure, the diameter of the openings in the spinneret, and/or the flow rate of liquid can be selected to provide the desired degree of entanglement of the hyphal network and optionally entanglement of the hyphal network and a supporting material. For example, the liquid pressure during the hydroentanglement process can be at least 100 psi, at least 200 psi, at least 300 psi, at least 400 psi, at least 500 psi, at least 600 psi, at least 700 psi, at least 800 psi, at least 900 psi, or at least 1000 psi. In some examples, the liquid jet pressure is from about 700 to about 900 psi. In some examples, the diameter of the openings in the spinneret is at least about 10 microns, at least about 30 microns, at least about 50 microns, at least about 70 microns, at least about 90 microns, at least about 110 microns, at least about 130 microns, or at least about 150 microns. For example, the diameter of the openings in the spinneret can be from about 10 microns to about 150 microns, from 20 microns to about 70 microns, about 30 microns to about 80 microns, about 40 microns to about 90 microns, about 50 microns to about 100 microns, about 60 microns to about 110 microns, or about 70 microns to about 120 microns. In some examples, the openings have a diameter of about 50 microns. The flow rate of liquid can be from about 100 mL/min. to about 300 mL/min. in some examples. In some examples, the belt speed during the entanglement process is about 1 meter/minute.

After completion of the entanglement process at 112, the mycelium material can be processed according to any of the post-processing methods and/or treatments described herein. Non-limiting examples of post-processing methods and treatments include treatment with a plasticizer, treatment with a tannin and/or dye, treatment with a preservative, treatment with a protein source, treatment with a coating and/or finishing agent, a drying process, a rolling or flattening process, and treatment in an embossing process.

The mycelium biomass may be treated according to a drying process at one or more stages following collection of the biomass at 108. The drying process can include an air drying process (i.e., drying without the application of heat), a thermal drying process (i.e., drying with the application of heat), a vacuum drying process (i.e., removing water via application of a vacuum), and/or a press-drying process (i.e., removing liquid via the application of a pressing or squeezing force).

In various embodiments, the liquid or solid substrate may be supplemented with one or more different nutritional sources. The nutritional sources may contain lignocellulose, simple sugars (e.g. dextrose, glucose), complex sugars, agar, malt extract, a nitrogen source (e.g. ammonium nitrate, ammonium chloride, amino acids) and other minerals (e.g. magnesium sulfate, phosphate). In some embodiments, one or more of the nutritional sources may be present in lumber waste (e.g. sawdust) and/or agricultural waste (e.g. livestock feces, straw, corn stover). Once the substrate has been inoculated and, optionally, supplemented with one or more different nutritional sources, cultivated mycelium may be grown. Methods of growing mycelium have been well established in the art. Exemplary methods of growing mycelium include but are not limited to U.S. Pat. Nos. 5,854,056; 4,960,413; and 7,951,388.

In some embodiments, the growth of the cultivated mycelium will be controlled to prevent the formation of fruiting bodies. Various methods of preventing fruiting body formation as discussed in detail in U.S. Patent Publication No. 2015/0033620; U.S. Pat. Nos. 9,867,337; and 7,951,388. In other embodiments, the cultivated mycelium may be grown so that it is devoid of any morphological or structural variations. Depending on the embodiment sought, growing conditions such as exposure to light (e.g. sunlight or a growing lamp), temperature, carbon dioxide may be controlled during growth.

In some embodiments, the cultivated mycelium may be grown on an agar medium. Nutrients may be added to the agar/water base. Standard agar media commonly used to cultivate mycelium material include, but are not limited to, a fortified version of Malt Extract Agar (MEA), Potato Dextrose Agar (PDA), Oatmeal Agar (OMA), and Dog Food Agar (DFA).

In most embodiments, the cultivated mycelium material may be grown as a solid mass and may later be disrupted. Cultivated mycelium material that is disrupted may be a live mat, preserved, or otherwise treated to kill the mycelium (i.e., stop mycelium growth) as described below.

In some embodiments, cultivated mycelium material may be grown to include elongate hyphae defining fine filaments that interconnect with one another, and further may interconnect with various supporting materials provided in a growing procedure, as further described below. The fine filaments may be analyzed using an optical magnifying or imaging device to determine if a grown length of the fine filaments is adequate to support sufficient network interconnection between the fine filaments and various additives. The fine filaments should not only be of a sufficient length, but also flexible to provide adequate interconnection therebetween.

In some embodiments, cultivated mycelium material may be processed using a dry array, a wet-lay, or an air-lay technique. In dry-lay or dry array, an inert or growing mycelium network of branched hyphae may be pulled apart and detangled to expand the volume of the network. Similarly, in a wet-lay technique, an inert or growing mycelium network of branched hyphae may be saturated in a liquid medium to detangle and expand the volume of the network. Further, in an air-lay technique, an inert or growing mycelium network of branched hyphae may be suspended in air to create a web that expands the volume of the network. After such a technique, the expanded network can be compressed to provide a dense or compacted network. The web can be densified to include an overall density profile of 6 gm per cubic meter. A compacted web can be embossed with a replicated leather pattern for providing a leather alternative material.

### Disrupted Cultivated Mycelium Material

Various types of cultivated mycelium material including one or more masses of branching hyphae may be disrupted at a variety of points during the production process, thus generating one or more masses of disrupted branching hyphae. In such embodiments, the cultivated mycelium material comprises one or more masses of disrupted branching hyphae. The cultivated mycelium material may be disrupted before or after adding a bonding agent. In one aspect, the cultivated mycelium material may be disrupted at the same time as adding a bonding agent. Exemplary embodiments of disruptions include, but are not limited to, mechanical action, chemical treatment, or a combination thereof. For example, the one or more masses of branching hyphae may be disrupted by both a mechanical action and chemical treatment, a mechanical action alone, or chemical treatment alone.

In some embodiments, the one or more masses of branching hyphae is disrupted by a mechanical action. Mechanical actions may include blending, chopping, impacting, compacting, bounding, shredding, grinding, compressing, high-pressure, waterjet, and shearing forces. In some embodiments, the mechanical action includes blending the one or more masses of branching hyphae. Exemplary methods of achieving such a disruption include use of a blender, a mill, a hammer mill, a drum carder, heat, pressure, liquid such as water, a grinder, and a beater. In an exemplary production process, a cultivated mycelium material is mechanically disrupted by a conventional unit operation, such as homogenization, grinding, coacervation, milling, jet milling, waterjet and the like.

According to a further aspect, the mechanical action includes applying a physical force to the one or more masses of branching hyphae such that at least some of the masses of branching hyphae are aligned in a particular formation, e.g., aligned in a parallel formation, or along or against the stress direction. The physical force can be applied to one or more layers of a cultivated mycelium material or composite mycelium material. Such disrupted mycelia material can typically be constructed with layers with varying orientation. Exemplary physical forces include, but are not limited to, pulling and aligning forces. Exemplary methods of achieving such a disruption include use of rollers and drafting equipment. In some embodiments, a physical force is applied in one or more directions such that the at least some of the masses of branching hyphae are aligned in parallel in one or more directions, wherein the physical force is applied repeatedly. In such embodiments, the physical force may be applied at least two times, e.g., at least three times, at least four times, or at least five times.

In some other embodiments, the one or more masses of branching hyphae is disrupted by chemical treatment. In such embodiments, the chemical treatment includes contacting the one or more masses of branching hyphae with a base or other chemical agent sufficient to cause a disruption including, but not limited to alkaline peroxide, beta-glucanase, surfactants, and bases such as sodium hydroxide and sodium carbonate. The pH of the cultivated mycelium material in solution can be monitored for the purpose of maintaining the optimal pH.

In some embodiments, the disruptions described herein generate one or more masses of disrupted branching hyphae, e.g., sub-networks. As used herein, a “sub-network” refers to discrete masses of branching hyphae that are produced after disruption, e.g., a mechanical action or chemical treatment. A sub-network may come in a wide assortment of shapes, e.g., sphere-, square-, rectangular-, diamond-, and odd-shaped sub-networks, etc., and each sub-network may come in varied sizes. The cultivated mycelium material may be disrupted sufficiently to produce one or more masses of disrupted branching hyphae, e.g., sub-networks, having a size in the desired ranges. In many instances, the disruption can be controlled sufficiently to obtain both the size and size distribution of the sub-network within a desired range. In other embodiments, where more precise size distributions of sub-networks are required, the disrupted cultivated mycelium material can be further treated or selected to provide the desired size distribution, e.g. by sieving, aggregation, or the like. For example, a sub-network may have a size represented by, e.g., length, of about 0.1 mm to about 5 mm, inclusive, e.g., of about 0.1 mm to about 2 mm, about 1 mm to about 3 mm, about 2 mm to about 4 mm, and about 3 mm to about 5 mm. In some embodiments, a sub-network may have a size represented by a length of about 2 mm. The “length” of a sub-network is a measure of distance equivalent to the most extended dimension of the sub-network. Other measurable dimensions include, but are not limited to, length, width, height, area, and volume.

In various embodiments, physical force may be used to create new physical interactions (i.e. re-entangle) between the one or more masses of branching hyphae after disruption. Various known methods of creating entanglements between fiber may be used, including methods of creating non-woven materials by creating mechanical interactions between fibers. In some embodiments described below, hydroentanglement may be used to create mechanical interactions between the hyphae after the hyphae have disrupted.

### Preserved Cultivated Mycelium Material

Once the cultivated mycelium material has been grown, it may be optionally separated from the substrate in any manner known in the art, and optionally subjected to post-processing in order to prevent further growth by killing the mycelium and otherwise rendering the mycelium imputrescible, referred to herein as “preserved mycelium material”. Suitable methods of generating preserved mycelium material can include drying or desiccating the cultivated mycelium material (e.g. pressing the cultivated mycelium material to expel moisture) and/or heat treating the cultivated mycelium material. In a specific embodiment, the cultivated mycelium material is pressed at 190,000 pounds force to 0.25 inches for 30 minutes. Suitable methods of drying organic matter to render it imputrescible are well known in the art. In one specific embodiment, the cultivated mycelium material is dried in an oven at a temperature of 100° F. or higher. In another specific embodiment, the cultivated mycelium material is heat pressed.

In other instances, living or dried cultivated mycelium material is processed using one or more solutions that function to remove waste material and water from the mycelium. In some embodiments, the solutions include a solvent such as ethanol, methanol or isopropyl alcohol. In some embodiments, the solutions include a salt such as calcium chloride. Depending on the embodiments, the cultivated mycelium material may be submerged in the solution for various durations of time with and without pressure. In some embodiments the cultivated mycelium material may be submerged in several solutions consecutively. In a specific embodiment, the cultivated mycelium material may first be submerged in one or more first solutions including an alcohol and a salt, then submerged in a second solution including alcohol. In another specific embodiment, the cultivated mycelium material may first be submerged in one or more first solutions including an alcohol and a salt, then submerged in a second solution including water. After treatment with solution, the cultivated mycelium material may be pressed using a hot or cold process and/or dried using various methods including air drying and/or vacuum drying. U.S. Patent Publication No. 2018/0282529, the entirety of which is incorporated herein by reference, describes these embodiments in detail.

In one aspect, the cultivated mycelium material may be fixated by adjusting pH using an acid such as formic acid. In specific embodiments, the pH will be at least 2, 3, 4 or 5. In some embodiments, the pH of the cultivated mycelium material will be adjusted to an acidic pH of 3 in order to fix the cultivated mycelium material using various agents such as formic acid. In specific embodiments, the pH will be adjusted to a pH less than 6, 5, 4 or 3 in order to fix the cultivated mycelium material. In one embodiment, the pH will be adjusted to a pH of 5.5.

### Bonding Agents

Various aspects of the present disclosure include a bonding agent. A “bonding agent” as used herein may include any suitable agent that provides added strength and/or other properties such as additional softness, strength, durability, and compatibility. A bonding agent may be an agent that reacts with some portion of the cultivated mycelium material, enhances the treatment of the cultivated mycelium material, co-treated with the cultivated mycelium material or treated separately, but as a network with the cultivated mycelium material, to produce a composite mycelium material. In some aspects, a bonding agent is added prior to the disruption. In other aspects, a bonding agent is added after the disruption. In some other aspects, a bonding agent is added while the sample is being disrupted. Bonding agents include an adhesive, a resin, a crosslinking agent, and/or a matrix. A composite mycelium material described herein includes cultivated mycelium material and bonding agents that may be water-based, 100% solids, UV and moisture cure, two-component reactive blend, pressure sensitive, self-crosslinking hot melt, and the like.

In some embodiments, the bonding agent is selected from the group including a natural adhesive or a synthetic adhesive. In such embodiments, the natural adhesive may include a natural latex-based adhesive. In specific embodiments, the natural latex-based adhesive is leather glue or weld. The bonding agents may include anionic, cationic, and/or non-ionic agents. In one aspect, the bonding agents may include crosslinking agents.

In some embodiments, the bonding agent has a particle size of less than or equal to 1 μm, a sub-zero glass transition temperature, or a self-crosslinking function. In some embodiments, the bonding agent has a particle size of less than or equal to 1 μm, a sub-zero glass transition temperature, and a self-crosslinking function. In some embodiments, the bonding agent has a particle size of less than or equal to 1 μm. In some embodiments, the bonding agent has a sub-zero glass transition temperature. In some embodiments, the bonding agent has a self-crosslinking function. In some embodiments, the bonding agent has a particle size of less than or equal to 500 nanometers. Specific exemplary bonding agents include vinyl acetate ethylene copolymers such as Dur-O-Set® Elite Plus and Dur-O-Set® Elite 22.

In some embodiments, the bonding agent has a glass transition temperature of about −100° C. to −10° C., −100° C. to −90° C., −90° C. to −80° C., −80° C. to −70° C., −70° C. to −60° C., −60° C. to −50° C., −50° C. to −40° C., −40° C. to −30° C., −30° C. to −20° C., −20° C. to −10° C., −10° C. to 0° C., −30° C. to −25° C., −25° C. to −20° C., −20° C. to −15° C., −15° C. to −10° C., −10° C. to −5° C., −5° C. to 0° C., about −90° C., about −80° C., about −70° C., about −60° C., about −50° C., about −40° C., about −35° C., about −30° C., about −25° C., about −20° C., about −15° C., about −10° C., about −5° C., about or 0° C. In some embodiments, the bonding agent has a glass transition temperature of about −15° C.

Other exemplary bonding agents include, but are not limited to transglutaminase, polyamide-epichlorohydrin resin (PAE), citric acid, genipin, and alginate. In some embodiments, the bonding agent includes one or more reactive groups. For example, the bonding agent reacts with active hydrogen containing groups such as amine, hydroxyl, and carboxyl groups. In a specific embodiment, the bonding agent crosslinks one or more masses of branching hyphae via the one or more reactive groups. In some instances, amines are present on chitin, and hydroxyl and carboxyl groups are present on the polysaccharides and proteins surrounding the chitin. In a specific embodiment, PAE includes cationic azetidinium groups. In such embodiments, the cationic azetidinium groups on PAE act as reactive sites in the polyamideamine backbone, and react with active hydrogen containing groups such as amine, hydroxyl, and carboxyl groups, in the one or more branches of hyphae.

Further examples of bonding agents include, but are not limited to, citric acid in combination with sodium hypophosphite or monosodium phosphate or sodium dichloroacetate, alginate in combination with sodium hypophosphite or monosodium phosphate or sodium dichloroacetate, epoxidized soybean oil, and N-(3-Dimethylaminopropyl)-N′-ethylcarbodiimide hydrochloride (EDC). Some examples of bonding agents include epoxies, isocyanates, sulfur compounds, aldehydes, anhydrides, silanes, aziridines, and azetidinium compounds and compounds with all such functional groups. Possible formaldehyde-containing bonding agents include formaldehyde, phenol formaldehyde, urea formaldehyde, melamine urea formaldehyde, melamine formaldehyde, phenol resorcinol and any combinations of them.

Additional examples of suitable bonding agents include latex materials, such as butadiene copolymers, acrylates, vinyl-acrylics, styrene-acrylics, styrene-butadiene, nitrile-butadiene, polyvinyl acetates, olefin containing polymers, e.g., vinyl acetate-ethylene copolymers, vinyl ester copolymers, halogenated copolymers, e.g., vinylidene chloride polymers. Latex-based agents, when used, can contain functionality. Any kind of latex can be used, including acrylics. Representative acrylics include those formed from ethyl acrylate, butyl acrylate methyl (meth)acrylate, carboxylated versions thereof, glycidylated versions thereof, self-crosslinking versions thereof (for example, those including N-methyl acrylamide), and copolymers and blends thereof, including copolymers with other monomers such as acrylonitrile. Natural polymers such as starch, natural rubber latex, dextrin, lignin, cellulosic polymers, saccharide gums, and the like can also be used. In addition, other synthetic polymers, such as epoxies, urethanes, phenolics, neoprene, butyl rubber, polyolefins, polyamides, polypropylene, polyesters, polyvinyl alcohol, and polyester amides can also be used. The term “polypropylene” as used herein includes polymers of propylene or polymerizing propylene with other aliphatic polyolefins, such as ethylene, 1-butene, 1-pentene, 3-methyl-1-butene, 4-methyl-1-pentene, 4-methyl-1-hexene, 5-methyl-1-hexene and mixtures thereof. In specific embodiments, bonding agents include, but are not limited to, natural adhesives (e.g. natural latex-based adhesives such as leather glue or weld, latex, soy protein-based adhesives), synthetic adhesives (polyurethane), neoprene (PCP), acrylic copolymer, styrene-butadiene copolymer, ethylene-vinyl acetate-b, nitrocellulose, and polyvinyl acetate (PVA).

In one aspect, one or more bonding agents may be incorporated within the cultivated mycelium material to be bonded, either in its disrupted or undisrupted state, e.g., embedded throughout the material, or added as a thin coating layer, such as by spraying, dipping, rolling, coating, and the like, to produce a composite mycelium material. In one other aspect, one or more bonding agents may be incorporated at the same time the disruption occurs. Any suitable method of bonding may be used according to the present disclosure. Bonding of the surfaces may occur on drying, and a strong cured bond can be developed. The bonding of one or more bonding agents may include the use of open or closed-cell foam materials like urethane, olefinic rubber, and vinyl foam materials, as well as textiles, metal and fabrics in various lamination arrangements.

A bonded assembly (i.e., a laminate) may be prepared by uniformly applying the aqueous adhesive to the cultivated mycelium material. In some embodiments, the lamina includes two successive layers. In some embodiments, the lamina includes three successive layers. Various coating methods may be used such as spraying, roll coating, saturation, and the like. The coated substrate can be dried before bonding.

A composite mycelium material may be chemically bonded by impregnating the composite mycelium material with a chemical binder to link fibers to one another, including linking cellulosic fibers to one another. Non-limiting examples of suitable binders include gum arabic, vinyl acetate-ethylene (VAE), and adhesives. Examples of suitable adhesive include S-10, available from USAdhesives, U.S.A., and Bish's Original Tear Mender Instant Fabric & Leather Adhesive, available from Tear Mender, U.S.A. One example of a suitable VAE-based binder is Dur-O-Set® Elite 22, which is available from Celanese Emulsions, U.S.A. Another exemplary binder includes X-LINK® 2833, available from Celanese Emulsions, U.S.A., and which is described as a self-crosslinking vinyl acrylic. In a web of interconnected hyphae, a chemical binder will have to saturate the web to diffuse through the web and reach the core of the network. Thus, a composite mycelium material may be immersed in a binder solution to fully impregnate the material. A spray application of a chemical binder may also be provided to a composite mycelium material. A spray application of a chemical binder may be aided by capillary action for disbursement, or may be aided by a vacuum application to draw the chemical binder through the material. A coater may also be used for coating a composite mycelium material.

A composite mycelium material may be bonded using a thermal bonding technique, wherein an additive is provided along with the composite mycelium material. This additive may be a “meltable” material that melts at a known heat level. The cellulosic material of the composite mycelium material does not melt, such that the composite mycelium material along with the additive can be heated to the additive's melting point. As melted, the additive can disperse within the composite mycelium material and then be cooled to harden the overall material.

The present disclosure is not limited to the above lists of suitable bonding agents. Other bonding agents are known in the art. The role of a bonding agent, regardless of type, is to, in part, provide several reactive sites per molecule. The type and amount of bonding agent used in the present disclosure depend on what properties are desired. In various embodiments, an effective amount of bonding agent may be used. As used herein, an “effective amount” with respect to a bonding agent refers to the amount of agent that is sufficient to provide added strength and/or other properties such as additional softness, strength, durability, and compatibility.

### Supporting Materials

According to one aspect, the cultivated mycelium material or composite mycelium material may further include a supporting material, e.g., to form a bonded assembly, i.e., a laminate. As used herein, the term “supporting material” refers to any material, or combination of one or more materials, that provide support to the cultivated mycelium material or composite mycelium material.

In some embodiments, the supporting material is entangled within the cultivated mycelium material or composite mycelium material, e.g., a reinforcing material. In some other embodiments, the supporting material is positioned on a surface of the cultivated mycelium material or composite mycelium material, e.g., a base material. In some embodiments, the supporting material includes, but is not limited to, a mesh, a cheesecloth, a fabric, a knit fiber, a woven fiber, and a non-woven fiber. In some embodiments, the supporting material may be constructed in whole or in part of any combination of synthetic fiber, natural fiber (e.g. lignocellulosic fiber), metal, or plastic. The supporting material may be entangled, in part, within the cultivated mycelium material or composite mycelium material, e.g., using known methods of entanglement like felting or needle punching. In some aspects, the supporting material is not entangled within the cultivated mycelium material or composite mycelium material. Various methods known in the art may be used to form a laminate as described herein. In some other embodiments, the supporting material includes a base material that is, e.g., applied to a top or bottom surface of a cultivated mycelium material or composite mycelium material. The supporting material may be attached through any means known in the art, including, but not limited to, chemical attachment, e.g., a suitable spray coating material, in particular, a suitable adhesive, or alternatively, e.g., due to their inherent tackiness.

A laminate according to the present disclosure may include at least one supporting material. If more than one supporting material is used, the cultivated mycelium material or composite mycelium material can include an inner layer of a sandwich of multiple layers, with the inner layer, e.g., being a supporting material such as a knit or woven or scaffold. In this instance, the supporting material would be embedded within the cultivated mycelium material or composite mycelium material.

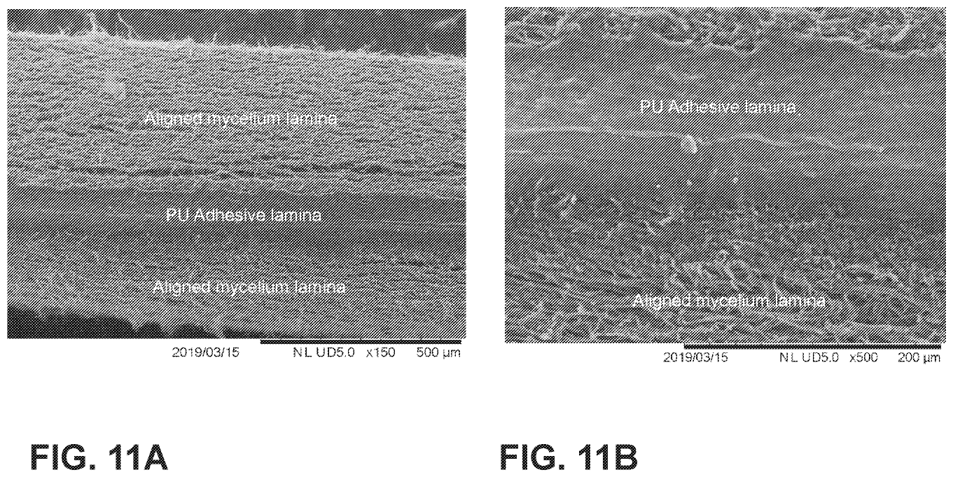

Supporting materials as used herein can include scaffolds or textiles. A “scaffold” as used herein refers to any material known in the art that is distinct from the cultivated mycelium material and provides support to the cultivated mycelium material or composite mycelium material. A “scaffold” may be embedded within the cultivated mycelium material or composite mycelium material or layered on, under, or within the cultivated mycelium material or composite mycelium material. In the present disclosure, all kinds and types of scaffolds may be used, including, but not limited to films, textiles, scrims, and polymers. A “textile” as used herein refers to a type of scaffold that may be any woven, knitted, or non-woven fibrous structure. Where multiple layers are included in the cultivated mycelium material or composite mycelium material, such as shown in FIGS. 11A and 11B, the two or more layers may include a scaffold; or in other embodiments, the two or more layers may include a cheesecloth. Useful scaffolds include woven and non-woven scaffolds, directional and non-directional scaffolds, and orthogonal and non-orthogonal scaffolds. Useful scaffolds may include conventional scaffolds, which include a plurality of yarns oriented in the machine direction, or along the length of the scaffold, and a plurality of yarns oriented in the cross-machine direction, or across the width of the scaffold. These yarns may be referred to as the warp yarns and weft yarns, respectively. Numerous yarns can be employed including, but not limited to, fibrous materials and polymers. For example, the yarns can include, but are not limited to, fiberglass, aluminum, or aromatic polyamide polymers. In one embodiment, the scaffold includes fiberglass yarns. The scaffolds may be adhered together or locked into position using conventional bonding agents such as cross-linkable acrylic resins, polyvinyl alcohol, or similar adhesives. The scaffolds may also be mechanically entangled by employing techniques such as, but not limited to, needle punching. In yet another embodiment, the scaffolds can be locked into place by weaving. A combination of supporting materials may be used according to the present disclosure.

In some embodiments, supporting materials may be incorporated into a cultivated mycelium material or composite mycelium material as described herein according to methods known in the art, including but not limited to the methods described in U.S. Pat. Nos. 4,939,016 and 6,942,711, the entirety of which are incorporated herein by reference. For example, supporting materials may be incorporated into a cultivated mycelium material or composite mycelium material via hydroentanglement. In such embodiments, supporting materials may be incorporated into a cultivated mycelium material or composite mycelium material before or after adding a bonding agent and/or a crosslinking agent. In some embodiments, a liquid such as water directed to the cultivated mycelium material or composite mycelium material through one or more pores for hydroentanglement can pass through the cultivated mycelium material or composite mycelium material. In some embodiments, the liquid is a high-pressure liquid. In some embodiments, the pressure and water flow may vary depending, in part, on the type of supporting material and pore size. In various embodiments, the water pressure is at least 100 psi, e.g., at least 200 psi, at least 300 psi, at least 400 psi, at least 500 psi, at least 600 psi, at least 700 psi, at least 800 psi, at least 900 psi, and at least 1000 psi. In various embodiments, the water pressure is about 100 psi to about 5000 psi, inclusive, e.g., about 200 psi to about 1000 psi, about 300 psi to about 2000 psi, about 400 psi to about 3000 psi, about 500 psi to about 4000 psi, and about 600 psi to about 5000 psi. In some embodiments, the water pressure is about 750 psi. In various embodiments, the one or more pores has a diameter of at least 10 microns, e.g., at least 30 microns, at least 50 microns, at least 70 microns, at least 90 microns, at least 110 microns, at least 130 microns, and at least 150 microns. In various embodiments, the one or more pores has a diameter of about 10 microns to about 150 microns, inclusive, e.g., about 20 microns to about 70 microns, about 30 microns to about 80 microns, about 40 microns to about 90 microns, about 50 microns to about 100 microns, about 60 microns to about 110 microns, and about 70 microns to about 120 microns. In some embodiments, the one or more pores has a diameter of about 50 microns.

The cultivated mycelium material or composite mycelium material may also include auxiliary agents that are used in foam materials. Auxiliary agents or additives include crosslinking agents, processing aids (e.g., drainage aid), dispersing agent, flocculent, viscosity reducers, flame retardants, dispersing agents, plasticizers, antioxidants, compatibility agents, fillers, pigments, UV protectors, and the like. It is further contemplated that a foaming agent can be used to introduce a chemical bonding agent to a composite mycelium material. Such a foaming agent can make a web of composite mycelium material more porous by introducing air to the web.

### Plasticizers

Various plasticizers may be applied to the cultivated mycelium material or composite mycelium material to alter the mechanical properties of the cultivated mycelium material or composite mycelium material. In such embodiments, the cultivated mycelium material or composite mycelium material further includes a plasticizer. U.S. Pat. No. 9,555,395 discusses adding a variety of humectants and plasticization agents. Specifically, the U.S. Pat. No. 9,555,395 discusses using glycerol, sorbitol, triglyceride plasticizers, oils such as linseed oils, castor oils, drying oils, ionic and/or nonionic glycols, and polyethylene oxides. U.S. Patent Publication No. 2018/0282529 further discusses treating cultivated mycelium material or composite mycelium material with plasticizers such as glycerol, sorbitol or another humectant to retain moisture and otherwise enhance the mechanical properties of the cultivated mycelium material or composite mycelium material such as the elasticity and flexibility of the cultivated mycelium material or composite mycelium material. In such embodiments, the cultivated mycelium material or composite mycelium material is flexible.

Other similar plasticizers and humectants are well-known in the art, such as polyethylene glycol and fat liquors obtained by emulsifying natural oil with a liquid that is immiscible with oil (e.g. water) such that the micro-droplets of oil may penetrate the material. Various fat liquors contain emulsified oil in water with the addition of other compounds such as ionic and non-ionic emulsifying agents, surfactants, soap, and sulfate. Fat liquors may include various types of oil such as mineral, animal and plant-based oils.

### Tannins and Dyes

In various embodiments of the present disclosure, it may be ideal to impart color to the cultivated mycelium material or composite mycelium material. As discussed in U.S. Patent Publication No. 2018/0282529, tannins may be used to impart a color to cultivated mycelium material, composite mycelium material, or preserved composite mycelium material.

As cultivated mycelium material and/or composite mycelium material includes, in part, of chitin, it lacks the functional sites that are abundant in protein-based materials. Therefore, it may be necessary to functionalize the chitin in the cultivated mycelium material or composite mycelium material in order to create binding sites for acid and direct dyes. Methods of functionalizing chitin are discussed above.

Various dyes may be used to impart color to the cultivated mycelium material or composite mycelium material such as acid dyes, direct dyes, disperse dyes, sulfur dyes, synthetic dyes, reactive dyes, pigments (e.g. iron oxide black and cobalt blue) and natural dyes. In some embodiments, the cultivated mycelium material or composite mycelium material is submerged in an alkaline solution to facilitate dye uptake and penetration into the material prior to application of a dye solution. In some embodiments, the cultivated mycelium material or composite mycelium material is pre-soaked in ammonium chloride, ammonium hydroxide, and/or formic acid prior to application of a dye solution to facilitate dye uptake and penetration into the material. In some embodiments, tannins may be added to the dye solution. In various embodiments, the cultivated mycelium material or composite mycelium material may be preserved as discussed above before dye treatment or pre-treatment.

Depending on the embodiment, the dye solution may be applied to the cultivated mycelium material or composite mycelium material using different application techniques. In some embodiments, the dye solution may be applied to the one or more exterior surfaces of the cultivated mycelium material or composite mycelium material. In other embodiments, the cultivated mycelium material or composite mycelium material may be submerged in the dye solution.

In addition to pre-soaking with various solutions, agents may be added to the dye solution to facilitate dye uptake and penetration into the material. In some embodiments, ammonium hydroxide and/or formic acid with an acid or direct dye to facilitate dye uptake and penetration into the material. In some embodiments, an ethoxylated fatty amine is used to facilitate dye uptake and penetration into the processed material.

In various embodiments, a plasticization agent is added after or during the addition of the dye. In various embodiments, the plasticization agent may be added with the dye solution. In specific embodiments, the plasticization agent may be coconut oil, vegetable glycerol, or a sulfited or sulfated fat liquor.

In some embodiments, the dye solution may be maintained at a basic pH using a base such as ammonium hydroxide. In specific embodiments, the pH will be at least 9, 10, 11 or 12. In some embodiments, the pH of the dye solution will be adjusted to an acidic pH in order to fix the dye using various agents such as formic acid. In specific embodiments, the pH will be adjusted to a pH less than 6, 5, 4 or 3 in order to fix the dye.

In various methods, the cultivated mycelium material, composite mycelium material, and/or preserved composite mycelium material may be subject to mechanical working or agitation while the dye solution is being applied in order to facilitate dye uptake and penetration into the material. In some embodiments, subjecting the cultivated mycelium material, composite mycelium material, and/or preserved composite mycelium material to squeezing or other forms of pressure while in a dye solution enhanced dye uptake and penetration. In some embodiments, the cultivated mycelium material, composite mycelium material, and/or preserved composite mycelium material may be subject to sonication.

Using the methods described herein, the cultivated mycelium material or composite mycelium material may be dyed or colored such that the color of the processed cultivated mycelium material or composite mycelium material is substantially uniform. In some embodiments, the cultivated mycelium material or composite mycelium material is colored with the dye and the color of the cultivated mycelium material or composite mycelium material is substantially uniform on one or more surfaces of the cultivated mycelium material or composite mycelium material. Using the methods described above, the cultivated mycelium material or composite mycelium material may be dyed or colored such that dye and color is not just present in the surfaces of the cultivated mycelium material or composite mycelium material but instead penetrated through the surface to the inner core of the material. In such embodiments, the dye is present throughout the interior of the cultivated mycelium material or composite mycelium material.

In various embodiments of the present disclosure, the cultivated mycelium material or composite mycelium material may be dyed so that the cultivated mycelium material or composite mycelium material is colorfast. Colorfastness may be measured using various techniques such as ISO 11640:2012: Tests for Color Fastness—Tests for color fastness—Color fastness to cycles of to-and-fro rubbing or ISO 11640:2018 which is an update of ISO 11640:2012. In a specific embodiment, colorfastness will be measured according to the above using a Grey Scale Rating as a metric to determine rub fastness and change to sample. In some embodiments, the cultivated mycelium material or composite mycelium material will demonstrate strong colorfastness indicated by a Grey Scale Rating of at least 3, at least 4 or at least 5.

### Protein Sources

In various embodiments, it may be beneficial to optionally treat the cultivated mycelium material or composite mycelium material with one or more protein sources that are not naturally occurring in the cultivated mycelium material or composite mycelium material (i.e. exogenous protein sources). In some embodiments, the one or more proteins are from a species other than a fungal species from which the cultivated mycelium material is generated. In some embodiments, the cultivated mycelium material or composite mycelium material may be optionally treated with a plant protein source such as pea protein, rice protein, hemp protein and soy protein. In some embodiments, the protein source will be an animal protein such as an insect protein or a mammalian protein. In some embodiments, the protein will be a recombinant protein produced by a microorganism. In some embodiments, the protein will be a fibrous protein such as silk or collagen. In some embodiments, the protein will be an elastomeric protein such as elastin or resilin. In some embodiments, the protein will have one or more chitin-binding domains. Exemplary proteins with chitin-binding domains include resilin and various bacterial chitin-binding proteins. In some embodiments, the protein will be an engineered or fusion protein including one or more chitin-binding domains. Depending on the embodiment, the cultivated mycelium material or composite mycelium material may be preserved, as described above, before treatment or treated without prior preservation.

In a specific embodiment of the disclosure, the cultivated mycelium material or composite mycelium material is submerged in a solution including the protein source. In a specific embodiment, the solution including the protein source is aqueous. In other embodiments, the solution including the protein source includes a buffer such as a phosphate buffered saline.

In some embodiments, the solution including the protein source will include an agent that functions to crosslink the protein source. Depending on the embodiment, various known agents that interact with functional groups of amino acids can be used. In a specific embodiment, the agent that functions to crosslink the protein source is transglutaminase. Other suitable agents that crosslink amino acid functional groups include tyrosinases, genipin, sodium borate, and lactases. In other embodiments, traditional tanning agents may be used to crosslink proteins including chromium, vegetable tannins, tanning oils, epoxies, aldehydes and syntans. As discussed above, due to toxicity and environmental concerns with chromium, PAE other minerals may be used such as aluminum, titanium, zirconium, iron and combinations thereof with and without chromium.

In various embodiments, treatment with a protein source may occur before, after or concurrently with preserving the cultivated mycelium material or composite mycelium material, plasticizing the cultivated mycelium material or composite mycelium material and/or dyeing the cultivated mycelium material or composite mycelium material. In some embodiments, treatment with a protein source may occur before or during preservation of the cultivated mycelium material or composite mycelium material using a solution including alcohol and salt. In some embodiments, treatment with a protein source occurs before or concurrently with dyeing the cultivated mycelium material or composite mycelium material. In some of these embodiments, the protein source is dissolved in the dye solution. In a specific embodiment, the protein source will be dissolved in a basic dye solution optionally including one or more agents to facilitate dye uptake.

In some embodiments, a plasticizer will be added to the dye solution including the dissolved protein source to concurrently plasticize the cultivated mycelium material or composite mycelium material. In a specific embodiment, the plasticizer may be a fat liquor. In a specific embodiment, a plasticizer will be added to a protein source that is dissolved in a basic dye solution including one or more agents to facilitate dye uptake.

### Coating and Finishing Agents

After a cultivated mycelium material or composite mycelium material has been processed using any combination of methods as described above, the cultivated mycelium material or composite mycelium material may be treated with a finishing agent or coating. Various finishing agents common to the leather industry such as proteins in binder solutions, nitrocellulose, synthetic waxes, natural waxes, waxes with protein dispersions, oils, polyurethane, acrylic polymers, acrylic resins, emulsion polymers, water-resistant polymers and various combinations thereof may be used. In a specific embodiment, a finishing agent including nitrocellulose may be applied to the cultivated mycelium material or composite mycelium material. In another specific embodiment, a finishing agent including conventional polyurethane finish will be applied to the cultivated mycelium material or composite mycelium material. In various embodiments, one or more finishing agents will be applied to the cultivated mycelium material or composite mycelium material sequentially. In some instances, the finishing agents will be combined with a dye or pigment. In some instances, the finishing agents will be combined with a handle modifier (i.e. feel modifier or touch) including one or more of natural and synthetic waxes, silicone, paraffins, saponified fatty substances, amides of fatty acids, amides esters, stearic amides, emulsions thereof, and any combination of the foregoing. In some instances, the finishing agents will be combined with an antifoam agent. In some embodiments, an external force is applied to the cultivated mycelium material or composite mycelium material. In such embodiments, the external force includes heating and/or pressing.

### Processed Mycelium Material

In various embodiments of the present disclosure, the cultivated mycelium material or composite mycelium material may be mechanically processed and/or chemically processed in different ways both in solution (i.e. dye solution, protein solution or plasticizer) and after the cultivated mycelium material or composite mycelium material has been removed from the solution. In such embodiments, the method includes mechanically processing and/or chemically processing the cultivated mycelium material or composite mycelium material, wherein a processed mycelium material is produced.

While the cultivated mycelium material or composite mycelium material is in a solution it may be agitated, sonicated, squeezed or pressed to ensure uptake of the solution. The degree of mechanical processing will depend on the specific treatment being applied and the level of fragility of the cultivated mycelium material or composite mycelium material at its stage in processing. Squeezing or pressing of the cultivated mycelium material or composite mycelium material may be accomplished by hand wringing, mechanical wringing, a platen press, a lino roller or a calendar roller.

Similarly, as discussed above, the cultivated mycelium material or composite mycelium material may be pressed or otherwise worked to remove solution from the composite mycelium material after it is removed from solution. Treating with a solution and pressing the material may be repeated several times. In some embodiments, the material is pressed at least two times, at least three times, at least four times, or at least five times.

Once the cultivated mycelium material or composite mycelium material is fully dried (e.g. using heat, pressing or other desiccation techniques described above), the cultivated mycelium material or composite mycelium material may be subject to additional mechanical- and/or chemical-processing. Depending on the technique used to treat the cultivated mycelium material or composite mycelium material and the resultant toughness of the cultivated mycelium material or composite mycelium material, different types of mechanical processing may be applied including but not limited to sanding, brushing, plating, staking, tumbling, vibration and cross-rolling.

In some embodiments, the cultivated mycelium material or composite mycelium material may be embossed with any heat source or through the application of chemicals. In some embodiments, the cultivated mycelium material or composite mycelium material in solution may be subjected to additional chemical processing, such as, e.g., being maintained at a basic pH using a base such as ammonium hydroxide. In specific embodiments, the pH will be at least 9, 10, 11 or 12. In some embodiments, the pH of the cultivated mycelium material or composite mycelium material in solution will be adjusted to an acidic pH in order to fix the composite mycelium material using various agents such as formic acid. In specific embodiments, the pH will be adjusted to a pH less than 6, 5, 4 or 3 in order to fix the cultivated mycelium material or composite mycelium material.

Finishing, coating and other steps may be performed after or before mechanical processing and/or chemical processing of the dried cultivated mycelium material or composite mycelium material. Similarly, final pressing steps, including ornamental steps such as embossing or engraving, may be performed after or before mechanical processing and/or chemical processing of the dried cultivated mycelium material or composite mycelium material.

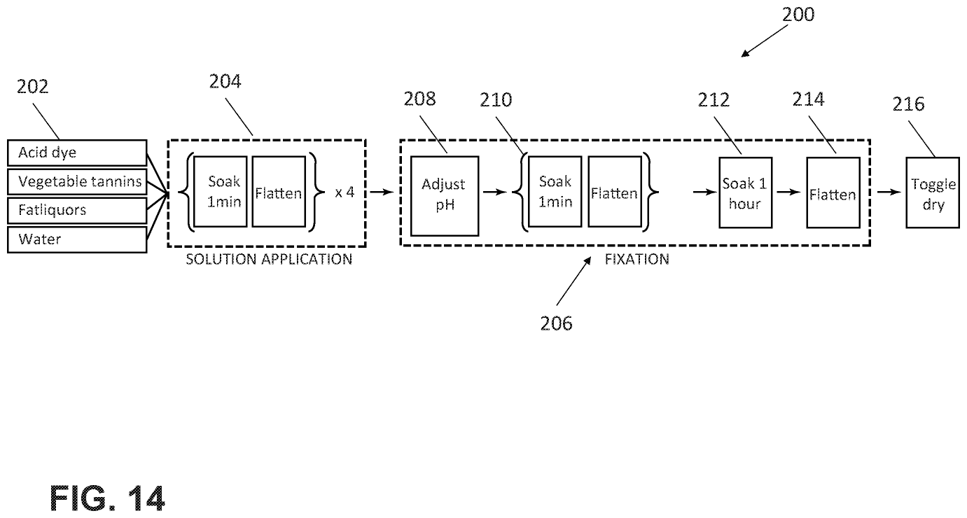

FIG. 14 illustrates a flow chart of a method 200 for converting raw mycelium material into a crust material that can be treated according to a desired finishing process (e.g., finishing coatings, ornamental steps, final pressing steps) based on the end-use application of the material. The raw mycelium material can be dried, refrigerated, or frozen material made according to any of the processes described herein. The raw material may optionally be split on the top and/or bottom to provide a mycelium panel having the desired thickness. Splitting can also provide a smoother surface at the cut. The crust material can be dyed, plasticized, dried and/or otherwise post-processed as described herein.

Still referring to FIG. 14, at step 202 a pre-finishing treatment solution can be prepared based on the dimensions and mass of the mycelium material. In one example, the pre-finishing treatment solution can be prepared at a volume of about 6 mL per gram of wet mycelium material or 20 mL per gram of dried mycelium material. The pre-finishing treatment solution can include one or more dyes, tannins, and/or plasticizers (e.g. fat liquors) in a suitable solvent, such as water. In one example, the pre-finishing treatment solution includes one or more dyes and/or tannins and one or more fat liquors. The amount of dye added can be based on the particular type of dye and the desired color of the resulting product. An exemplary pre-finishing treatment solution includes: one or more acid dyes at a concentration to produce the desired color; about 25 g/L vegetable tannins; about 6.25 g/L Truposol® LEX fatliquour (Trumpler, Germany); and about 18 g/L to about 19 g/L Trupon® DXV fatliquor (Trumpler, Germany).

At step 204, the pre-finishing treatment solution can be applied to the mycelium material through a combination of soaking and flattening processes. In one example, the material is soaked in the pre-finishing treatment solution for a predetermined period of time (e.g., 1 minute) and then moved through a flattening system. An example of a suitable flattening system includes moving the soaked material through a pair of rollers that are spaced to provide the desired degree of flattening to the material with each pass between the rollers. The material can be pushed and/or pulled through the rollers. The rate at which the material is passed through the rollers can vary. According to one aspect of the present disclosure, the soaking and flattening process at step 204 can be repeated one or more times (e.g., 1, 2, 3, 4, 5 or more times).

Following the pre-finishing treatment application at 204, the material can proceed to a fixation process 206. The fixation process 206 includes adjusting the pH of the pre-finishing treatment solution to a pH suitable for fixing the dyes. In one example, the fixation process is an acid fixing process that includes decreasing the pH of the pre-finishing treatment solution. Non-limiting examples of acids suitable for acid fixing include acetic acid and formic acid. For example, acetic acid can be used to decrease the pH of the exemplary pre-finishing treatment solution described above to a pH of 3.15±10.

At step 210, the mycelium material can be soaked in the pH adjusted pre-finishing treatment solution and flattened in a manner similar to that described above with regard to step 204. The soaking and flattening process at step 210 can be repeated one or more times (e.g., 1, 2, 3, 4, 5 or more times).

Step 212 includes a final, extended soak of the material in the pH adjusted pre-finishing treatment solution. The material can be inverted about half way through the extended soak period. The extended soak period can be from about 30 minutes to 1 hour or more. When the extended soak time period is complete, at 214 the material can be processed through a final flattening process. The final flattening process can be the same or different than that described above with regard to steps 204 and 210.

Following the fixation process 206, at step 216 the material can be dried with or without heating. The material can be held generally vertically, horizontally, or any orientation therebetween during the drying step 216. The material may optionally be restrained during the drying step. For example, one or more clamps may be used to restrain all or a portion of the material during drying. In some examples, the drying step 216 is conducted at ambient conditions.

### Mechanical Properties of Mycelium Material

Various methods of the present disclosure may be combined to provide processed cultivated or composite mycelium material that has a variety of mechanical properties. In such embodiments, the mycelium material includes a mechanical property, e.g., a wet tensile strength, an initial modulus, an elongation percentage at the break, a thickness, and/or a slit tear strength. Other mechanical properties include, but are not limited to, elasticity, stiffness, yield strength, ultimate tensile strength, ductility, hardness, toughness, creep resistance, and other mechanical properties known in the art.

In various embodiments, the processed mycelium material may have a thickness that is less than 1 inch, less than ½ inch, less than ¼ inch or less than ⅛ inch. In some embodiments, the composite mycelium material has a thickness of about 0.5 mm to about 3.5 mm, inclusive, e.g., about 0.5 mm to about 1.5 mm, about 1 mm to about 2.5 mm, and about 1.5 mm to about 3.5 mm. The thickness of the material within a given piece of material may have varying coefficients of variance. In some embodiments, the thickness is substantially uniform to produce a minimal coefficient of variance.

In some embodiments, the mycelium material can have an initial modulus of at least 20 MPa, at least 25 MPa, at least 30 MPa, at least 40 MPa, at least 50 MPa, at least 60 MPa, at least 70 MPa, at least 80 MPa, at least 90 MPa, at least 100 MPa, at least 110 MPa, at least 120 MPa, at least 150 MPa, at least 175 MPa, at least 200 MPa, at least 225 MPa, at least 250 MPa, at least 275 MPa, or at least 300 MPa. In some embodiments, the mycelium material may have an initial modulus of about 0.5 MPa to about 300 MPa, inclusive, for example about 0.5 MPa to about 10 MPa, about 1 MPa to about 20 MPa, about 10 MPa to about 30 MPa, about 20 MPa to about 40 MPa, about 30 MPa to about 50 MPa, about 40 MPa to about 60 MPa, about 50 MPa to about 70 MPa, about 60 MPa to about 80 MPa, about 70 MPa to about 90 MPa, about 80 MPa to about 100 MPa, about 90 MPa to about 150 MPa, about 100 MPa to about 200 MPa, and about 150 MPa to about 300 MPa. In specific embodiments, the mycelium material has an initial modulus of 0.8 MPa. In one aspect, the mycelium material has an initial modulus of 1.6 MPa. In another aspect, the mycelium material has an initial modulus of 97 MPa.

In some embodiments, the mycelium material can have a wet tensile strength of about 0.05 MPa to about 50 MPa, inclusive, e.g., about 1 MPa to about 5 MPa, about 5 MPa to about 20 MPa, about 10 MPa to about 30 MPa, about 15 MPa to about 40 MPa, and about 20 MPa to about 50 MPa. In specific embodiments, the mycelium material may have a wet tensile strength of about 5 MPa to about 20 MPa. In one aspect, the mycelium material has a wet tensile strength of about 7 MPa. In a specific embodiment, the wet tensile strength will be measured by ASTM D638.

In some embodiments, the mycelium material can have a breaking strength (“ultimate tensile strength”) of at least 1.1 MPa, at least 6.25 MPa, at least 10 MPa, at least 12 MPa, at least 15 MPa, at least 20 MPa, at least 25 MPa, at least 30 MPa, at least 35 MPa, at least 40 MPa, at least 45 MPa, at least 50 MPa.

In some embodiments, the mycelium material has an elongation at the break of less than 2%, less than 3%, less than 5%, less than 20%, less than 25%, less than 50%, less than 77.6%, or less than 200%. For example, the mycelium material may have an elongation at the break of about 1% to about 200%, inclusive, e.g., about 1% to about 25%, about 10% to about 50%, about 20% to about 75%, about 30% to about 100%, about 40% to about 125%, about 50% to about 150%, about 60% to about 175%, and about 70% to about 200%.

In some embodiments, the initial modulus, ultimate tensile strength, and elongation at the break are measured using ASTM D2209 or ASTM D638. In a specific embodiment, the initial modulus, ultimate tensile strength, and elongation at the break are measured using a modified version ASTM D638 that uses the same sample dimension as ASTM D638 with the strain rate of ASTM D2209.

In some embodiments, the mycelium material can have a single stitch tear strength of at least 15N, at least 20N, at least 25N, at least 30N, at least 35N, at least 40N, at least 50N, at least 60N, at least 70N, at least 80N, at least 90N, at least 100N, at least 125N, at least 150N, at least 175N, or at least 200N. In a specific embodiment, the tongue tear strength will be measured by ASTM D4786.

In some embodiments, the mycelium material can have a double stitch tear strength of at least 20N, at least 40N, at least 60N, at least 80N, at least 100N, at least 120N, at least 140N, at least 160N, at least 180N, or at least 200N. In a specific embodiment, the tongue tear strength will be measured by ASTM D4705.

In some embodiments, the mycelium material can have a tongue tear strength (also referred to as slit tear strength) of at least 1.8N, at least 15N, at least 25N, at least 35N, at least 50N, at least 75N, at least 100N, at least 150N, or at least 200N, as measured by ISO-3377. In some embodiments, the mycelium material may have a slit tear strength of at least 1N, at least 20N, at least 40N, at least 60N, at least 80N, at least 100N, at least 120N, at least 140N, at least 160N, at least 180N, or at least 200N, as measured by ISO-3377-2. In one aspect, the mycelium material has a slit tear strength of about 1N to about 200N, inclusive, e.g., about 10N to about 30N, about 20N to about 40N, about 30N to about 50N, about 40N to about 60N, about 50N to about 70N, about 60N to about 80N, about 70N to about 90N, about 80N to about 100N, about 90N to about 110N, about 100N to about 120N, about 110N to about 130N, about 120N to about 140N, about 130N to about 150N, about 140N to about 160N, about 150N to about 170N, about 160N to about 180N, about 170N to about 190N, and about 180N to about 200N, as measured by ISO-3377-2.

In some embodiments, the mycelium material can have a flexural modulus (Flexure) of at least 0.2 MPa, at least 1 MPa, at least 5 MPa, at least 20 MPa, at least 30 MPa, at least 50 MPa, at least 80 MPa, at least 100 MPa, at least 120 MPa, at least 140 MPa, at least 160 MPa, at least 200 MPa, at least 250 MPa, at least 300 MPa, at least 350 MPa, at least 380 MPa. In a specific embodiment, the compression will be measured by ASTM D695.

In various embodiments of the present disclosure, the mycelium material has different absorption properties measured as a percentage mass increase after soaking in water. In some embodiments, the percent mass increase after soaking in water for 1 hour is less than 1%, less than 5%, less than 25%, less than 50%, less than 74%, or less than 92%. In a specific embodiment, the percent mass increase after soaking in water after 1 hour is measured using ASTM D6015.

### Methods of Producing a Mycelium Material

Also provided is a method of producing a mycelium material as described herein. According to one embodiment of the disclosure, a mycelium material can be produced by generating a cultivated mycelium material including one or more masses of branching hyphae; disrupting the cultivated mycelium material including the one or more masses of branching hyphae; and adding a bonding agent to the cultivated mycelium material; thus producing the composite mycelium material. In some embodiments, the cultivated mycelium material includes one or more masses of disrupted branching hyphae. In some embodiments, the one or more masses of disrupted branching hyphae has a length. In such embodiments, the one or more masses of disrupted branching hyphae has a length of about 0.1 mm to about 5 mm.

In some embodiments, the generating comprises generating cultivated mycelium material on a solid substrate. In some embodiments, the method further comprises incorporating a supporting material into the mycelium material. In some embodiments, the supporting material is a reinforcing material. In some embodiments, the supporting material is a base material. In some embodiments, the disrupting comprises disrupting the one or more masses of branching hyphae by a mechanical action. In some embodiments, the method further comprises adding one or more proteins that are from a species other than a fungal species from which the cultivated mycelium material is generated. In some embodiments, the method further comprises adding a dye to the cultivated mycelium material or the mycelium material. In some embodiments, the method further comprises adding a plasticizer to the cultivated mycelium material or the mycelium material. In some embodiments, the method further comprises adding a tannin to the cultivated mycelium material or the mycelium material. In some embodiments, the method further comprises adding a finishing agent to the mycelium material. In some embodiments, the method further comprises determining a mechanical property of the mycelium material, wherein the mechanical property includes, but is not limited to, wet tensile strength, initial modulus, elongation percentage at the break, thickness, slit tear strength, elasticity, stiffness, yield strength, ultimate tensile strength, ductility, hardness, toughness, creep resistance, and the like. For example, the mycelium material has a wet tensile strength of about 0.05 MPa to about 50 MPa, an initial modulus of about 0.5 MPa to about 300 MPa, an elongation percentage at the break of about 1% to about 200%, a thickness of about 0.5 mm to about 3.5 mm, and/or a slit tear strength of about 1 N to about 200 N.

In some embodiments, the cultivated mycelium material or composite mycelium material is produced using traditional paper milling equipment.

### EXAMPLES

The following examples are put forth so as to provide those of ordinary skill in the art with a complete disclosure and description of how to make and use aspects of the present disclosure, and are not intended to limit the scope of what is regarded as the scope of the present disclosure, nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., g, grams; RT, room temperature (about 25° C.);”, inch; mL, milliliter; mm, millimeter; mM, millimolar; L, liter; rpm, revolutions per minute; bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Materials and Methods

The following material and methods were used in the Examples.

### Mycelium Material Samples

For each of the samples (A)-(BB) described below and in Examples 1-4, components were blended together in a blender (Vitamix or Blendtec). The resulting slurry was poured into a mold resting on a paper-making screen or forming cloth that let water pass through. After waiting approximately 1-15 minutes, the mold was removed from the slurry. The material was then pressed via a hand press to about 0.25 inches. The resulting material was then removed from the screen and dried in front of a fan. The sample was dried and then pressed in a heated press. A scaffold was optionally included in the mycelium material as described below.

For each of the samples discussed below in Examples 5 to 7, Ganoderma sessile, Phycomyces blakesleeanus, or Neurospora crassa was cultivated to form a substantially homogenous (i.e., devoid of any fruiting bodies or substantial morphological variations) mass of mycelium material. The cultivated mycelium material was grown by first inoculating a solid or liquid substrate with an inoculum of the mycelium from the selected species of fungus.

The following samples (A)-(BB) were used:

(A) HM1-1-1: 15 g dry cultivated mycelium material, 375 mL water, and 3 g pea protein (Nutribiotic) were blended together. 3.75 g BDF TG was added. The blend was mixed with a spatula and incubated for 30 min at room temperature (RT), and then poured into a 6×6 inch mold, pressed to ¼″ thick, dried, and labeled HM1-1-1. A third of this material was rubbed with 3 g of epoxidized soybean oil and the sample was then pressed at 120° C. for 1 min at 1 metric ton of pressure, and labeled HM1-1-11_120 p.

(B) HM1-1-7: 15 g dry cultivated mycelium material, 375 mL water, 3 g pea protein (Nutribiotic), and 3 g leather glue were blended together. 3.75 g BDF TG was added. The blend was mixed with a spatula and incubated for 30 min at RT, and then poured into a 6×6 inch mold, pressed to ¼″ thick, dried, and labeled HM1-1-7.

(C) HM1-1-9: 2.5 g dry cultivated mycelium material, 75 mL water, and 0.5 g pea protein (Nutribiotic) were blended together, and poured into a 6×6 inch mold, pressed to ¼″ thick, dried, and labeled HM1-1-9.

(D) HM1-1-11: 10 g dry cultivated mycelium material, 400 mL water, 4 g pea protein (Nutribiotic), and 7.5 g epoxidized soybean oil were blended together, and poured into a 6×6 inch mold, pressed to ¼″ thick, dried, and labeled HM1-1-11. Half the sample was then pressed at 120° C. for 1 min at 1 metric ton of pressure.

(E) HM0 referred to a blended sample made with 15 g dry cultivated mycelium material, 3 g pea protein and 5% glycerol in 400 mL of water.

(F) HM25: 5 g dry cultivated mycelium material, 125 mL water, 125 mL of 1.5% PAE resin (Polycup 9200 from Solenis) in 40 mM phosphate buffer at pH=7, and 1 g pea protein were blended together. Two 2×2 inch squares were made. One was heated for 5 minutes at 105° C. (Labeled: HM25_5 min) and one was heated for 10 minutes at 105° C. (Labeled: HM25_10).

(G) HM1-3-1: 5 g cultivated mycelium material, 125 mL of 1.5% PAE in 50 mM phosphate buffer (pH=7.4), and 1 g pea protein were blended together. Two 2×2 inch mats were made. The mats were heated at 105° C. for 5 min; it took 5 min for the oven to reach 105° C. after putting the mats in. Then, one mat was soaked in 5% glycerol for 10 minutes and dried in the fume hood, the other was wet tensile tested as is.

(H) HM1-3-2: 5 g cultivated mycelium material, 125 mL of 1.5% PAE in 50 mM phosphate buffer (pH=7.4), 2.5 g leather glue from Eco-Flo®, and 1 g pea protein were blended together. Two 2×2 inch mats were made and they were heated at 105° C. for 5 min; it took 5 min for the oven to reach 105° C. after putting the mats in. Then, one mat was soaked in 5% glycerol for 10 minutes and dried in the fume hood, the other was wet tensile tested as is.

(I) HM1-3-3: 15 g cultivated mycelium material, 400 mL of 1.5% PAE in 50 mM phosphate buffer (pH=7.4), and 3 g pea protein were blended together. After drying, salt crystals formed on the outside of the homogenized mycelia panel. One 6×6 inch mat was made and heated at 105° C. for 5 min; it took 5 min for the oven to reach 105° C. after putting the mats in. Then, the mat was soaked in 5% glycerol for 10 minutes and dried in the fume hood.

(J) HM1-3-4: 5 g cultivated mycelium material, 125 mL of 1.5% PAE in 25 mM phosphate buffer (pH=7.4), and 1 g pea protein were blended together. Two 2×2 inch mats were made; the mats were poured into a 2×2 inch mold and then rolled with a baking pin unidirectionally between two papermaking screens. The orientation with the pin was parallel to the longer side of the rectangular panels.

(K) HM1-3-5: 5 g cultivated mycelium material, 125 mL of 3.0% PAE in 25 mM phosphate buffer (pH=7.4), and 1 g pea protein were blended together. Two 2×2 inch mats were made. These mats were heated at 105° C. for 5 min; it took 5 min for the oven to reach 105° C. after putting the mats in.

(L) HM1-3-6: 5 g cultivated mycelium material, 125 mL of 1.5% PAE in 25 mM phosphate buffer (pH=7.4), 2.5 g leather glue from Eco-Flo®, and 1 g pea protein were blended together. A cotton textile scaffold (scaffold2) was incorporated in the center of two 2×2 inch mats. One panel was pressed to 1 metric ton at 105° C. for 2 min, the other panel was heated at 105° C. for 5 min, after waiting 5 minutes for the oven to reach 105° C. A 1.54 mm spacer was used to limit the degree to which the panel was pressed.

(M) HM1-3-7: 5 g cultivated mycelium material, 125 mL of 1.5% PAE in 25 mM phosphate buffer (pH=7.4), 5 g leather glue from Eco-Flo®, and 1 g pea protein were blended together. A cotton textile scaffold (scaffold2) was incorporated in the center of two 2×2 inch mats. One panel was pressed to 1 metric ton at 105° C. for 2 min, the other panel was heated at 105° C. for 5 min, after waiting 5 minutes for the oven to reach 105° C. A 1.54 mm spacer was used to limit the degree to which the panel was pressed.

(N) HM1-3-8: 5 g cultivated mycelium material and 125 mL of 1.5% PAE in 25 mM phosphate buffer (pH=7.4) were blended together. Two 2×2 inch mats were made. These mats were heated at 105° C. for 5 min; it took 5 min for the oven to reach 105° C. after putting the mats in.

(O) HM1-3-9: 5 g cultivated mycelium material, 125 mL of 1.5% PAE in 25 mM phosphate buffer (pH=7.4), and 1 g pea protein were blended together. Two 2×2 inch mats were made. These mats were pressed at 1 metric ton at 105° C. for 2 minutes to a height of 1.45 mm.

(P) HM1-3-10: 5 g cultivated mycelium material, 125 mL of 1.5% PAE in 25 mM phosphate buffer (pH=7.4), and 1 g pea protein were blended together. A textile scaffold (scaffold2) was incorporated into one 2×2 inch mat. The scaffold was coated in dried cultivated mycelium material that had been poured over the scaffold in a dilute slurry the day before and allowed to dry. The panel was then pressed to 1.5 mm at 105° C. for 2 min at 1 metric ton of pressure.

(Q) HM1-3-11: 5 g cultivated mycelium material, 125 mL of 1.5% PAE in 25 mM phosphate buffer (pH=7.4), and 1 g pea protein were blended together. Two 2×2 inch mats were made. The mats and a cotton textile scaffold (scaffold4) with ⅛ inch pores were coated with Weldwood® contact cement and pressed at room temperature with 2 L of water in a beaker for 2.5 h. Then, the material was pressed to 2.54 mm for 4 min at 105° C. to 1 metric ton of pressure.

(R) HM1-3-12: 2.5 g cultivated mycelium material, 62.5 mL of 1.5% PAE in 25 mM phosphate buffer (pH=7.4), and 0.5 g pea protein were blended together. A papermaking scaffold (Scaffold3, black, non-textile, plastic) was incorporated into one 2×2 inch mat. The panel was then pressed to 1.5 mm at 105° C. for 2 min at 1 metric ton of pressure.

(S) HM1-3-13: 2.5 g cultivated mycelium material, 62.5 mL of 1.5% PAE in 25 mM phosphate buffer (pH=7.4), and 0.5 g pea protein were blended together. One 2×2 inch mat was made with a scaffold 4 incorporated inside that had had mycelia slurry poured over it the night before. The panel was then pressed to 1.5 mm at 105° C. for 2 min at 1 metric ton of pressure.

(T) HM1-3-14: 2.5 g cultivated mycelium material, 62.5 mL of 1.5% PAE in 25 mM phosphate buffer (pH=7.4), and 0.5 g pea protein were blended together. One 2×2 inch mat with a clean scaffold 4 was incorporated inside. The panel was then pressed to 1.5 mm at 105° C. for 2 min at 1 metric ton of pressure.

(U) HM1-3-15: 5 g cultivated mycelium material, 125 mL of 1.5% PAE in 25 mM phosphate buffer (pH=7.4), and 1 g pea protein were blended together. Two 2×2 inch mats were made. The mats and a cotton textile scaffold (scaffold4) with ⅛ inch pores were coated with leather tack glue from Springfield Leather Company and pressed at room temperature with 2 L of water in a beaker for 2.5 h. Then, the material was pressed to 2.54 mm for 4 min at 105° C. to 1 metric ton of pressure.

(V) HM1-4-1: 5 g cultivated mycelium material, 125 mL of 1.5% PAE in 25 mM phosphate buffer (pH=7.4), 1 g pea protein, 1 g iron (Ill) oxide black or 1 g of cobalt blue, and 5% glycerol were blended together. A cotton textile scaffold (scaffold4) was incorporated inside. Two 2×2 inch mats were made. These mats were pressed and heated at 105° C. for 2 min at 1 metric ton of pressure.

(W) HM1-4-2: 5 g cultivated mycelium material, 125 mL of 1.5% PAE in 25 mM phosphate buffer (pH=7.4), 1 g pea protein, 0.125 g brown acid dye, and 5% glycerol were blended together. A cotton textile scaffold (scaffold4) was incorporated inside. Two 2×2 inch mats were made. These mats were pressed and heated at 105° C. for 2 min at 1 metric ton of pressure.

(X) HM1-4-3: 15 g cultivated mycelium material, 400 mL of 1.5% PAE in 25 mM phosphate buffer (pH=7.4), 3 g pea protein, 5 g iron (Ill) oxide black, and 5% glycerol were blended together. Two 6×6 inch mats were made. These mats were pressed and heated at 105° C. for 2 min at 1 metric ton of pressure.

(Y) HM1-4-4: Same as HM1-4-3.

(Z) HM1-4-5: Same as HM1-4-3 and HM1-4-4, except that 8 g of leather glue from Eco-Flo® was also blended together.

(AA) HM3:15 g dry cultivated mycelium material, 500-600 mL water, and 3 g pea protein (Nutribiotic) were blended together. 3.75 g BDF TG was added, mixed with a spatula, poured half the mold into a 6×6 inch mold, pressed a pre-wetted scaffold1 into the material, and poured the other half of the material into the mold. The mixture was incubated for 30 min, then pressed to ¼″ thick, and dried. This sample was cut in half, and 3 g of epoxidized soybean oil was rubbed into half the sample. The sample was then heat pressed at 1 metric ton of pressure at 120° C. for 2 min.

(BB) HM22: 15 g dry cultivated mycelium material, 550 mL water, and 3 g pea protein were blended together. One cheesecloth (scaffold1) was incorporated inside through crochet needling. Scaffold1 was not evenly placed in the middle of the material.

### Wet Tensile Testing

The standard test method for tensile testing of mycelium materials was performed according to the ASTM D638 protocol. Samples were conditioned at 65±2% RH for 24 hours. In some embodiments, samples were soaked in water for 1 hour at room temperature prior to testing. ASTM standard dies such as an ASTM D638 type IV dogbone was used to punch out samples. Each sample's thickness, width, and mass were measured. The appropriate tensile test method was then run on a universal testing machine from Zwick (zwikiLine Materials Testing Machine Z5.0 TH).

### Slit Tear Testing

The standard test method for slit tear testing of mycelium materials was performed according to the ISO 3377-2 protocol, using the universal testing system from Zwick. Samples were conditioned at 65±2% RH for 24 h. In some embodiments, samples were equilibrated at 65% relative humidity for 16 h at room temperature prior to testing. The ISO 3377-2 die was used to cut out 1″×2″ specimens with a center slit. Each specimen's thickness and mass were measured. The appropriate slit tear test method was then run on the universal mechanical tester from Zwick.

### Drafting of Mycelium Materials

The mycelium hyphae were aligned by manually drafting a thin sheet of material along direction. The drafting force applied to the material did not exceed the breaking force.

### Scanning Electron Microscopy (SEM) Imaging and Fourier Transform (FT) Analysis

Scanning electron microscopy (SEM) used a focus electron beam to assess the morphology of materials through the secondary electrons. The electron beam was scanned in a raster pattern to collect micrographs at scales between 1 mm and 10 nm or between 10× and 100,000× magnification. The SEM method used low vacuum (1 to 10 torr), avoiding the need for dehydrating or sputter coating biological samples.

SEM micrographs were then cropped to a square size and analyzed using Fourier transform (FT). The FT of an image represented a sum of complex exponentials of varying magnitudes (i.e. intensity), frequencies, and phase angle. The resulting frequency domain revealed the periodicity in the image as a function of the angle. Because aligned fibers gave rise to a periodicity orthogonal to the fiber axis, the frequency domain was used to quantity the preferential fiber alignment. The polar coordinate frequency domain image was then transformed into Cartesian coordinates to extract the profile of the azimuthal distribution. The azimuthal distribution was then fitted with a Gaussian peak to calculate the full-width at half-maximum and the maximum angular position.

### Polarized Fourier Transform Infrared (FTIR) Spectroscopy

Fourier Transform Infrared (FTIR) spectra were used to assess the secondary and tertiary structures of mycelium materials. Depending on the embodiment, FTIR spectra at different wavenumbers (cm⁻¹) may be used to assess the different chemical functions present in the chitin of mycelium hypha. The wavenumbers corresponding to the methyl deformation mode of the N-acetyl group was found to be about 1410 cm⁻¹ while the ether vibration mode was found to be about 950 cm⁻¹.

In attenuated total reflection mode, the infrared light beam was internally reflected inside the internal reflection element. The light absorbance arose from the attenuation of the evanescent wave at the interface.

In polarized FTIR, the light was polarized along with the s (perpendicular to the reflection plane). The sample was either angularly positioned along the polarized light s vector (0 degrees) or perpendicular (90 degrees). The ratio of the absorbance at 0 and 90 degrees defined the dichroic ratio (R) from which the second Legendre order parameter could be calculated <P2>=(R−1)/(R−2).

### Example 1: Tensile Properties of Mycelium Materials

As shown in the following results, by incorporating a bonding agent such as the crosslinker polyamide-epichlorohydrin (PAE) and optionally a textile scaffold, the wet tensile strength and the tear strength of the material improved dramatically. PAE is a crosslinker that is traditionally used in the paper industry, but is also found in sausage casings. Stiffer textile scaffolds performed better in the mycelium materials than less-stiff, more stretchy scaffolds. Without intending to be bound by any particular theory, it is proposed that less-stiff scaffolds did not end up bearing any load when incorporated into the materials. Also, less-stiff scaffolds were more likely to delaminate upon breaking than stiffer scaffolds. Water-based latex adhesives provided further benefits for the material in terms of both strength and plastic deformation.

In some samples including PAE, the composite mycelium materials had a higher wet tensile strength and a higher slit tear than intact cultivated mycelium material. In some samples having PAE, the composite mycelium materials had a lower elongation at the break (plastic deformation) than intact cultivated mycelium material.

Adding a Bonding Agent

Table 1 depicts wet tensile strength (MPa), initial modulus (MPa), and elongation at the break (%) of various mycelium materials.

TABLE 1

| | | Wet Tensile | | Initial Modulus | | Elongation at | |
|---|---|---|---|---|---|---|---|
| | | strength (MPa) | | (MPa) | | the break (%) | |
| | | | Std. | | Std. | | Std. |
| n | Sample | Average | Dev. | Average | Dev | Average | Dev |
| 3 | No PAE control | 0.11 | 0.00 | 1.97 | 0.78 | 8.20 | 0.11 |
| | (HM1-1-9) | | | | | | |
| 5 | PAE (HM1-3-8) | 0.86 | 0.17 | 3.48 | 0.74 | 14.46 | 1.81 |
| 3 | PAE, pea protein | 0.99 | 0.05 | 5.56 | 0.50 | 14.37 | 1.69 |
| | (HM25_5min) | | | | | | |
| 5 | PAE, pea protein, | 1.18 | 0.22 | 5.50 | 0.35 | 20.53 | 2.33 |
| | latex (HM1-3-2) | | | | | | |
| 5 | PAE, pressed | 1.80 | 0.22 | 15.26 | 3.21 | 12.70 | 1.66 |
| | (HM1-3-9p) | | | | | | |
| 4 | PAE, Scaffold3, pressed | 7.38 | 1.71 | 96.98 | 14.53 | 10.39 | 3.45 |
| | (HM1-3-12) | | | | | | |
| 4 | PAE, Scaffold4, pressed | 2.15 | 0.57 | 13.38 | 1.30 | 17.77 | 12.28 |
| | (HM1-3-13) | | | | | | |
| 4 | PAE, Scaffold4, glue, pressed | 5.91 | 1.48 | 39.68 | 3.53 | 20.20 | 1.85 |
| | (HM1-3-15) | | | | | | |
| 6 | Drafted mycelium polyurethane | 4.11 | 0.45 | 59.72 | 11.59 | 278.5 | 407.7 |
| | (PU) composite | | | | | | |

Adding a bonding agent such as PAE or polyurethane and in some samples, a textile scaffold, the tensile properties of the composite mycelium materials improved dramatically. The wet tensile strength increased from 0.11 MPa to at least 0.86 MPa, and up to 7.38 MPa. The initial modulus increased from 1.97 MPa to at least 3.48 MPa, and up to 96.98 MPa. The elongation at the break increased from 8.20% to at least 10.39%, and up to 278.5%. Thickness of the composite mycelium material ranged from 0.5 mm to 3.5 mm. Sub-network size, e.g., length, of the disrupted mycelium material ranged from 0.5 mm to 2 mm.

PAE Crosslinking

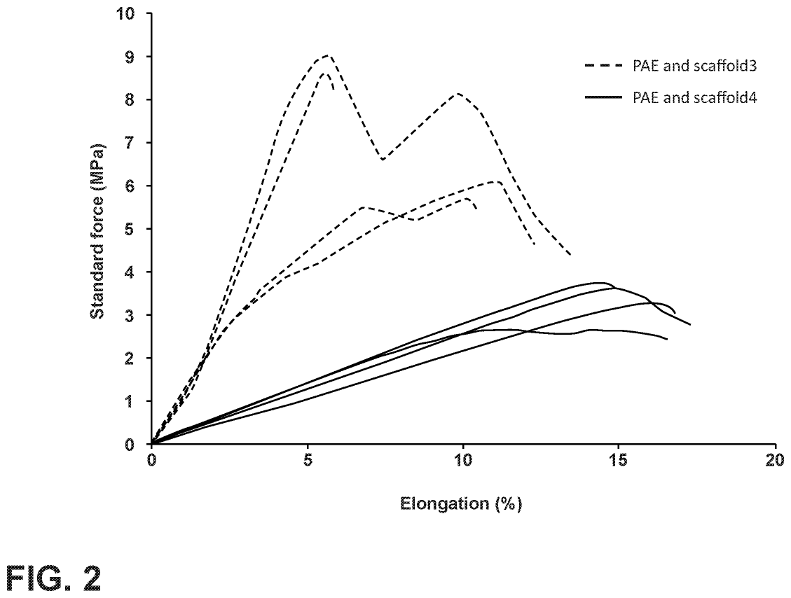

The presence of pea protein did not impact the PAE crosslinking. Sample HM1-3-8 was crosslinked with 1.5% PAE and no pea protein (Table 2). Increasing the concentration of PAE from 1.5% to 3% did not increase the wet tensile strength significantly (Table 2). FIG. 2 illustrates the stress-strain curves of a mycelia panel containing PAE and Scaffold3 that was heat-pressed (HM1-3-12) (dashed lines), and a mycelia panel containing PAE and Scaffold4 that was heat-pressed (HM1-3-13) (solid lines).

Table 2 depicts the wet tensile strength (MPa), initial modulus (MPa), and elongation at the break (%) of various mycelium materials. Samples were made comparing PAE crosslinked samples at 1.5% dry weight percent in the panel with PAE crosslinked samples at 3% dry weight percent, and comparing PAE+pea protein crosslinked samples with samples crosslinked without pea protein.

TABLE 2

| | | Wet Tensile | | Initial Modulus | | Elongation at | |
|---|---|---|---|---|---|---|---|
| | | Strength (MPa) | | (MPa) | | the break (%) | |
| | | | Std. | | Std. | | Std. |
| n = ? | Sample | Average | Dev. | Average | Dev | Average | Dev. |
| 3 | 1.5% PAE, pea protein | 0.99 | 0.05 | 5.56 | 0.50 | 14.37 | 1.69 |
| | (HM25_5 min) | | | | | | |
| 3 | 1.5% PAE, pea protein | 0.87 | 0.23 | 4.40 | 0.53 | 16.10 | 2.61 |
| | (HM25_10 min) | | | | | | |
| 4 | 1.5% PAE, pea protein | 0.65 | 0.06 | 3.56 | 0.56 | 13.58 | 1.42 |
| | (HM1-3-1) | | | | | | |
| 5 | 3% PAE, pea protein | 1.03 | 0.30 | 5.64 | 1.12 | 12.88 | 0.90 |
| | (HM1-3-5) | | | | | | |
| 5 | 1.5% PAE, no pea protein | 0.86 | 0.17 | 3.48 | 0.74 | 14.46 | 1.81 |
| | (HM1-3-8) | | | | | | |

Additive Strengthening

Latex adhesive (leather glue) improved wet tensile strength and elongation at the break and did not impact the ability of PAE to crosslink mycelia (Table 3).

Table 3 depicts wet tensile strength (MPa), initial modulus (MPa), and elongation at the break (%) of various mycelium materials, comparing PAE crosslinked mycelia samples without glue with a PAE crosslinked mycelia sample with glue.

TABLE 3

| | | Wet Tensile | | Initial Modulus | | Elongation at | |
|---|---|---|---|---|---|---|---|
| | | Strength (MPa) | | (MPa) | | the break (%) | |
| | | | Std. | | Std. | | Std. |
| n = ? | Sample | Average | Dev. | Average | Dev | Average | Dev. |
| 3 | 1.5% PAE, pea protein | 0.99 | 0.05 | 5.56 | 0.50 | 14.37 | 1.69 |
| | (HM25_5 min) | | | | | | |
| 3 | 1.5% PAE, pea protein | 0.87 | 0.23 | 4.40 | 0.53 | 16.10 | 2.61 |
| | (HM25_10 min) | | | | | | |
| 4 | 1.5% PAE, pea protein | 0.65 | 0.06 | 3.56 | 0.56 | 13.58 | 1.42 |
| | (HM1-3-1) | | | | | | |
| 4 | 1.5% PAE, leather glue, | 1.18 | 0.22 | 5.50 | 0.35 | 20.53 | 2.33 |
| | pea protein (HM1-3-2) | | | | | | |
| 5 | 3% PAE, pea protein | 1.03 | 0.30 | 5.64 | 1.12 | 12.88 | 0.90 |
| | (HM1-3-5) | | | | | | |
| 5 | 1.5% PAE, no pea protein | 0.86 | 0.17 | 3.48 | 0.74 | 14.46 | 1.81 |
| | (HM1-3-8) | | | | | | |

Samples HM1-4-1 through HM1-4-5 included a dye, plasticizer, and scaffold, and were all pressed to about 1.4 mm.

Heat Pressing

Heat pressing the samples at 105° C. instead of crosslinking the samples in an oven at 105° C. resulted in a two-fold increase in wet tensile strength (Table 4).

Table 4 depicts wet tensile strength (MPa), initial modulus (MPa), and elongation at the break (%) of various mycelium materials. Samples were made comparing heat pressed samples at 105° C. (HM1-3-9p) with samples crosslinked at 105° C. in an oven.

TABLE 4

| | | Wet Tensile | | Initial Modulus | | Elongation at | |
|---|---|---|---|---|---|---|---|
| | | Strength (MPa) | | (MPa) | | the break (%) | |
| | | | Std. | | Std. | | Std. |
| n = ? | Sample | Average | Dev. | Average | Dev | Average | Dev. |
| 3 | 1.5% PAE, pea protein | 0.99 | 0.05 | 5.56 | 0.50 | 14.37 | 1.69 |
| | (HM25_5 min) | | | | | | |
| 4 | 1.5% PAE, pea protein | 0.65 | 0.06 | 3.56 | 0.56 | 13.58 | 1.42 |
| | (HM1-3-1) | | | | | | |
| 5 | 3% PAE, pea protein | 1.03 | 0.30 | 5.64 | 1.12 | 12.88 | 0.90 |
| | (HM1-3-5) | | | | | | |
| 4 | 1.5% PAE, leather glue, | 1.18 | 0.22 | 5.50 | 0.35 | 20.53 | 2.33 |
| | pea protein (HM1-3-2) | | | | | | |
| 5 | 1.5% PAE, pea protein, | 1.80 | 0.22 | 15.26 | 3.21 | 12.70 | 1.66 |
| | pressed (HM1-3-9p) | | | | | | |

Incorporating a Supporting Material

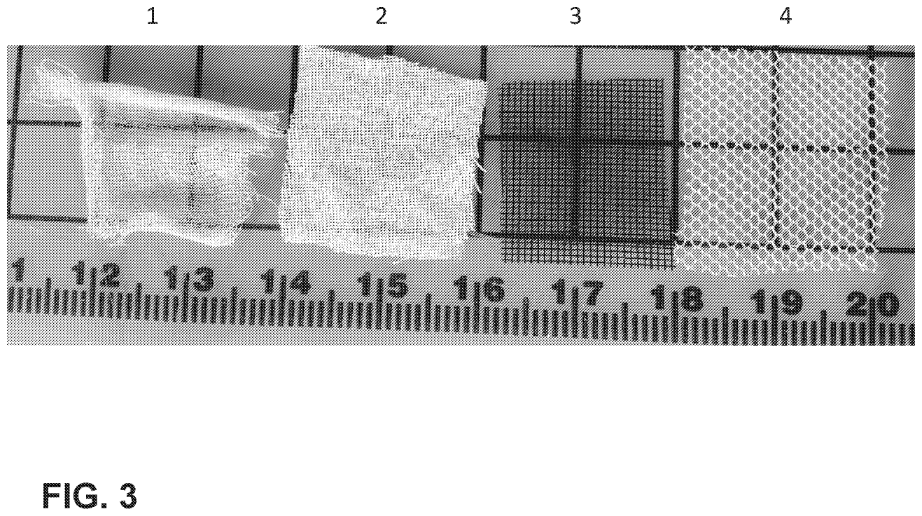

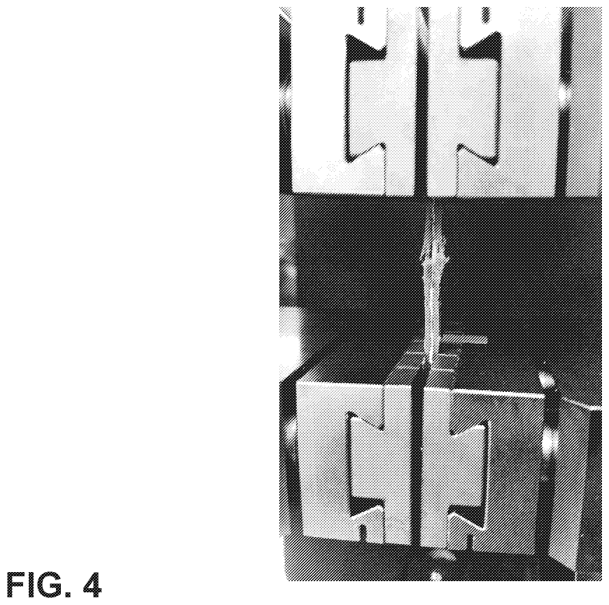

Incorporated supporting materials increased the wet tensile strength of mycelium materials, with stiffer supporting materials such as scaffolds yielding a higher initial modulus than less stiff supporting materials. In some samples, incorporated supporting materials increased the wet tensile strength of the overheated, pressed PAE samples about a two to five-fold increase. FIG. 3 shows different supporting materials incorporated inside the mycelium materials used herein. From left to right, FIG. 3 depicts a cheesecloth scaffold with pores slightly smaller than 1/16th of an inch (scaffold1); a cotton textile scaffold with pores smaller than 1/32 of an inch (scaffold2); a non-textile scaffold with pores 1/16th of an inch in size (scaffold 3); and a cotton textile scaffold with large pores ⅛th of an inch in size (scaffold4). FIG. 4 depicts scaffold 4 with Weldwood® adhesive after a wet tensile test.

Table 5 depicts the exemplary mechanical properties of four scaffolds used herein. Mechanical properties were tested on the Zwick system.

TABLE 5

| | | Tensile Strength | Initial Modulus | Elongation at the |
|---|---|---|---|---|
| n = ? | Sample | (MPa) | (MPa) | break (%) |
| 1 | Scaffold1 | 7.32 | 3.44 | 20.6 |
| 1 | Scaffold2 | 2.53 | 0.1 | 49 |
| 1 | Scaffold3 | 51.7 | 1110 | 4.96 |
| 1 | Scaffold4 | 8.7 | 2.32 | 31.1 |

An incorporated supporting material increased the wet tensile strength of one or more mycelium material samples, with stiffer supporting materials such as scaffolds taking more of the load prior to the disrupted mycelia breaking than less stiff scaffolds (e.g., scaffold3, non-textile, compared to scaffold2, cotton textile). Since one or more mycelium materials were relatively stiff, with a fairly low elongation at the break, a scaffold that was relatively stiff turned out to be more effective in yielding tensile property values comparable to bovine leather. It is desired for the supporting material to have a higher initial modulus than the mycelium material and optionally a lower elongation at the break so that the scaffold would initially take the strain from any tensile force and then break before the mycelium material breaks.

Scaffold3 (non-textile scaffold) met these desired requirements. Scaffold2 had a low initial modulus and high elongation at the break. Scaffold4 was made out of natural materials (cotton) and had a fair tensile strength and initial modulus. It was harder to tear Scaffold 4 as compared to Scaffold1.

Table 6 depicts the mechanical properties of pressed, crosslinked mycelium materials with and without an incorporated supporting material such as a scaffold. Mechanical properties of upholstery leather are used as a comparison.

TABLE 6

| | | Wet Tensile | | Initial Modulus | | Elongation at | |
|---|---|---|---|---|---|---|---|
| | | Strength (MPa) | | (MPa) | | the break (%) | |
| | | | Std. | | Std. | | Std. |
| n = ? | Sample | Average | Dev. | Average | Dev | Average | Dev. |
| 3 | Upholstery leather | 15.60 | 1.21 | 0.87 | 0.53 | 86.97 | 4.10 |
| 3 | Cultivated mycelium | 0.82 | 0.12 | 1.43 | 0.04 | 82.32 | 8.70 |
| | material, split | | | | | | |
| 5 | PAE, pea protein, pressed | 1.80 | 0.22 | 15.26 | 3.21 | 12.70 | 1.66 |
| | (HM1-3-9p) | | | | | | |
| 3 | PAE, Scaffold2, pressed | 2.97 | 0.10 | 16.97 | 4.05 | 13.47 | 1.61 |
| | (HM1-3-10) | | | | | | |
| 3 | PAE, Scaffold2, glue, pressed | 3.48 | 0.48 | 18.90 | 1.71 | 14.27 | 0.93 |
| | (HM1-3-6) | | | | | | |
| 3 | PAE, Scaffold2, glue, pressed | 4.46 | 0.43 | 15.33 | 4.80 | 18.20 | 1.01 |
| | (HM1-3-7) | | | | | | |
| 4 | PAE, Scaffold4, glue, pressed | 3.32 | 0.48 | 30.93 | 5.04 | 16.25 | 1.20 |
| | (HM1-3-11) | | | | | | |
| 4 | PAE, Scaffold3, pressed | 7.38 | 1.71 | 96.98 | 14.53 | 10.39 | 3.45 |
| | (HM1-3-12) | | | | | | |
| 4 | PAE, Scaffold4, pressed | 2.15 | 0.57 | 13.38 | 1.30 | 17.77 | 12.28 |
| | (HM1-3-13) | | | | | | |
| 4 | PAE, Scaffold4, glue, pressed | 5.91 | 1.48 | 39.68 | 3.53 | 20.20 | 1.85 |
| | (HM1-3-15) | | | | | | |

### Example 2: Slit Tear Strength of Mycelium Materials

Slit tear strength of composite mycelium materials was compared to that of intact cultivated mycelium material.

Table 7 depicts slit tear strength (N) and thickness (mm) of various mycelium materials.

TABLE 7

| | | | Slit tear | | | |
|---|---|---|---|---|---|---|
| | | | strength (N) | | Thickness (mm) | |
| | | | | Std. | | Std. |
| n = ? | Sample | Plasticized? | Average | Dev. | Average | Dev |
| 10 | Cultivated mycelium | Yes | 22 | 4 | 2.1 | 0.2 |
| | material, intact | | | | | |
| 10 | Bovine leather | Yes | 106 | 9 | 1.29 | 0.02 |
| 2 | HM1-1-1 | No | 29 | 3 | 2.4 | 0.1 |
| 2 | HM1-1-7 | No | 51 | 4 | 3.22 | 0.07 |
| 1 | HM1-1-11 | Yes | 13 | | 1.75 | |
| 1 | HM1-1-11_120p | Yes | 18 | | 0.96 | |
| 1 | HM0 | Yes, 5% glycerol | 7 | | 2.2 | |
| 2 | HM1-4-3 | Yes, 5% glycerol | 42 | 2 | 1.4 | 0.2 |

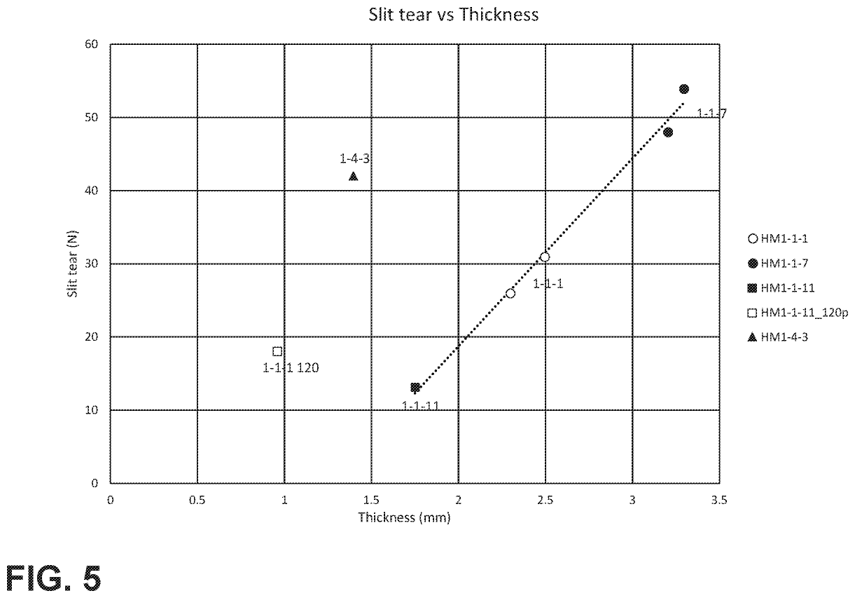

The slit tear strength of various mycelium materials ranged from about 7N to about 50N. FIG. 5 depicts a plot of slit tear versus thickness of various mycelium materials, including pressed samples (HM1-4-3 and HM1-1-11_120 p) and unpressed samples. The slit tear strength of pressed samples was far stronger than that of unpressed samples. HM1-4-3 was pressed in the presence of 1.5% PAE and HM1-1-11_120 p was pressed in the presence of epoxidized soybean oil. Unpressed samples without PAE had slit tear strengths that behaved linearly with thickness.

### Example 3: Alignment of Mycelium Hypha from Ganoderma sessile

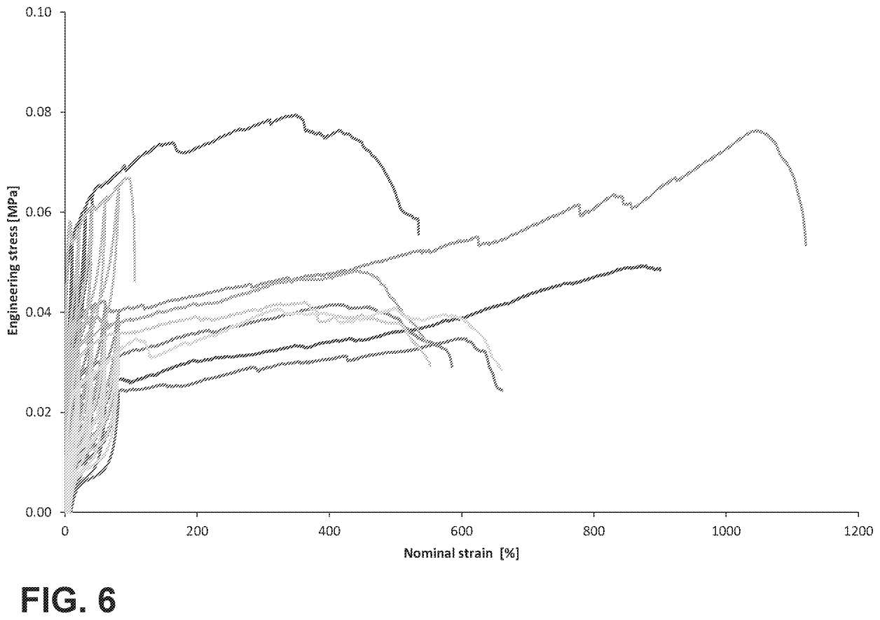

Next, the cultivated mycelium material or composite mycelium material was disrupted by physically aligning branching hyphae in one or more directions. FIG. 6 shows a stress-strain curve plotting through-thickness drafting stress as a function of the strain to aligned mycelium. The strain cycles were performed from 10% to 80%, in increments of 10%, before drafting to the maximum elongation. The force was measured while the masses of branching hyphae were being aligned. The curves illustrated a proportional limit followed by a maximum in the curve at which necking takes place.

Table 8 depicts the drafting maximum alignment stress and elongation range for the through-thickness drafting illustrated in FIG. 6.

TABLE 8

| | Minimum | Maximum |
|---|---|---|
| Maximum alignment | 0.035 | 0.079 |
| stress (MPa) | | |
| Elongation at the break | 105.3 | 1120.5 |
| (%) | | |

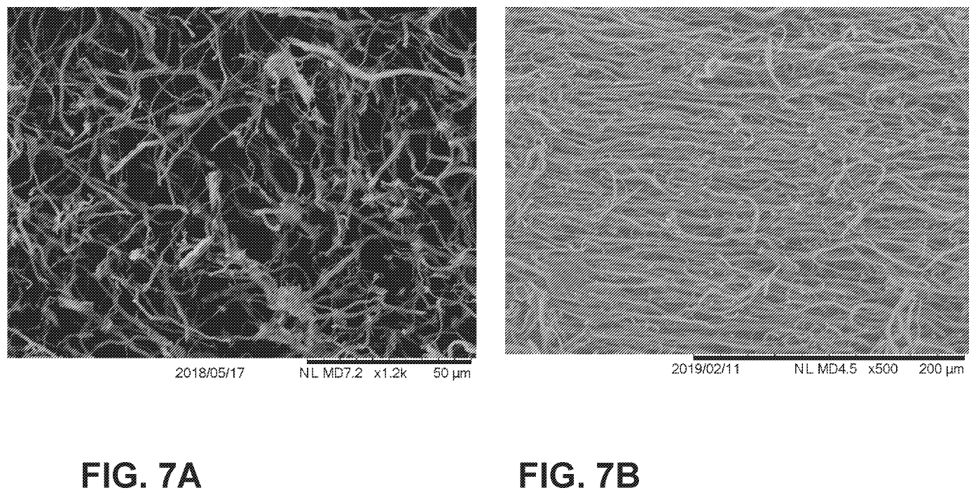

FIGS. 7A and 7B show SEM micrographs of mycelium hyphae before drafting (FIG. 7A) and after drafting (FIG. 7B). In this embodiment, the fibers were aligned along the stress direction. The lamina included three successive layers.

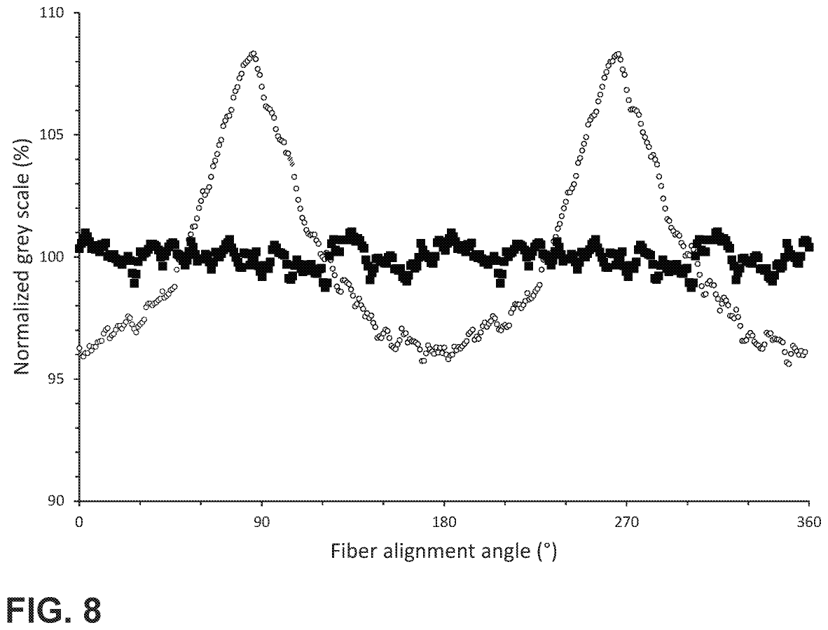

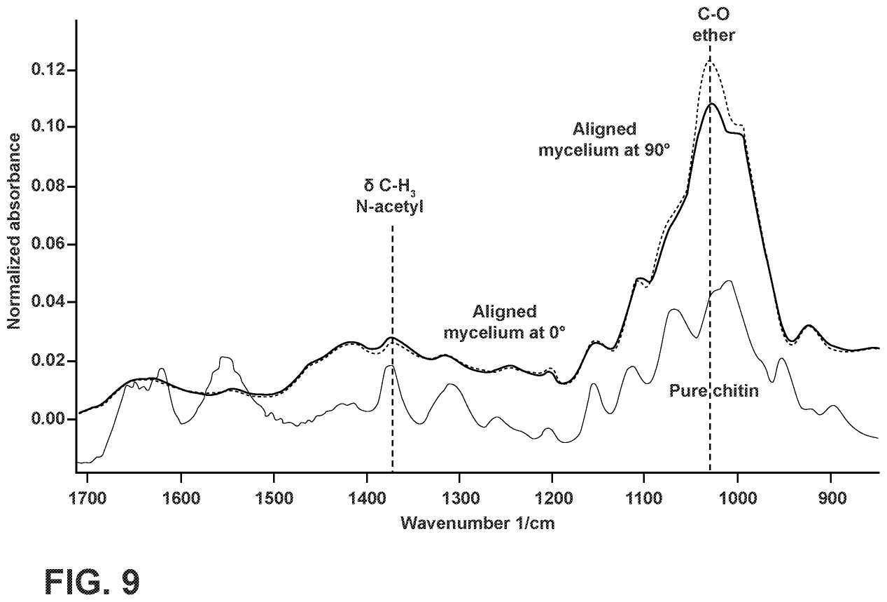

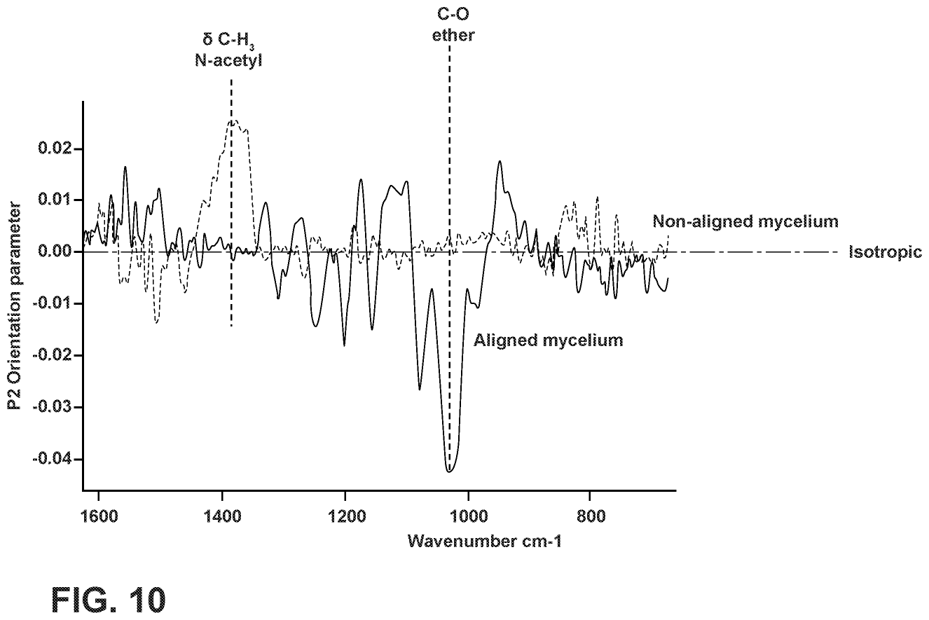

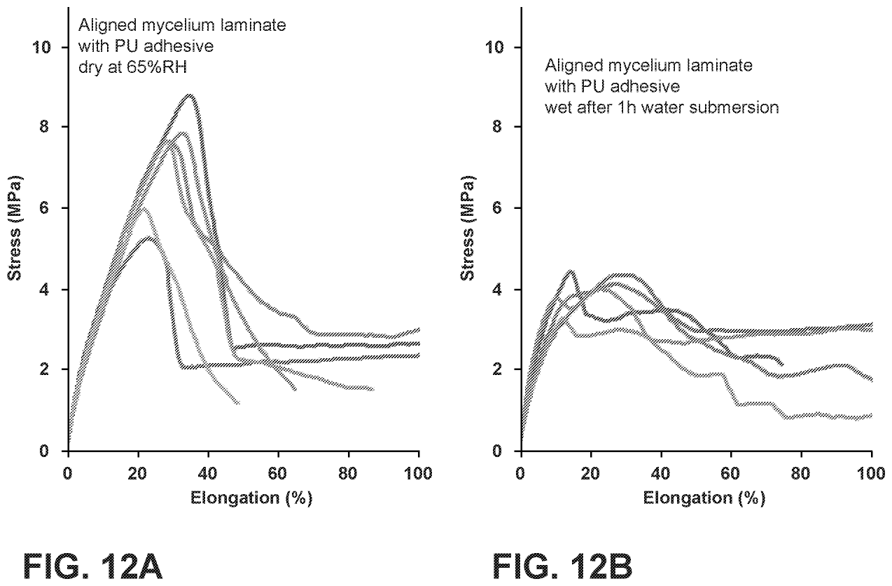

FIG. 8 shows a Fourier transform graph of the mycelium SEM image before drafting (black squares) and after drafting (grey circles). The graph illustrated normalized grey scale (%) as a function of fiber alignment angle. FIG. 9 shows polarized FTIR spectra of aligned mycelium hyphae along with the polarization (0 degrees) and perpendicular to the polarization (90 degrees). A spectrum of pure chitin is shown as a comparison. FIG. 10 shows a second Legendre order parameter (<P2>) as a function of the wavenumber for non-aligned and aligned mycelium hyphae. The graph demonstrated there was an alignment of hyphae at particular frequencies. FIGS. 11A and 11B show SEM micrographs of two laminae of aligned mycelium bonded with polyurethane hot melt adhesive at 150× (FIG. 11A) and 500× (FIG. 11B) magnification. The surface of the layer was measured. FIGS. 12A and 12B show stress-strain curves for aligned mycelium and aligned mycelium bonded with polyurethane hot melt adhesive tested after conditioning at 65% RH at a dry state (FIG. 12A) and a wet state (FIG. 12B).

Table 9 depicts tensile properties of aligned mycelium and aligned mycelium bonded with polyurethane (PU) hot melt adhesive tested after conditioning at 65% relative humidity (RH) and after a one-hour water submersion.

TABLE 9

| | | | | Initial modulus | | Yield strength | | | |
|---|---|---|---|---|---|---|---|---|---|
| | | Thickness (mm) | | (MPa) | | 0.2% (MPa) | | Strength (MPa) | |
| | | Min | Max | Min | Max | Min | Max | Min | Max |
| Drafted mycelium | Dry | 0.546 | 0.688 | 49.266 | 63.05 | 0.9505 | 1.538 | 5.750 | 8.804 |
| PU laminate | Wet | 0.440 | 0.500 | 41.094 | 74.94 | 0.8054 | 1.791 | 3.298 | 4.549 |
| Drafted mycelium | Dry | 0.066 | 0.162 | 0.253 | 47.04 | 0.0562 | 7.940 | 4.195 | 10.730 |
| lamina | Wet | 0.141 | 0.148 | 7.956 | 9.801 | 0.2942 | 0.328 | 0.857 | 0.935 |

Dry tensile strength of mycelium material was measured against wet tensile strength. For instance, drafted mycelium PU laminate yielded dry tensile strength of 5.750 MPa to 8.804 MPa and a wet tensile strength of 3.298 MPa to 4.549 MPa. It was observed that initial modulus dropped due to wetting. There was likewise a larger decrease in values that occurred for non-bonded laminate as compared to bonded laminate. In the drafted mycelium lamina samples without PU, absence of a bonding agent did not change the tensile strength. In addition, using polyurethane yielded a material that was four times stronger when wet. Without intending to be bound by any particular theory, the tensile strength properties may be dependent on the specific type of manipulations of hyphae.

### Example 4: Hydroentanglement of Mycelium Materials

The components for the materials were disrupted in a blender and the resulting slurry included at least cultivated mycelium material and water. Water was directed at the mycelia slurry at about 1000 psi through pores with a diameter of about 50 microns. The mycelia slurry was submerged within a solution including one or more bonding agents. Without intending to be bound by any particular theory, it is proposed that the one or more masses of branching hyphae of the mycelium material entangles effectively via hydroentanglement, leading to certain mechanical property improvements in performance, e.g., wet tensile strength, an initial modulus, an elongation percentage at the break, a thickness, and/or a slit tear strength.

### Example 5: Liquid Cultivation Process of Mycelium

Table 10 depicts exemplary parameters for cultivating mycelium in a liquid process for 2 different fungal strains. The mycelium was cultivated in a benchtop bioreactor having a 2 L glass vessel. The inoculum for both strains was grown as indicated in Table 10 and cell banked by blending the mycelia in 25% glycerol and 3.5% milk. The inoculum was stored at −80° C.

TABLE 10

| | | Phycomyces blakesleeanus | Neurospora crassa |
|---|---|---|---|
| Strain | Description | Zygomycota. Carotenoid | Ascomycota. Non- |
| | | producer. Formed sporangia | conidiating strain. |
| | | at water/air interface. | |
| Inoculum | Growth | Scraped from glycerol vial | Scraped from glycerol vial |
| | Method | and added to Falcon tube | and added to shake flask |
| | | PDA¹ (solid). | with PDA¹ (solid). |
| | | Germinated at 48° C. for | Transferred liquid to |
| | | 10-15 min.; grow at room | shake flask with YM media |
| | | temperature. | and grown for 24 hrs at |
| | | Incubated in shake flasks | 30°C., 150 rpm. |
| | | at 25° C. and 60 rpm with | Disrupted inoculum in |
| | | YM media for 4-5 days. | blender. |
| Bioreactor | Media | Vogels media with 0.67 g/L | Vogels media with biotin. |
| liquid | | thiamine. | |
| process | Conditions | Media at pH 5 and 25° C. | Media at pH 5 and 30° C. |
| | | Aeration 1 VVM². Impeller | Aeration 1 VVM². Impeller |
| | | at 100 rpm or no agitation. | at 200 rpm. Batch was fed |
| | | Batch was fed with 50% | with 50% glucose and |
| | | glucose and NH₄OH. | NH₄OH. |
| | Biomass | 10 g/L in 7 days. | 10 g/L in 4 days. |
| | (DCW) | | |

¹PDA: potato dextrose agar.
²VVM: volume of air per volume of medium per unit of time (liter/liter/minute).

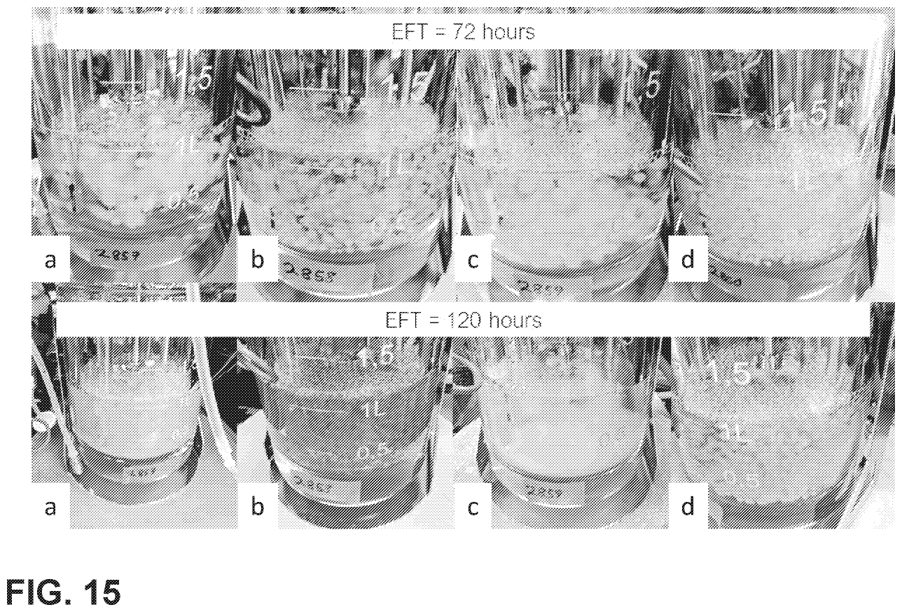

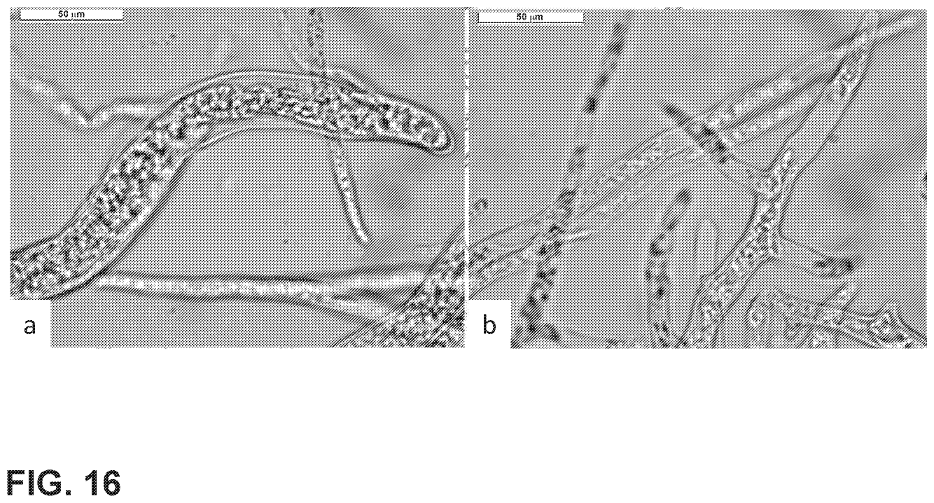

FIG. 15 shows photographs of exemplary Phycomyces blakesleeanus biomasses grown in a liquid process according to Table 10. Panels (a) and (b) illustrate exemplary sample Phycomyces blakesleeanus biomass grown without agitation at 72 hours and 120 hours incubation in the bioreactor. Panels (c) and (d) illustrate exemplary sample Phycomyces blakesleeanus biomasses grown with agitation using an impeller rotating at 100 rpm at 72 hours and 120 hours incubation in the bioreactor. FIG. 16 shows photographs of exemplary Phycomyces blakesleeanus hyphae grown according to the liquid process of Table 10. Generally, the exemplary Phycomyces blakesleeanus biomasses consumed about 15 g/L glucose in about 4 days with a total consumed glucose of about 40 g/L corresponding to about a 40% biomass yield on glucose.

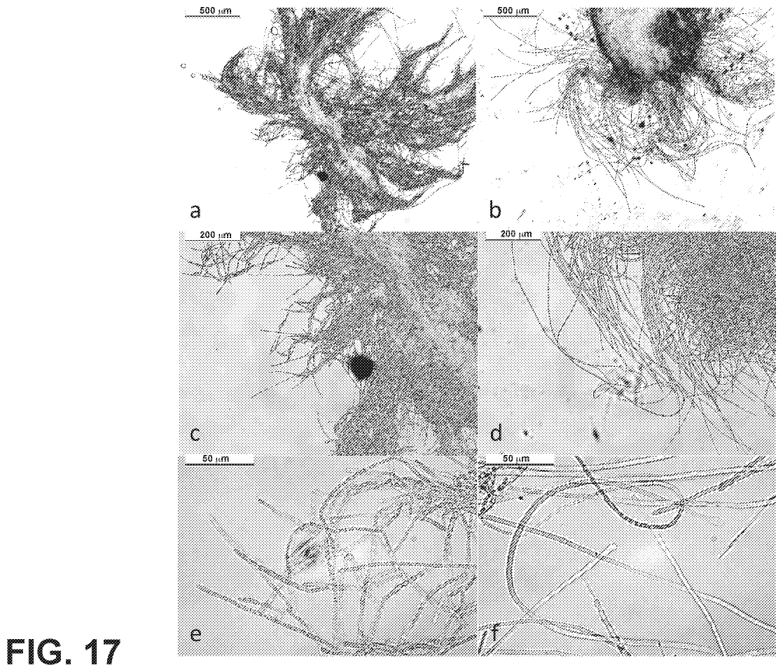

FIG. 17 shows photographs of exemplary Neurospora crassa hyphae grown according to the liquid process of Table 10. FIG. 17 generally shows hairy clumps of hyphae. The hyphae were generally unbranched and several millimeters in length. Generally, the exemplary Neurospora crassa biomasses consumed about 10 g/L glucose with a total consumed glucose of about 50 g/L after 4 days corresponding to about a 20% biomass yield on glucose.

### Example 6: Hydroentanglement of Liquid Process Grown Biomasses

Table 11 depicts wet tensile strength, initial modulus, and elongation at break for various mycelium materials with and without treatment in a hydroentanglement process.

TABLE 11

| | | Wet Tensile strength | | Initial Modulus | | Elongation at the | |
|---|---|---|---|---|---|---|---|
| | | (MPa) | | (MPa) | | break (%) | |
| | | | Std. | | Std. | | Std. |
| n | Sample | Average | Dev. | Average | Dev | Average | Dev |
| 3 | Hydroentangled | 0.15 | 0.02 | 0.45 | 0.41 | 154 | 17 |
| | Ganoderma | | | | | | |
| 3 | Mycelia + Fiber | 0.12 | 0.03 | 0.94 | 0.10 | 69 | 18 |
| | Additive | | | | | | |
| 4 | Hydroentangled | 0.24 | 0.04 | 1.06 | 5.00 | 39 | 16 |
| | Phycomyces | | | | | | |
| 3 | Mycelia - Not | 0.25 | 0.04 | 4.62 | 0.40 | 11 | 1 |
| | Hydroentangled | | | | | | |

The following samples were used to obtain the data in Table 11:

Hydroentangled Ganoderma: Ganoderma sessile aerial mycelia were blended in a blender. The mycelia were wet-laid using a vacuum-assisted wet-lay system, and hydroentangled according to the hydroentanglement process described below. The wet-laid sample was 10 g of mycelium material in a 6×6 inch mat.

Mycelia+Fiber Additive: Ganoderma sessile aerial mycelia were prepared using the same method as the Hydroentangled Ganoderma except for the addition of 10 wt % of a 10 mm pre-cut TENCEL™ fiber.

Hydroentangled Phycomyces: liquid cultivated Phycomyces blakesleeanus mycelia blended in a blender, wet-laid using a vacuum-assisted wet-lay system, and hydroentangled according to the hydroentanglement process described below.

Mycelia—Not Hydroentangled: liquid cultivated mycelia were blended in a blender and wet-laid using a vacuum-assisted wet-lay system. No hydroentanglement treatment was performed.

### Hydroentanglement

The hydroentanglement process according to the present disclosure included preparing a slurry of the mycelium and wet-laying the sample to form a mat. The spinneret used in the hydroentanglement process included holes having a diameter of 50 μm. A metal screen was placed on top of a vacuum-assisted wet-lay system. A forming cloth was placed on top of the metal screen and a silicone mask having the desired mat-forming dimensions was placed on top of the forming cloth. The appropriate amount of mycelium material based on the dimensions of the mat was weighed out (e.g. about 11.25 grams for a 6×6 inch mat). The mycelia could optionally be manually broken up prior to blending in the blender. Water was added to the mycelia to form a 1-2 wt % slurry and the material was blended. Vacuum was applied to the wet-lay system and about ⅓ of the slurry was poured onto the forming cloth. The spinneret was moved over the mostly dry wet-lay in a “Z” or “N” shaped pattern, ensuring that the turn takes place over the silicone mask. The spinneret was moved over the sample when the jet pressure was in the range of about 750 psi to about 800 psi and a flow rate of about 100 mL/min. to about 230 mL/min. The spinneret was continuously moved during operation to avoid over-soaking any particular area. A slight overlap was provided for each pass with the spinneret and the entire mat was covered for two lengths (down and back) until continuing the pattern with a 90° rotation. The process was then repeated for two lengths. The next ⅓ of the slurry was poured onto the forming cloth and the spinneret pattern was repeated, followed by the final ⅓ of the slurry and repetition of the spinneret pattern.

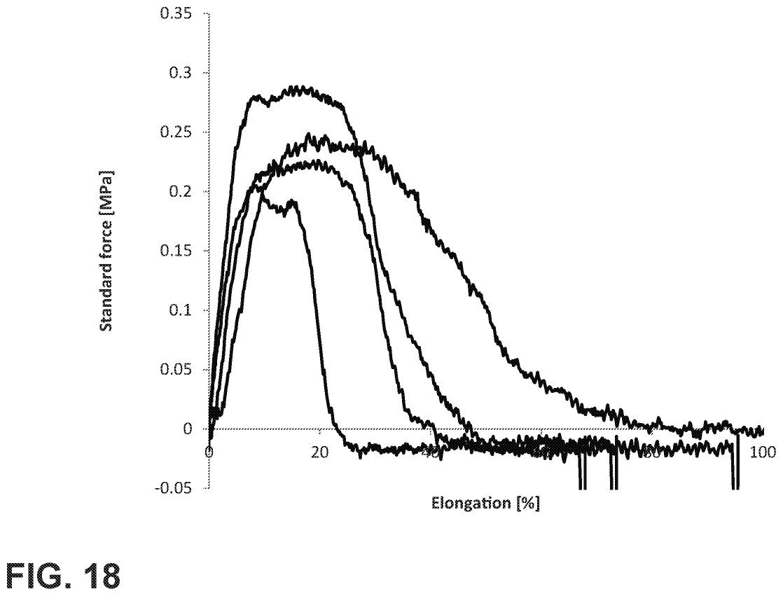

Drying the hydroentangled mat included restraining the sample between two drying racks and drying the sample in a convection oven at about 40.5° C. to about 45° C. for about 3 hours. The mat was then carefully removed from the forming cloth. The samples were tested for wet tensile strength, initial modulus, and elongation at break as described herein. FIG. 18 shows a plot of wet tensile stress-strain curve of Phycomyces blakesleeanus grown in liquid culture, blended, then wet-laid and hydroentangled. Engineering stress (MPa) is plotted against nominal strain (%) and the strain cycles from 10% to 80% in increments of 10% before drafting to the maximum elongation.

As demonstrated by the data shown in Table 11, samples which were hydroentangled exhibited a higher elongation at break than samples that were not hydroentangled.

### Example 7: Neurospora crassa and PAE Bonding Agent

Table 12 depicts wet tensile strength, initial modulus, and elongation at break for various mycelium materials with and without polyamide-epichlorohydrin resin (PAE) bonding agent treatment.

TABLE 12

| | | Wet Tensile strength | | Initial Modulus | | Elongation at the | |
|---|---|---|---|---|---|---|---|
| | | (MPa) | | (MPa) | | break (%) | |
| | | | Std. | | Std. | | Std. |
| n | Sample | Average | Dev. | Average | Dev | Average | Dev |
| 3 | Aerial mycelium | 0.11 | 0.0 | 1.97 | 0.78 | 8.20 | 0.11 |
| | material | | | | | | |
| 3 | Liquid cultivated | 0.13 | 0.01 | 0.96 | 0.15 | 13.03 | 1.08 |
| | Neurospora, no PAE | | | | | | |
| 5 | Aerial mycelium | 0.86 | 0.17 | 3.48 | 0.74 | 14.46 | 1.81 |
| | material + PAE | | | | | | |
| 4 | Liquid cultivated | 1.10 | 0.39 | 2.04 | 2.99 | 14.75 | 6.02 |
| | Neurospora + PAE | | | | | | |
| 4 | Liquid cultivated | 2.29 | 0.39 | 10.47 | 1.21 | 13.75 | 1.48 |
| | Neurospora:Aerial | | | | | | |
| | mycelium material + | | | | | | |
| | PAE | | | | | | |

The following samples were used to obtain the data in Table 12:

Aerial mycelium material: Ganoderma sessile aerial mycelia were blended in a blender, and wet-laid via gravity through a porous substrate.

Liquid cultivated Neurospora, no PAE: liquid cultivated Neurospora crassa fluffy mycelia were wet-laid via gravity through a porous substrate. No PAE treatment was performed.

Aerial mycelium material+PAE: Mycelia. Samples with PAE included 1.5 wt % PAE and were heat-treated at 105° C. for 5 minutes.

Liquid cultivated Neurospora:Aerial mycelium material+PAE: PAE-treated 50:50 by weight Neurospora crassa:Aerial mycelium material. Samples with PAE included 1.5 wt % PAE and were heat-treated at 105° C. for 5 minutes.

The results of Table 12 show that the liquid cultivated Neurospora crassa can be used to form a mycelium material having a wet tensile strength, initial modulus, and elongation at break that was similar in magnitude to the Aerial mycelium material+PAE. The data of Table 12 also shows that the inclusion of a bonding agent with Neurospora crassa, such as PAE, can increase the wet tensile strength and initial modulus of the material compared to a material formed without PAE.

### Example 8: RMs2374 Neurospora crassa Growth & Fermentation

Spores of RMs2374 N. crassa were used to inoculate a 2 L benchtop bioreactor to produce a filamentous biomass of dispersed mycelia in a liquid growth process according to aspects of the present disclosure.

Seed Train and Inoculation

Spores from a stock were added to seed flasks containing Vogel's media and agar and incubated at 30° C. for 3 days. The seed flasks were then transferred to room temperature (about 25° C.) and stored for 1 week (stationary), during which conidia formed. Once clumps or chains of conidia were visible, a sterile loop was used to harvest the conidia into a sterile 0.2 wt % Tween20 solution. The solution was gently mixed to disperse the spores and then the solution was strained through a 40 μm sterile mesh to remove chunks of mycelia. Spore concentration ranged from about 10⁶ to about 10⁸ spores/mL, depending on growth in the seed flask and the volume of solution. The spores in solution were injected directly into the benchtop bioreactor without freezing or refrigeration of the spores.

Incubation and Growth of RMs2374 N. crassa Filamentous Biomass

The spore solution was added to a 2 L benchtop bioreactor containing 1.8 L of modified Vogel's media to obtain a concentration of about 10⁵ spores/mL. The spores were incubated in the bioreactor for about 48 hours while the temperature of the media was maintained at about 30° C. The airflow in the bioreactor was about 0.5 vvm (L/L/min.) and the pressure was about 5-15 prig. Table 13 lists the components of the modified Vogel's media used to grow the filamentous biomass and the initial concentration of each component, i.e., the concentration of each component at the beginning of the incubation time period. A Vogel's 50× salt solution was prepared (as given in Microbial Genetics Bulletin vol. 13, pg. 42-43 (1956)), and diluted with deionized water to provide the desired volume of 1× salt solution and then steam sterilized. Trace metals, biotin, and glucose were added as post-sterile additions. Sterile polypropylene glycol 2000 was added to provide a final concentration of 1 mL/L.

TABLE 13

| Component | Initial Concentration | |
|---|---|---|
| Na-Citrate | 2.5 | g/L |
| KNO₃ | 2.5 | g/L |
| (NH₄)H₂PO₄ | 2.88 | g/L |
| KH₂P0₄ | 1.6 | g/L |
| MgSO₄—7 H₂O | 0.2 | g/L |
| CaCl₂—2 H₂O | 0.1 | g/L |
| Citric Acid-H₂O | 0.005 | g/L |
| ZnSO₄—7 H₂O | 0.005 | g/L |
| Fe(NH₄)₂(SO₄)₂—6 H₂O | 0.001 | g/L |
| CuSO₄—5H₂O | 0.00025 | g/L |
| MnSO₄—H₂O | 0.00005 | g/L |
| H₃BO₃ | 0.00005 | g/L |
| Na₂MoO₄—2 H₂O | 0.00005 | g/L |
| Biotin | 0.000005 | g/L |
| Glucose | 10 | g/L |
| Polyglycol P 2000 | 1 | mL/L |

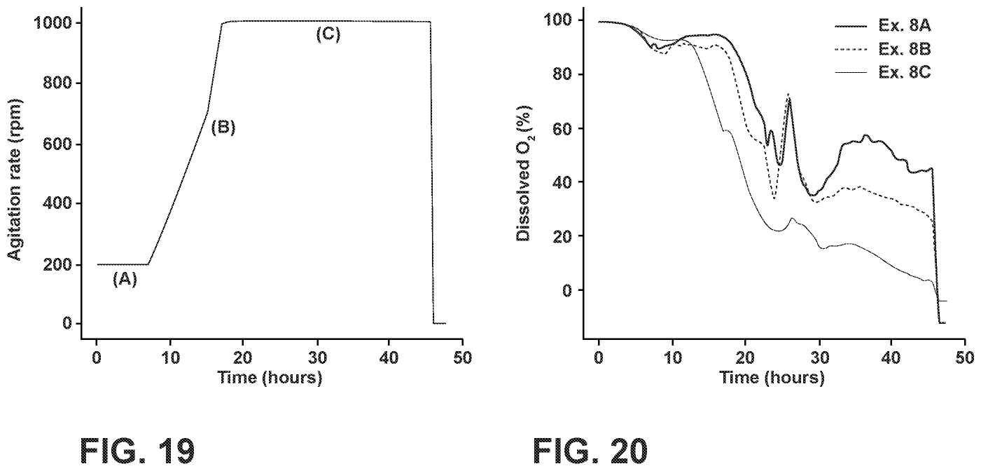

During the incubation period, the media was agitated using 6-blade Rushton turbine impellers according to the agitation profile illustrated in FIG. 19. As illustrated in FIG. 19, the mixture was agitated in an initial agitation stage (A) at a low agitation rate (about 200 rpm) to facilitate the initial growth of the spores and formation of dispersed hyphae. Following the initial agitation stage (A), the mixture was agitated according to a ramp profile during a ramp stage (B). During the ramp stage (B), the agitation rate was increased from the initial stage (A) to a final stage (C). The agitation rate was generally held steady during the final stage (C) until the end of the incubation period, which was about 48 hours. FIG. 20 illustrates the measured dissolved oxygen values during the incubation period for three identical sample runs, Examples 8A-8C (Ex. 8A-8C). In each of the sample runs Ex. 8A-8C, the level of dissolved oxygen decreased over time as the biomass grew. At around 8 hours, an initial decrease in the level of dissolved oxygen was observed. This initial decrease in the level of dissolved oxygen generally corresponded with initiation of the ramp stage (B). Without wishing to be limited by any theory, it is believed that decreased oxygen levels can result in slow and/or delayed growth and may affect the morphology of the mycelia biomass. It is believed that the increase in agitation of the media over time during the ramp stage (B) can compensate at least in part for the decreased oxygen levels to at least partially attenuate the slowed/delayed growth caused by the decreasing levels of dissolved oxygen. During the ramp stage (B), the agitation rate continued to increase based on the continued decrease in dissolved oxygen. Around 20 hours, the agitation rate reached the final stage (C) and was maintained until the end of the incubation period. During the final stage (C) of the agitation profile, the agitation rate was generally maintained at a high rate to facilitate maintaining the dissolved oxygen levels above about 20% for as long as possible, without damaging the morphology of the biomass and/or disrupting the contents and/or components of the bioreactor.

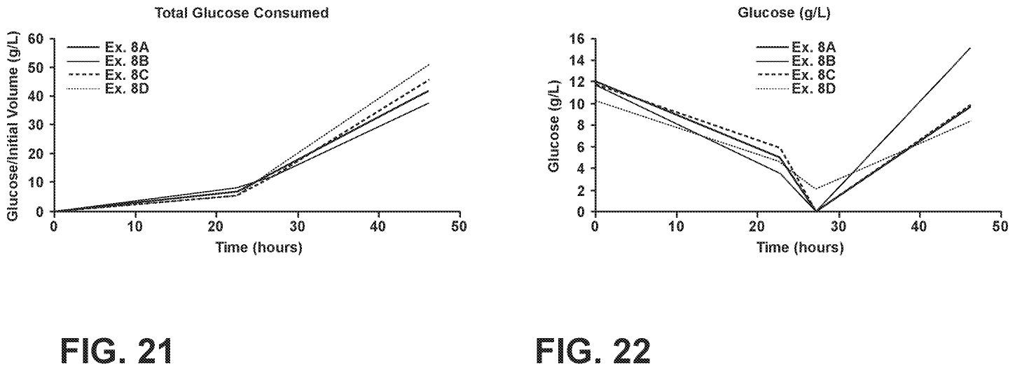

At around the 24 hour time point in the incubation period, glucose was fed into the bioreactor at a constant rate of about 1.8 grams of glucose/L/hr. Without wishing to be limited by any theory, it is believed that by about 24 hours the initial glucose supplied with the modified Vogel's media has been consumed. The consumption of the initial glucose amount is considered to generally correspond with a spike in the dissolved oxygen content of the media, which is visible in FIG. 20 for Ex. 8A-8C. During this phase of the incubation, the biomass continued to grow. FIGS. 21 and 22 illustrate plots of the total glucose consumed and the concentration of glucose present in the mixture, respectively, as a function of time. FIGS. 21 and 22 illustrate data for four identical sample runs, Ex. 8A-8D. As illustrated in FIG. 21, as the biomass grows, the amount of glucose consumed increases. As illustrated in FIG. 22, the initial amount of glucose can be seen to decrease toward 0 by about 24 hours as the biomass grows; at 24 hours the feed of supplemental glucose was turned on, as can be seen by the increase in glucose content. FIGS. 21 and 22 also demonstrate the reproducibility of the growth process for RMs2374 N. crassa.

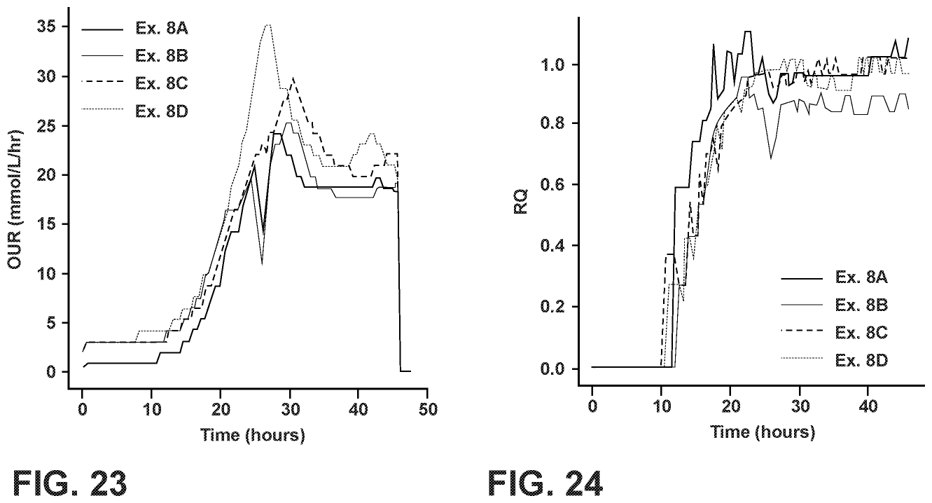

FIGS. 23 and 24 illustrate plots of the oxygen uptake rate (“OUR”) and the respiratory quotient (“RQ”) as a function of time for Ex. 8A-8D. The respiratory quotient RQ is determined in the conventional manner as the carbon dioxide evolution rate (CER) divided by the oxygen uptake rate OUR (RQ=CER/OUR). The data in FIGS. 23 and 24 are illustrative of the reproducibility of the growth characteristics of RMs2374 N. crassa according to aspects of the present disclosure, which can be important in manufacturing settings. The growth rate μ was estimated to be greater than about 0.25 hr⁻¹ (μ>˜0.25 hr⁻¹) during the exponential phase for Ex. 8A-8D, based on the RQ data. The growth rate μ was calculated using the standard mathematical formula for continuous exponential growth and decay. The time range for calculating the growth rate was selected using a mathematical script that calculates the growth rate in a range from when detected CO₂ (%) is between certain limits (e.g., between 0.1% and 1%) well within the exponential part of the growth curve.

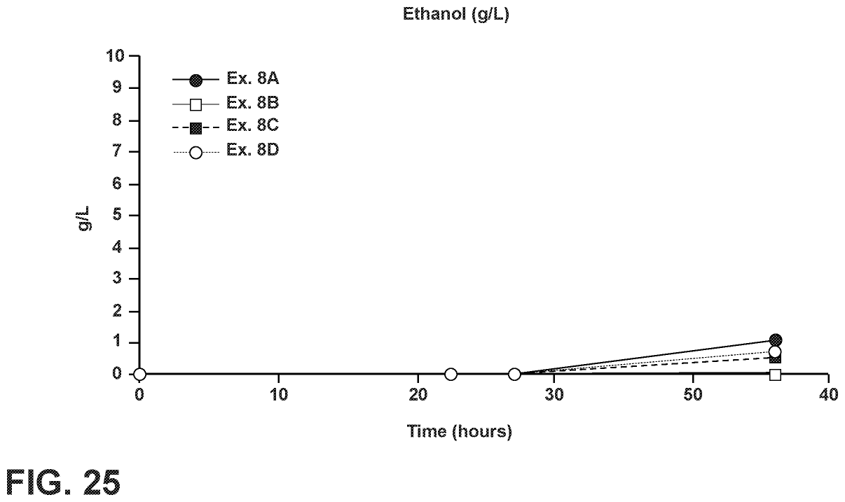

FIG. 25 illustrates a plot of the ethanol content in the vessel as a function of time. Ethanol accumulation was observed in some of the sample runs, such as Ex. 8A, Ex. 8C, and Ex. 8D, and was considered to generally be correlated with oxygen limitation within the vessel.

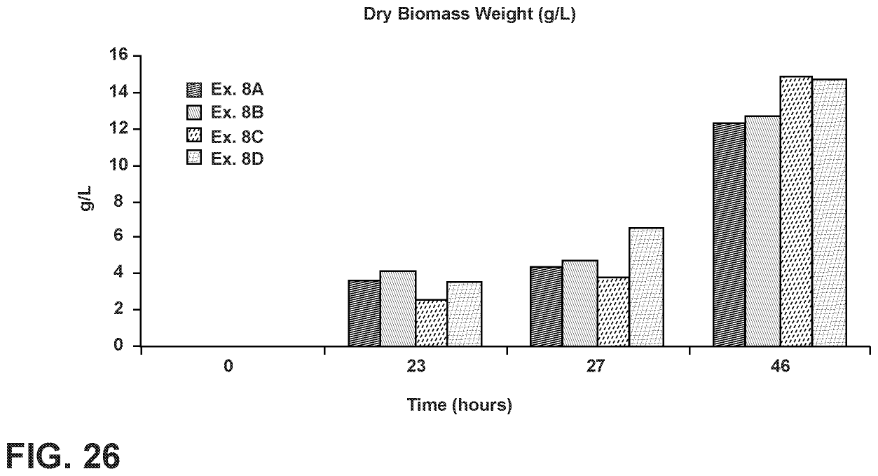

FIG. 26 illustrates the dried weight of biomass material collected for Ex. 8A-8D after incubation periods of about 23 hours (“23 hrs”), about 27 hours (“27 hrs”), and about 46 hours (“46 hrs”). The dried weight of biomass material for each sample was determined based on extracting a 10 mL broth aliquot from the vessel and filtering, washing, and drying the biomass to estimate the biomass at each time point. The broth aliquot was filtered through a 600 μm mesh screen and washed with water 3 times. The data in FIG. 26 illustrates the reproducibility of the growth characteristics of RMs2374 N. crassa according to the aspects of the present disclosure, which, as noted previously, can be important in manufacturing settings.

The incubation period was ended at 48 hours and the biomass was harvested from the vessel, filtered through a 600 μm mesh screen and washed with water 3 times. The volume of water used in each wash corresponded to the full tank volume (e.g., the biomass was washed with 2 L of water, 3 times). The biomass is dewatered to roughly 10% solids content and then preserved by freezing or drying.

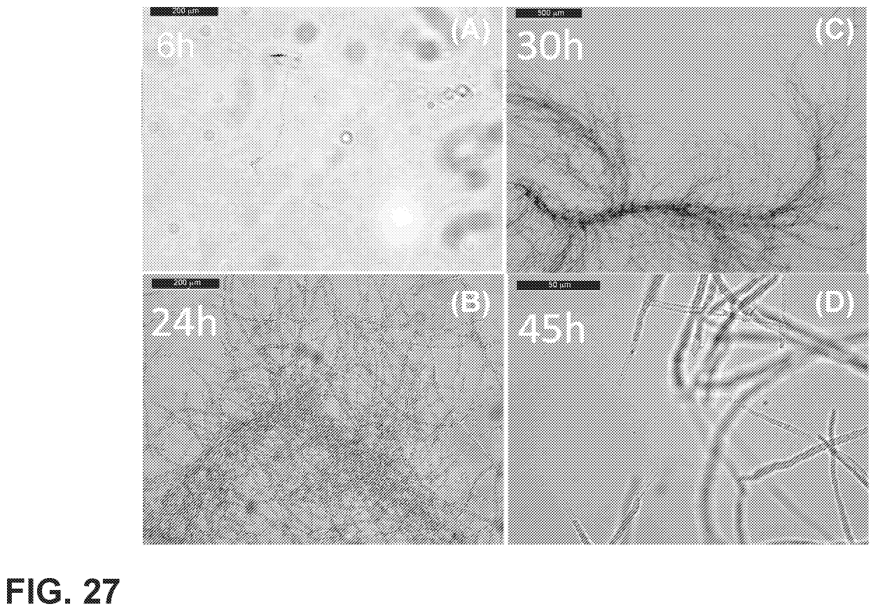

FIG. 27 shows micrograph images of RMs2374 N. crassa for one of the examples at different time periods. Inset image (A) in FIG. 27 shows germination of RMs2374 N. crassa spores at 6 hours in the seed flask. Inset image (B) shows strand intertwining after incubating in the bioreactor for 24 hours. Inset image (C) shows RMs2374 N. crassa macrostructure after incubating for 30 hours. Inset image (D) shows fragmented mycelia after 48 hours of incubation. The results for Example 8 demonstrate the RMs2374 N. crassa to reproducibly generate dispersed mycelia having filament lengths of at least 200 μm in amounts of about 12-15 g/L or more in a 2 L bioreactor after an incubation period of about 48 hours in a liquid growth process according to the present disclosure.

### Example 9: RMs2374 Neurospora crassa Fermentation

Spores of RMs2374 N. crassa were used to inoculate a 2 L benchtop bioreactor to produce a filamentous biomass of dispersed mycelia in the same manner as described above in Example 8. The spores were incubated in a bioreactor under the same conditions as described above for Example 8, except that the modified Vogel's media (Table 13) included 1 mL/L of TERGITOL™ L-81 surfactant instead of Polyglycol P 2000. These conditions also produced dispersed mycelia having filament lengths of at least 200 μm in amounts of about 12-15 g/L or more in a 2 L bioreactor after an incubation period of about 48 hours in a liquid growth process according to the present disclosure.

The following non-limiting aspects are encompassed by the present disclosure. To the extent not already described, any one of the features of the first through the one hundred fifty-sixth aspect may be combined in part or in whole with features of any one or more of the other aspects of the present disclosure to form additional aspects, even if such a combination is not explicitly described.

According to a first aspect of the present disclosure, a composite mycelium material, includes: a cultivated mycelium material including one or more masses of branching hyphae, wherein the one or more masses of branching hyphae is disrupted; and a bonding agent.

According to a second aspect of the present disclosure, the composite mycelium material of aspect 1, wherein the cultivated mycelium material has been generated on a solid substrate.

According to a third aspect of the present disclosure, the composite mycelium material of aspects 1 or 2, wherein the cultivated mycelium material includes one or more masses of disrupted branching hyphae.

According to a fourth aspect of the present disclosure, the composite mycelium material of any one of aspects 1 to 3, wherein the one or more masses of disrupted branching hyphae has a length of 0.1 mm to 5 mm.

According to a fifth aspect of the present disclosure, the composite mycelium material of aspect 4, wherein the one or more masses of disrupted branching hyphae has a length of 2 mm.

According to a sixth aspect of the present disclosure, composite mycelium material of any one of aspects 1 to 5, wherein the composite mycelium material further includes a supporting material.

According to a seventh aspect of the present disclosure, the composite mycelium material of aspect 6, wherein the supporting material has a pore size of 1/16th of an inch.

According to an eighth aspect of the present disclosure, the composite mycelium material of aspect 6, wherein the supporting material includes a reinforcing material.

According to a ninth aspect of the present disclosure, the composite mycelium material of aspect 8, wherein the reinforcing material is entangled within the composite mycelium material.

According to a tenth aspect of the present disclosure, the composite mycelium material of aspect 6, wherein the supporting material includes a base material.

According to an eleventh aspect of the present disclosure, the composite mycelium material of aspect 10, wherein the base material is positioned on one or more surfaces of the composite mycelium material.

According to a twelfth aspect of the present disclosure, the composite mycelium material of any one of aspects 1 to 11, wherein the supporting material is selected from the group consisting of a mesh, a cheesecloth, a fabric, a knit fiber, a woven fiber, and a non-woven fiber.

According to a thirteenth aspect of the present disclosure, the composite mycelium material of any one of aspects 1 to 12, wherein the one or more masses of branching hyphae is disrupted by a mechanical action.

According to a fourteenth aspect of the present disclosure, the composite mycelium material of aspect 13, wherein the mechanical action includes blending the one or more masses of branching hyphae.

According to a fifteenth aspect of the present disclosure, the composite mycelium material of aspect 13, wherein the mechanical action includes applying a physical force to the one or more masses of branching hyphae such that at least some of the masses of branching hyphae are aligned in a parallel formation.

According to a sixteenth aspect of the present disclosure, the composite mycelium material of aspect 15, wherein the physical force is a pulling force.

According to a seventeenth aspect of the present disclosure, the composite mycelium material of aspect 15, wherein the mechanical action includes applying the physical force in one or more directions such that the at least some of the masses of branching hyphae are aligned in parallel in one or more directions, wherein the physical force is applied repeatedly.

According to an eighteenth aspect of the present disclosure, the composite mycelium material of any one of aspects 1 to 17, wherein the one or more masses of branching hyphae is disrupted by chemical treatment.

According to a nineteenth aspect of the present disclosure, the composite mycelium material of aspect 18, wherein the chemical treatment includes contacting the one or more masses of branching hyphae with a base or other chemical agent in an amount sufficient to cause a disruption.

According to a twentieth aspect of the present disclosure, the composite mycelium material of aspect 19, wherein the base includes alkaline peroxide.

According to a twenty-first aspect of the present disclosure, the composite mycelium material of any one of aspects 1 to 20, wherein the bonding agent includes one or more reactive groups.

According to a twenty-second aspect of the present disclosure, the composite mycelium material of aspect 21, wherein the one or more reactive groups react with active hydrogen containing groups.

According to a twenty-third aspect of the present disclosure, the composite mycelium material of aspect 22, wherein the active hydrogen containing groups comprise amine, hydroxyl, and carboxyl groups.

According to a twenty-fourth aspect of the present disclosure, the composite mycelium material of any one of aspects 1 to 23, wherein the bonding agent includes an adhesive, a resin, a crosslinking agent, and/or a matrix.

According to a twenty-fifth aspect of the present disclosure, the composite mycelium material of any one of aspects 1 to 23, wherein the bonding agent is selected from the group consisting of transglutaminase, polyamide-epichlorohydrin resin (PAE), citric acid, genipin, alginate, a natural adhesive, and a synthetic adhesive.

According to a twenty-sixth aspect of the present disclosure, the composite mycelium material of any one of aspects 1 to 23, wherein the bonding agent is PAE.

According to a twenty-seventh aspect of the present disclosure, the composite mycelium material of aspect 26, wherein the PAE includes cationic azetidinium groups that react with active hydrogen containing groups including amine, hydroxyl, and carboxyl groups, in the one or more branches of hyphae.

According to a twenty-eighth aspect of the present disclosure, the composite mycelium material of aspect 25, wherein the natural adhesive includes a natural latex-based adhesive.

According to a twenty-ninth aspect of the present disclosure, the composite mycelium material of aspect 28, wherein the natural latex-based adhesive is leather glue or weld.

According to a thirtieth aspect of the present disclosure, the composite mycelium material of any one of aspects 1 to 29, wherein the composite mycelium material includes one or more proteins that are from a species other than a fungal species from which the cultivated mycelium material is generated.

According to a thirty-first aspect of the present disclosure, the composite mycelium material of aspect 30, wherein the one or more proteins is from a plant source.

According to a thirty-second aspect of the present disclosure, the composite mycelium material of aspect 31, wherein the plant source is a pea plant.

According to a thirty-third aspect of the present disclosure, the composite mycelium material of aspect 31, wherein the plant source is a soybean plant.

According to a thirty-fourth aspect of the present disclosure, the composite mycelium material of any one of aspects 1 to 33, wherein the composite mycelium material further includes a dye.

According to a thirty-fifth aspect of the present disclosure, the composite mycelium material of aspect 34, wherein the dye is selected from the group including an acid dye, a direct dye, a synthetic dye, a natural dye, and a reactive dye.

According to a thirty-sixth aspect of the present disclosure, the composite mycelium material of aspect 34, wherein the composite mycelium material is colored with the dye and the color of the composite mycelium material is substantially uniform on one or more surfaces of the composite mycelium material.

According to a thirty-seventh aspect of the present disclosure, the composite mycelium material of aspect 34, wherein the dye is present throughout the interior of the composite mycelium material.

According to a thirty-eighth aspect of the present disclosure, the composite mycelium material of any one of aspects 1 to 37, wherein the composite mycelium material further includes a plasticizer.

According to a thirty-ninth aspect of the present disclosure, the composite mycelium material of aspect 38, wherein the plasticizer is selected from the group including oil, glycerin, fat liquor, water, glycol, triethyl citrate, water, acetylated monoglycerides, and epoxidized soybean oil.

According to a fortieth aspect of the present disclosure, the composite mycelium material of aspect 38, wherein the composite mycelium material is flexible.

According to a forty-first aspect of the present disclosure, the composite mycelium material of any one of aspects 1 to 40, wherein an external force is applied to the cultivated mycelium material.

According to a forty-second aspect of the present disclosure, the composite mycelium material of aspect 41, wherein the external force is applied via heating and/or pressing.

According to a forty-third aspect of the present disclosure, the composite mycelium material of any one of aspects 1 to 42, wherein the composite mycelium material further includes a tannin.

According to a forty-fourth aspect of the present disclosure, the composite mycelium material of any one of aspects 1 to 43, wherein the composite mycelium material further includes a finishing agent.

According to a forty-fifth aspect of the present disclosure, the composite mycelium material of aspect 44, wherein the finishing agent is selected from the group consisting of urethane, wax, nitrocellulose, and a plasticizer.

According to a forty-sixth aspect of the present disclosure, the composite mycelium material of any one of aspects 1 to 45, wherein the composite mycelium material includes a mechanical property.

According to a forty-seventh aspect of the present disclosure, the composite mycelium material of any one of aspects 1 to 46, wherein the mechanical property includes a wet tensile strength, an initial modulus, an elongation percentage at the break, a thickness, and/or a slit tear strength.

According to a forty-eighth aspect of the present disclosure, the composite mycelium material of any one of aspects 1-46, wherein the composite mycelium material has a wet tensile strength of 0.05 MPa to 10 MPa.

According to a forty-ninth aspect of the present disclosure, the composite mycelium material of any one of aspects 1 to 46, wherein the composite mycelium material has a wet tensile strength of 5 MPa to 20 MPa.

According to a fiftieth aspect of the present disclosure, the composite mycelium material of any one of aspects 1 to 46, wherein the composite mycelium material has a wet tensile strength of 7 MPa.

According to a fifty-first aspect of the present disclosure, the composite mycelium material of any one of aspects 1 to 46, wherein the composite mycelium material has an initial modulus of 1 MPa to 100 MPa.

According to a fifty-second aspect of the present disclosure, the composite mycelium material of any one of aspects 1 to 46, wherein the composite mycelium material has an elongation percentage at the break of 1% to 25%.

According to a fifty-third aspect of the present disclosure, the composite mycelium material of any one of aspects 1 to 46, wherein the composite mycelium material has a thickness of 0.5 mm to 3.5 mm.

According to a fifty-fourth aspect of the present disclosure, the composite mycelium material of any one of aspects 1 to 46, wherein the composite mycelium material has a thickness of 2 mm.

According to a fifty-fifth aspect of the present disclosure, the composite mycelium material of any one of aspects 1 to 46, wherein the composite mycelium material has a slit tear strength of 5 N to 100 N.

According to a fifty-sixth aspect of the present disclosure, the composite mycelium material of any one of aspects 1 to 46, wherein the composite mycelium material has a slit tear strength of 50 N.

According to a fifty-seventh aspect of the present disclosure, the composite mycelium material of any one of aspects 1 to 56, wherein the composite mycelium material is produced using traditional paper milling equipment.

According to a fifty-eighth aspect of the present disclosure, a method of producing a composite mycelium material, the method includes: generating a cultivated mycelium material including one or more masses of branching hyphae; disrupting the cultivated mycelium material including the one or more masses of branching hyphae; and adding a bonding agent to the cultivated mycelium material; thus producing the composite mycelium material.

According to a fifty-ninth aspect of the present disclosure, the method of aspect 58, wherein the generating includes generating cultivated mycelium material on a solid substrate.

According to a sixtieth aspect of the present disclosure, the method of aspect 58 or 59, wherein the cultivated mycelium material includes one or more masses of disrupted branching hyphae.

According to a sixty-first aspect of the present disclosure, the method of any one of aspects 58 to 60, wherein the one or more masses of disrupted branching hyphae has a length of 0.1 mm to 5 mm.

According to a sixty-second aspect of the present disclosure, the method of aspect 61, wherein the one or more masses of disrupted branching hyphae has a length of 2 mm.

According to a sixty-third aspect of the present disclosure, the method of any one of aspects 58 to 62, further including incorporating a supporting material into the composite mycelium material.

According to a sixty-fourth aspect of the present disclosure, the method of aspect 63, wherein the supporting material has a pore size of 1/16th of an inch.

According to a sixty-fifth aspect of the present disclosure, the method of aspect 63, wherein the supporting material includes a reinforcing material.

According to a sixty-sixth aspect of the present disclosure, the method of aspect 65, wherein the reinforcing material is entangled within the composite mycelium material.

According to a sixty-seventh aspect of the present disclosure, the method of aspect 63, wherein the supporting material includes a base material.

According to a sixty-eighth aspect of the present disclosure, the method of aspect 67, wherein the base material is positioned on one or more surfaces of the composite mycelium material.

According to a sixty-ninth aspect of the present disclosure, the method of any one of aspects 58 to 68, wherein the supporting material is selected from the group consisting of a mesh, a cheesecloth, a fabric, a knit fiber, a woven fiber, and a non-woven fiber.

According to a seventieth aspect of the present disclosure, the method of any one of aspects 58 to 69, wherein the disrupting includes disrupting the one or more masses of branching hyphae by a mechanical action.

According to a seventy-first aspect of the present disclosure, the method of aspect 70, wherein the mechanical action includes blending the one or more masses of branching hyphae.

According to a seventy-second aspect of the present disclosure, the method of aspect 70, wherein the mechanical action includes applying a physical force to the one or more masses of branching hyphae such that at least some of the masses of branching hyphae are aligned in a parallel formation.

According to a seventy-third aspect of the present disclosure, the method of aspect 72, wherein the physical force is a pulling force.

According to a seventy-fourth aspect of the present disclosure, the method of aspect 72, wherein the mechanical action includes applying the physical force in one or more directions such that the at least some of the masses of branching hyphae are aligned in parallel in one or more directions, wherein the physical force is applied repeatedly.

According to a seventy-fifth aspect of the present disclosure, the method of any one of aspects 58 to 74, wherein the one or more masses of branching hyphae is disrupted by chemical treatment.

According to a seventy-sixth aspect of the present disclosure, the method of aspect 75, wherein the chemical treatment includes contacting the one or more masses of branching hyphae with a base or other chemical agent in an amount sufficient to cause a disruption.

According to a seventy-seventh aspect of the present disclosure, the method of aspect 76, wherein the base includes alkaline peroxide.

According to a seventy-eighth aspect of the present disclosure, the method any one of aspects 58 to 77, wherein the bonding agent includes one or more reactive groups.

According to a seventy-ninth aspect of the present disclosure, the method of aspect 78, wherein the one or more reactive groups react with active hydrogen containing groups.

According to an eightieth aspect of the present disclosure, the method of aspect 79, wherein the active hydrogen containing groups comprise amine, hydroxyl, and carboxyl groups.

According to an eighty-first aspect of the present disclosure, the method of any one of aspects 58 to 80, wherein the bonding agent includes an adhesive, a resin, a crosslinking agent, and/or a matrix.

According to an eighty-second aspect of the present disclosure, the method of any one of aspects 58 to 80, wherein the bonding agent is selected from the group consisting of transglutaminase, polyamide-epichlorohydrin resin (PAE), citric acid, genipin, alginate, a natural adhesive, and a synthetic adhesive.

According to an eighty-third aspect of the present disclosure, the method of any one of aspects 58 to 80, wherein the bonding agent is PAE.

According to an eighty-fourth aspect of the present disclosure, the method of aspect 83, wherein the PAE includes cationic azetidinium groups that react with active hydrogen containing groups including amine, hydroxyl, and carboxyl groups, in the one or more branches of hyphae.

According to an eighty-fifth aspect of the present disclosure, the method of aspect 82, wherein the natural adhesive includes a natural latex-based adhesive.

According to an eighty-sixth aspect of the present disclosure, the method of aspect 85, wherein the natural latex-based adhesive is leather glue or weld.

According to an eighty-seventh aspect of the present disclosure, the method of any one of aspects 58 to 86, further including adding one or more proteins that are from a species other than a fungal species from which the cultivated mycelium material is generated.

According to an eighty-eighth aspect of the present disclosure, the method of aspect 87, wherein the one or more proteins is from a plant source.

According to an eighty-ninth aspect of the present disclosure, the method of aspect 88, wherein the plant source is a pea plant.

According to a ninetieth aspect of the present disclosure, the method of aspect 88, wherein the plant source is a soybean plant.

According to a ninety-first aspect of the present disclosure, the method of any one of aspects 58 to 90, further including adding a dye to the cultivated mycelium material or the composite mycelium material.

According to a ninety-second aspect of the present disclosure, the method of aspect 91, wherein the dye is selected from the group including an acid dye, a direct dye, a synthetic dye, a natural dye, and a reactive dye.

According to a ninety-third aspect of the present disclosure, the method of aspect 91, wherein the composite mycelium material is colored with the dye and the color of the composite mycelium material is substantially uniform on one or more surfaces of the composite mycelium material.

According to a ninety-fourth aspect of the present disclosure, the method of aspect 91, wherein the dye is present throughout the interior of the composite mycelium material.

According to a ninety-fifth aspect of the present disclosure, the method of any one of aspects 58 to 94, further including adding a plasticizer to the cultivated mycelium material or the composite mycelium material.

According to a ninety-sixth aspect of the present disclosure, the method of aspect 95, wherein the plasticizer is selected from the group including oil, glycerin, fat liquor, water, glycol, triethyl citrate, water, acetylated monoglycerides, and epoxidized soybean oil.

According to a ninety-seventh aspect of the present disclosure, the method of aspect 95, wherein the composite mycelium material is flexible.

According to a ninety-eighth aspect of the present disclosure, the method of any one of aspects 58 to 97, further including applying an external force to the cultivated mycelium material.

According to a ninety-ninth aspect of the present disclosure, the method of aspect 98, wherein the external force is applied via heating and/or pressing.

According to a one hundredth aspect of the present disclosure, the method of any one of aspects 58-99, further including adding a tannin to the cultivated mycelium material or the composite mycelium material.

According to a one hundred first aspect of the present disclosure, the method of any one of aspects 58 to 100, further including adding a finishing agent to the composite mycelium material.

According to a one hundred second aspect of the present disclosure, the method of aspect 101, wherein the finishing agent is selected from the group consisting of urethane, wax, nitrocellulose, and a plasticizer.

According to a one hundred third aspect of the present disclosure, the method of any one of aspects 58 to 102, further including determining a mechanical property of the composite mycelium material.

According to a one hundred fourth aspect of the present disclosure, the method of any one of aspects 58 to 103, wherein the mechanical property includes a wet tensile strength, an initial modulus, an elongation percentage at the break, a thickness, and/or a slit tear strength.

According to a one hundred fifth aspect of the present disclosure, the method of any one of aspects 58 to 103, wherein the composite mycelium material has a wet tensile strength of 0.05 MPa to 10 MPa.

According to a one hundred sixth aspect of the present disclosure, the method of any one of aspects 58 to 103, wherein the composite mycelium material has a wet tensile strength of 5 MPa to 20 MPa.

According to a one hundred seventh aspect of the present disclosure, the method of any one of aspects 58 to 103, wherein the composite mycelium material has a wet tensile strength of 7 MPa.

According to a one hundred eighth aspect of the present disclosure, the method of any one of aspects 58 to 103, wherein the composite mycelium material has an initial modulus of 1 MPa to 100 MPa.

According to a one hundred ninth aspect of the present disclosure, the method of any one of aspects 58 to 103, wherein the composite mycelium material has an elongation percentage at the break of 1% to 25%.

According to a one hundred tenth aspect of the present disclosure, the method of any one of aspects 58 to 103, wherein the composite mycelium material has a thickness of 0.5 mm to 3.5 mm.

According to a one hundred eleventh aspect of the present disclosure, the method of any one of aspects 58 to 103, wherein the composite mycelium material has a thickness of 2 mm.

According to a one hundred twelfth aspect of the present disclosure, the method of any one of aspects 58 to 103, wherein the composite mycelium material has a slit tear strength of 5 N to 100 N.

According to a one hundred thirteenth aspect of the present disclosure, the method of any one of aspects 58 to 103, wherein the composite mycelium material has a slit tear strength of 50 N.

According to a one hundred fourteenth aspect of the present disclosure, the method of any one of aspects 58 to 113, wherein the composite mycelium material is produced using traditional paper milling equipment.

According to a one hundred fifteenth aspect of the present disclosure, a method of producing a material including mycelium includes: introducing a fungal inoculum and a nutrient source to a vessel of a bioreactor, wherein the nutrient source is compatible with the fungal inoculum for consumption by the same; introducing a liquid to the vessel to provide a mixture; incubating the mixture in the bioreactor under aerobic conditions to grow a biomass of mycelium having a plurality of branches of hyphae having a length of at least about 0.1 mm; after the step of incubating the mixture, collecting at least a portion of the biomass of mycelium and adjusting a concentration of the biomass of mycelium to a predetermined concentration; and web-forming the collected biomass of mycelium to form a hyphal network.

According to a one hundred sixteenth aspect of the present disclosure, method of aspect 105, wherein the mixture includes a carbon source, a nitrogen source, a mineral element source, a pH modifier, or combinations thereof.

According to a one hundred seventeenth aspect of the present disclosure, the method of aspect 116, wherein the mixture includes a pH modifier selected to maintain the pH of the mixture at a pH of from about 4 to about 6 during the step of incubating the mixture.

According to a one hundred eighteenth aspect of the present disclosure, the method of any one of aspects 115 to 117, wherein a temperature of the mixture during the step of incubating the mixture is from about 25° C. to about 30° C.

According to a one hundred nineteenth aspect of the present disclosure, the method of any one of aspects 115 to 118, further including: agitating the mixture, aerating the mixture, or both during the step of incubating the mixture.

According to a one hundred twentieth aspect of the present disclosure, the method of any one of aspects 115 to 119, further including: combining a bonding agent with the biomass of mycelium one of prior to the web-forming step, during the web-forming step, or after the web-forming step.

According to a one hundred twenty-first aspect of the present disclosure, the method of aspect 120, wherein the bonding agent includes an adhesive, a resin, a crosslinking agent, a polymeric matrix material, or combinations thereof.

According to a one hundred twenty-second aspect of the present disclosure, the method of any one of aspects 115 to 121, further including: disrupting the plurality of branches of hyphae one of prior to the web-forming step, during the web-forming step, or after the web-forming step.

According to a one hundred twenty-third aspect of the present disclosure, the method of aspect 122, wherein the disrupting includes mechanical disrupting, chemical disrupting, or both.

According to a one hundred twenty-fourth aspect of the present disclosure, the method of any one of aspects 115 to 123, further comprising entangling the plurality of branches of hyphae in the hyphal network.

According to a one hundred twenty-fifth aspect of the present disclosure, the method of aspect 124, wherein the step of entangling the plurality of branches of hyphae includes hydroentangling using a liquid jet configured to spray liquid at a pressure of from about 700 psi to about 900 psi and/or wherein the step of entangling the plurality of branches of hyphae includes hydroentangling using a liquid jet configured to spray liquid at a flow rate of from about 100 mL/min. to 300 mL/min.

According to a one hundred twenty-sixth aspect of the present disclosure, the method of aspect 124 or 125, wherein the entangling the plurality of branches of hyphae includes needle punching, felting, or hydroentangling.

According to a one hundred twenty-seventh aspect of the present disclosure, the method of any one of aspects 115 to 126, wherein the hyphal network defines a first hyphal network, and further wherein the method includes covering a portion of the first hyphal network with a second hyphal network.

According to a one hundred twenty-eighth aspect of the present disclosure, the method of aspect 127, interconnecting a portion of the first hyphal network with the second hyphal network.

According to a one hundred twenty-ninth aspect of the present disclosure, the method of any one of aspects 115 to 128, wherein the step of web-forming the collected biomass of mycelium includes depositing the biomass of mycelium on a supporting material.

According to a one hundred thirtieth aspect of the present disclosure, the method of aspect 129, wherein the supporting material includes a woven fiber, a non-woven fiber, a mesh, a perforated plastic, woodchips, a cheesecloth, a fabric, a knot fiber, a scrim, a textile, or combinations thereof.

According to a one hundred thirty-first aspect of the present disclosure, the method of aspect 129, further comprising entangling at least a portion of the plurality of branches of hyphae with the supporting material.

According to a one hundred thirty-second aspect of the present disclosure, the method of any one of aspects 115 to 131, further including: combining a reinforcing material with the biomass of mycelium one of prior to the web-forming step, during the web-forming step, or after the web-forming step.

According to a one hundred thirty-third aspect of the present disclosure, the method of any one of aspects 115 to 132, wherein web-forming includes wet-laying, air-laying, or dry-laying.

According to a one hundred thirty-fourth aspect of the present disclosure, the method of any one of aspects 115 to 133, further comprising combining one of natural fibers, synthetic fibers, or combinations thereof with the biomass of mycelium one of prior to the web-forming step, during the web-forming step, or after the web-forming step.

According to a one hundred thirty-fifth aspect of the present disclosure, the method of aspect 134, wherein the fibers have a length of less than about 25 mm.

According to a one hundred thirty-sixth aspect of the present disclosure, the method of any one of aspects 115 to 135, wherein the mixture comprises a surfactant that is a polymeric macromolecule including monomer units selected from at least one of propylene oxide and ethylene oxide.

According to a one hundred thirty-seventh aspect of the present disclosure, the method of any one of aspects 115 to 136, further comprising agitating the mixture in the bioreactor according to an agitation profile, the agitation profile comprising: a first phase comprising agitating the mixture at a first agitation rate for a predetermined period of time; and a second phase comprising increasing the agitation rate from the first agitation rate to a second agitation rate, greater than the first agitation rate, and wherein an agitation rate during at least one of the first phase and second phase is based on a level of dissolved oxygen in the mixture.

According to a one hundred thirty-eighth aspect of the present disclosure, the method of any one of aspects 115 to 137, wherein the fungal inoculum comprises spores of a mutant of Neurospora crassa.

According to a one hundred thirty-ninth aspect of the present disclosure, the method of any one of aspects 115 to 138, wherein the introducing a fungal inoculum and a nutrient source to a vessel of a bioreactor comprises introducing a fungal inoculum containing fresh spores.

According to a one hundred fortieth aspect of the present disclosure, the method of aspect 139, wherein the introducing a fungal inoculum and a nutrient source to a vessel of a bioreactor comprises introducing an initial amount of at least one nutrient to the vessel that is consumed as the biomass of mycelium grows, the method further comprising: supplying an additional amount of the at least one nutrient to the vessel based on consumption of a predetermined portion of the initial amount of the at least one nutrient.

According to a one hundred forty-first aspect of the present disclosure, the method of aspect 140, wherein the predetermined portion of the initial amount of the at least one nutrient is at least about 50% of the predetermined portion of the initial amount of the at least one nutrient.

According to a one hundred forty-second aspect of the present disclosure, the method of aspect 140, wherein the at least one nutrient comprises glucose.

According to a one hundred forty-third aspect of the present disclosure, method of producing a material comprising mycelium comprises: introducing a fungal inoculum and a nutrient source to a vessel of a bioreactor, wherein the nutrient source is compatible with the fungal inoculum for consumption by the same; introducing a liquid to the vessel to provide a mixture, wherein the liquid comprises a surfactant that is a polymeric macromolecule including monomer units selected from at least one of propylene oxide and ethylene oxide; incubating the mixture in the bioreactor under aerobic conditions to grow a biomass of mycelium having a plurality of branches of hyphae having a length of at least about 0.1 mm; after the step of incubating the mixture, collecting at least a portion of the biomass of mycelium and adjusting a concentration of the biomass of mycelium to a predetermined concentration; and drying the biomass of mycelium.

According to a one hundred forty-fourth aspect of the present disclosure, the method of aspect 143, wherein the drying the biomass of mycelium comprises one of an air drying process, thermal drying process, vacuum drying process, press-drying process, and combinations thereof.

According to a one hundred forty-fifth aspect of the present disclosure, the method of aspect 143 or 144, further comprising: web-forming the collected biomass of mycelium to form a hyphal network one of prior to or subsequent to the drying the biomass of mycelium.

According to a one hundred forty-sixth aspect of the present disclosure, the method of aspect 145, wherein the web-forming comprises wet-laying, air-laying, or dry-laying.

According to a one hundred forty-seventh aspect of the present disclosure, the method of aspect 145, further comprising: entangling the plurality of branches of hyphae in the hyphal network subsequent to the web-forming.

According to a one hundred forty-eighth aspect of the present disclosure, the method of any one of aspect 143 to 147, wherein a temperature of the mixture during the step of incubating the mixture is from about 25° C. to about 40° C.

According to a one hundred forty-ninth aspect of the present disclosure, the method of any one of aspect 143 to 148, further comprising: agitating the mixture, aerating the mixture, or both during the step of incubating the mixture.

According to a one hundred fiftieth aspect of the present disclosure, the method of any one of aspect 143 to 149, further comprising agitating the mixture in the bioreactor according to an agitation profile, the agitation profile comprising: a first phase comprising agitating the mixture at a first agitation rate for a predetermined period of time; and a second phase comprising increasing the agitation rate from the first agitation rate to a second agitation rate, greater than the first agitation rate, and wherein an agitation rate during at least one of the first phase and second phase is based on a level of dissolved oxygen in the mixture.

According to a one hundred fifty-first aspect of the present disclosure, the method of any one of aspect 143 to 150, wherein the fungal inoculum comprises spores of a mutant of Neurospora crassa.

According to a one hundred fifty-second aspect of the present disclosure, the method of any one of aspect 143 to 151, wherein the introducing a fungal inoculum and a nutrient source to a vessel of a bioreactor comprises introducing a fungal inoculum containing fresh spores.

According to a one hundred fifty-third aspect of the present disclosure, the method of any one of aspect 143 to 152, wherein the introducing a fungal inoculum and a nutrient source to a vessel of a bioreactor comprises introducing an initial amount of at least one nutrient to the vessel that is consumed by the fungal inoculum, the method further comprising: supplying an additional amount of the at least one nutrient to the vessel based on consumption of a predetermined portion of the initial amount of the at least one nutrient.

According to a one hundred fifty-fourth aspect of the present disclosure, the method of aspect 153, wherein the predetermined portion of the initial amount of the at least one nutrient is at least about 50% of the predetermined portion of the initial amount of the at least one nutrient.

According to a one hundred fifty-fifth aspect of the present disclosure, the method of aspect 153, wherein the at least one nutrient comprises glucose.

According to a one hundred fifty-sixth aspect of the present disclosure, the method of any one of aspect 143 to 155, wherein the surfactant comprises at least one material selected from propylene oxide polymers, propylene oxide block co-polymers, propylene oxide/ethylene oxide block co-polymers, polyether polyols, polypropylene glycol, and combinations thereof.

According to a one hundred fifty-sixth aspect of the present disclosure, the method of any one of aspect 143 to 155, wherein the surfactant is present in the mixture in an amount (by weight) of from about 0.01% to about 1%.

It will be understood by one having ordinary skill in the art that construction of the described disclosure and other components is not limited to any specific material. Other exemplary embodiments of the disclosure disclosed herein may be formed from a wide variety of materials, unless described otherwise herein.

It is also important to note that the construction and arrangement of the elements of the disclosure as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. Accordingly, all such modifications are intended to be included within the scope of the present disclosure. Other substitutions, modifications, changes, and omissions may be made in the design, operating conditions, and arrangement of the desired and other exemplary embodiments without departing from the spirit of the present disclosure.

It will be understood that any described processes or steps within processes described herein may be combined with other disclosed processes or steps to form structures within the scope of the present disclosure. The exemplary structures and processes disclosed herein are for illustrative purposes and are not to be construed as limiting.

1. A method of producing a material comprising mycelium, the method comprising:
introducing a fungal inoculum and a nutrient source to a vessel of a bioreactor, wherein the nutrient source is compatible with the fungal inoculum for consumption by the same;
introducing a liquid to the vessel to provide a mixture;
incubating the mixture in the bioreactor under aerobic conditions to grow a biomass of mycelium having a plurality of branches of hyphae having a length of at least about 0.1 mm;
after the step of incubating the mixture, collecting at least a portion of the biomass of mycelium and adjusting a concentration of the biomass of mycelium to a predetermined concentration; and
web-forming the collected biomass of mycelium to form a hyphal network.

2. The method of claim 1, wherein the mixture comprises a carbon source, a nitrogen source, a mineral element source, a pH modifier, or combinations thereof.

3. The method of claim 2, wherein the mixture comprises a pH modifier selected to maintain the pH of the mixture at a pH of from about 4 to about 6 during the step of incubating the mixture.

4. The method of any one of claims 1 to 3, wherein a temperature of the mixture during the step of incubating the mixture is from about 25° C. to about 40° C.

5. The method of any one of claims 1 to 4, further comprising:
agitating the mixture, aerating the mixture, or both during the step of incubating the mixture.

6. The method of any one of claims 1 to 5, further comprising:
combining a bonding agent with the biomass of mycelium one of prior to the web-forming step, during the web-forming step, or after the web-forming step.

7. The method of claim 6, wherein the bonding agent comprises an adhesive, a resin, a cross-linking agent, a polymeric matrix material, or combinations thereof.

8. The method of any one of claims 1 to 7, further comprising:
disrupting the plurality of branches of hyphae one of prior to the web-forming step, during the web-forming step, or after the web-forming step.

9. The method of claim 8, wherein the disrupting comprises mechanical disrupting, chemical disrupting, or both.

10. The method of any one of claims 1 to 9, further comprising:
entangling the plurality of branches of hyphae in the hyphal network.

11. The method of any one of claims 1 to 10, wherein the hyphal network defines a first hyphal network, and further wherein the method includes covering a portion of the first hyphal network with a second hyphal network.

12. The method of claim 11, further comprising:
interconnecting a portion of the first hyphal network with the second hyphal network.

13. The method of any one of claims 1 to 12, wherein the step of web-forming the collected biomass of mycelium comprises depositing the biomass of mycelium on a supporting material.

14. The method of claim 13, wherein the supporting material comprises at least one material selected from a woven fiber, non-woven fiber, mesh, perforated plastic, woodchips, cheesecloth, fabric, knot fiber, scrim, textile, and combinations thereof.

15. The method of claim 13, further comprising:
entangling at least a portion of the plurality of branches of hyphae with the supporting material.

16. The method of any one of claims 1 to 15, further comprising:
combining a reinforcing material with the biomass of mycelium one of prior to the web-forming step, during the web-forming step, or after the web-forming step.

17. The method of any one of claims 1 to 16, wherein web-forming comprises wet-laying, air-laying, or dry-laying.

18. The method of any one of claims 1 to 17, further comprising:
combining one of natural fibers, synthetic fibers, or a combination thereof with the biomass of mycelium one of prior to the web-forming step, during the web-forming step, or after the web-forming step.

19. The method of claim 18, wherein the fibers have a length of less than 25 millimeters.

20. The method of any one of claims 1-19, wherein the mixture comprises a surfactant that is a polymeric macromolecule including monomer units selected from at least one of propylene oxide and ethylene oxide.

21. The method of any one of claims 1-20, further comprising agitating the mixture in the bioreactor according to an agitation profile, the agitation profile comprising:
a first phase comprising agitating the mixture at a first agitation rate for a predetermined period of time; and
a second phase comprising increasing the agitation rate from the first agitation rate to a second agitation rate, greater than the first agitation rate, and
wherein an agitation rate during at least one of the first phase and second phase is based on a level of dissolved oxygen in the mixture.

22. The method of any one of claims 1-21, wherein the fungal inoculum comprises spores of a mutant of Neurospora crassa.

23. The method of any one of claims 1-22, wherein the introducing a fungal inoculum and a nutrient source to a vessel of a bioreactor comprises introducing a fungal inoculum containing fresh spores.

24. The method of any one of claims 1-23, wherein the introducing a fungal inoculum and a nutrient source to a vessel of a bioreactor comprises introducing an initial amount of at least one nutrient to the vessel that is consumed as the biomass of mycelium grows, the method further comprising:
supplying an additional amount of the at least one nutrient to the vessel based on consumption of a predetermined portion of the initial amount of the at least one nutrient.

25. The method of claim 24, wherein the predetermined portion of the initial amount of the at least one nutrient is at least about 50% of the predetermined portion of the initial amount of the at least one nutrient.

26. The method of claim 24, wherein the at least one nutrient comprises glucose.

27. A method of producing a material comprising mycelium, the method comprising:
introducing a fungal inoculum and a nutrient source to a vessel of a bioreactor, wherein the nutrient source is compatible with the fungal inoculum for consumption by the same;
introducing a liquid to the vessel to provide a mixture, wherein the liquid comprises a surfactant that is a polymeric macromolecule including monomer units selected from at least one of propylene oxide and ethylene oxide;
incubating the mixture in the bioreactor under aerobic conditions to grow a biomass of mycelium having a plurality of branches of hyphae having a length of at least about 0.1 mm;
after the step of incubating the mixture, collecting at least a portion of the biomass of mycelium and adjusting a concentration of the biomass of mycelium to a predetermined concentration; and
drying the biomass of mycelium.

28. The method of claim 27, wherein the drying the biomass of mycelium comprises one of an air drying process, thermal drying process, vacuum drying process, press-drying process, and combinations thereof.

29. The method of claim 27 or claim 28, further comprising:
web-forming the collected biomass of mycelium to form a hyphal network one of prior to or subsequent to the drying the biomass of mycelium.

30. The method of claim 29, wherein the web-forming comprises wet-laying, air-laying, or dry-laying.

31. The method of claim 29, further comprising:
entangling the plurality of branches of hyphae in the hyphal network subsequent to the web-forming.

32. The method of any one of claims 27-31, wherein a temperature of the mixture during the step of incubating the mixture is from about 25° C. to about 40° C.

33. The method of any one of claims 27-32, further comprising:
agitating the mixture, aerating the mixture, or both during the step of incubating the mixture.

34. The method of any one of claims 27-33, further comprising agitating the mixture in the bioreactor according to an agitation profile, the agitation profile comprising:
a first phase comprising agitating the mixture at a first agitation rate for a predetermined period of time; and
a second phase comprising increasing the agitation rate from the first agitation rate to a second agitation rate, greater than the first agitation rate, and
wherein an agitation rate during at least one of the first phase and second phase is based on a level of dissolved oxygen in the mixture.

35. The method of any one of claims 27-34, wherein the fungal inoculum comprises spores of a mutant of Neurospora crassa.

36. The method of any one of claims 27-35, wherein the introducing a fungal inoculum and a nutrient source to a vessel of a bioreactor comprises introducing a fungal inoculum containing fresh spores.

37. The method of any one of claims 27-36, wherein the introducing a fungal inoculum and a nutrient source to a vessel of a bioreactor comprises introducing an initial amount of at least one nutrient to the vessel that is consumed by the fungal inoculum, the method further comprising:
supplying an additional amount of the at least one nutrient to the vessel based on consumption of a predetermined portion of the initial amount of the at least one nutrient.

38. The method of claim 37, wherein the predetermined portion of the initial amount of the at least one nutrient is at least about 50% of the predetermined portion of the initial amount of the at least one nutrient.

39. The method of claim 37, wherein the at least one nutrient comprises glucose.

40. The method of any one of claims 27-39, wherein the surfactant comprises at least one material selected from propylene oxide polymers, propylene oxide block co-polymers, propylene oxide/ethylene oxide block co-polymers, polyether polyols, polypropylene glycol, and combinations thereof.

41. The method of any one of claims 27-40, wherein the surfactant is present in the mixture in an amount (by weight) of from about 0.01% to about 1%.
